SEQUENCE LISTING (38 sequences)

SEQ ID NO: 1 - DNA, 187 bp, Artificial Sequence
  source: /note="Description of Artificial Sequence: Synthetic polynucleotide"
aaaaatgtgatcttgcttgtaaatacaattttgagaggttaataaattacaagtagtgctatttttgtatttaggttagctatttagctttacgttccaggatgcctagtggcagccccacaatatccaggaagccctctctgcggtttttcagattaggtagtcgaaaaacctaagaaatttacct

SEQ ID NO: 2 - DNA, 848 bp, Artificial Sequence
  source: /note="Description of Artificial Sequence: Synthetic polynucleotide"
acgcgctaccacacgccaggcaaacccacacgccgccttcccgccgcctccgctcttcttcccccaaccgcgtcgcaagcccacggcgagcgccgccgccgtcccaccgtcgccgatgacccgcggcgtcgctatggcgcacgcgaggctcctcctggcgcgctactacgcgatggcggcccccagttggcctaccgtatccaagaaccttccgctgctgggccatggacgttctcatcatccgatgtatgcaagtcaagacgagattaaaatgtcaagtagaagatggtgccatggttctcctgacaatcaagagctggcaaagaagatttgtgttccatgcaattctaaggatatacatgccatgccagaagattctgctaagaagatgctagaacaagtgggtggttgggaattagcaaccgaaggtgacattctgaaattgcacagagcatggaaggtgaagaattttgtaaaagggcttgaattccttcagcttgttgctgctgttgctgaggaagaaggccaccaccctgatcttcatcttgtcggttggaacaatgtgaaaattgatgtttggactcactctgtcagaggcttaactgacaatgatttcatccttgctgcgaagatcaataacctcaacttagaaggcctcttaagcaagaaagctacagtccaaaagtagttcgcgcagatgaagcgattgatttctgacgagatttatgtacttgcttcggttagaatgcgtgagttgaggtatctttaacttgataagcttttcttgaatttgtaccgggagtttctctgttctttctaaattttaccaatgctgttggaccgggtct

SEQ ID NO: 3 - DNA, 621 bp, Artificial Sequence
  source: /note="Description of Artificial Sequence: Synthetic polynucleotide"
atggtggaggagctcggcttcgacatcaatgccggcagagagcttggcataacagctttggcttctgctgcgttggatggaaggctggctactgcaaggtaccttcttgaccatggggctgatccaaataagaagtgcaatgcaggctctgacaagcaggatgatcataataaaaaggctcagttcaaactacgtggtgagaaagctattaaggacaagcatgatgagcaggataaaaaggctcagctcaaactacagggtgagaaagctgttaagagaaaggactaccatggcgcctcaattttctacactgaggcaattgagctggatcctactgatgcaacattgtattccaataggagcctttgccatcttcaaatgaccgaagctttgtttgatgctgattattgcataaaatcgcggcctgaatggttaaaaggttactacaggaaaggggctgctctcatgttgctgaaggagtatgaaaaggcatgtgacgcattcttggctggactgaagttggatcctttgaatgctgagatggagaaagtattccgggaggcggtcgaggcgatgaagaagcatcatgttactacaaaaagcttcaagccatcagattag

SEQ ID NO: 4 - DNA, 1393 bp, Artificial Sequence
  source: /note="Description of Artificial Sequence: Synthetic polynucleotide"
ggtgaggtgaggtgagttgagttggggattgattgattgattcggattgggaagaagaagaagcaggggagcatgggcagcaagtcgtacccgacggtgagcgatgagtacctggcggccgtgggcaaggcgaagcgcaagctccgcggcctcatcgccgagaagaactgcgccccactcatgctccgcctcgcgtggcactctgctggcaccttcgatgtgtcgtcgaggaccggcgggcccttcggcaccgtgaagaaccccggcgagcagtcccacgccgccaacgccggcctcgacatcgccgtcaggcttctcgaccccatcaaggaccaacttcccatcctctcctacgccgacttctaccagcttgctggcgttgtggccgtcgaggtcaccggcggacctgaggtccccttccatccgggcaggcaggacaagcctgagcctcctcctgaaggccgtcttcctgatgccacacaaggttctgaccacctaaggcaggtcttttctgcgcagatgggtttgagtgacaaggacatagttgctctttctggtggtcacaccctgggaagatgccacaaggagagatctggctttgagggagcctggacgtccatccctttgatctttgacaactcttacttcaccgagcttgtgagtggcgagaaggaaggccttcttcagctgccaagtgacaaagccctcatggctgacccagccttccgtccactggtggagaaatatgctgcggatgaggacgccttctttgcggattacgccgaggcccacctcaagctctctgaactgggatttgctgaggaataagaagcctttagagagcgggatatccgcaaaagattaatgccgatttgtattttgcgccttagagtcagtacgatcaagactgtcgtggcggttgtaataaaaattagtgtgctttgggccatctttttatgtgattccaattgtctttctcttcattcttgctttgatgctctttgtctggacctctagaccgccgtattgtactgtggagtttcaaagttaccaagctatttgctgtcaagataactatggattgaattccccttgatggatgaaccaactgttgttgtttgcccgttcttcagctttcgtttgtgcggccatcgatcgccatgcgttgcttaaacccatttctagctcccctaccctgctgcatccgccctcttctgcgcgatcgttggattgcgagtggttggctggttgcacgacttgtggagaccgaaacaaataatttttggtcaaattgatcggtggtactgtcggagcatctattttttctttagcttagatcgtataattgtaggattgggatttgtatattaatatatacaggtcgattaaaact

SEQ ID NO: 5 - protein, 190 aa, Artificial Sequence
  source: /note="Description of Artificial Sequence: Synthetic polypeptide"
MTRGVAMAHARLLLARYYAMAAPSWPTVSKNLPLLGHGRSHHPMYASQDEIKMSSRRWCHGSPDNQELAKKICVPCNSKDIHAMPEDSAKKMLEQVGGWELATEGDILKLHRAWKVKNFVKGLEFLQLVAAVAEEEGHHPDLHLVGWNNVKIDVWTHSVRGLTDNDFILAAKINNLNLEGLLSKKATVQK

SEQ ID NO: 6 - DNA, 1803 bp, Artificial Sequence
  source: /note="Description of Artificial Sequence: Synthetic polynucleotide"
ccaacactaatctaagctcaaaacaaacacaacttcctaacgtagtcacagagtcaaaaacattgtttcacttaaacacattcgcacataaaaagaaaccattaacatttaaccatactactagattattaattactagtacttgtgttgttgtgtcactcaaactaaactaaactcaatttccaatttccttaatcttcttgtgttgatgtgcatggagagaatcagagtgaaggaggaagaagcaatgacatgtggaggtggtggttcatcatcttcaagctttatttctccacaggcacagcccatggaagggttgcacgaggtgggtccccctccgtttctgagcaagatctttgacatggtggaagatccttccacggattccattgtttcatggagcatggctcgcaacagctttgtcgtgtgggactctcacaagttttctgctcacattctgcctcgttacttcaagcacgccaatttctccagcttcattcgccagctcaacacctatgggtttaggaaagttgatcctgataaatgggaatttgcaaacgaaggttttctggcaggacaaaggcacttattgaagaccataaagagaagaagaaatgtgtcacacagcaatcaacaaaaaggaggaagtggagcttgtgttgaagtggggaagtttggactagaaggtgagttggagaggttgaaaagggatagaaacattttgatggcagaaattgtgagattgaggcaccaacaactgaactcaagggaccaattaagtgccatggaggcaagaatgcaagccacagagaagaaacagcaacaaatgatgagtttcttagcaaaagcactaagcaatccatctttcatgcagcaattagtccacaaaaccccccaaagcagagaagtgttgttgggtgttgaaatcaataggaaaaggagactacctgcttgtccaagtgtggagaacttgcaacaagataatcaagatttggcaactatggagactgatatggacacttttttcgcaccggcttatgataatgaattcggtaatgaaatcgatgagcctgcatcgattttggttgaggattcaatcttggaggactttctcaacaaagacttgattactgggaatccggaggatgaagttataattggtgattgcacccaagtcgatgtgccgatggaggatttggtagcaaaccctgatgattggagtgagcaattgcaggaccttgtggatcatatggactacctaggatccaaaccatagagggatgagaattatactattattgtgtcctcagcttggtctctttaatttcattcacattgcatcatgatggtacaatttggtacttttgttactacttttggttttggtttcaattttatgtgatcttaatgataagtgtgaattgtttttatgcagcagggaaggtggtagatacagaagaagcaaacaaatactgctcttgtttggatcctttatgaagttttatgaatattacttgtgctaatgctacagatcattttattaattatttggatgacctatttgtttgcattttatgttatgcaggagagacaagtgggggcacttcatggttgtgatccaaataattgctgagaaattgtaatggtatcattctgcttcataggatatcattggctggatttcattgttcaggagaggatataaaaactatcatctagttcttctagtacgaatagcctttcagaggaaccacatttgttcccattctttttccttcaaaaaattggatattgttggtgcta

SEQ ID NO: 7 - DNA, 3478 bp, Artificial Sequence
  source: /note="Description of Artificial Sequence: Synthetic polynucleotide"
aggtgcattccttcttataattttttcaccttcattattattaattaaaagtcccaataaacaaaggaaaaagttctatcacttttttacaattggaaaaaagagattcttttaaggaatgtgtggtgacaaataagcatcctattttcttctgttactaaagcctctaaaaaaataaaaaataaaaatatatatacttaaactcacaagtttagggcaattttgatgcatctttattttgtttatcaaactctatatatagtcattcataaaaatgatagtacgacacataaatcactccatattttattatattgtaattatgattcgagaaaggatttaatttctcaaaatataatataaatagactatcgtaatataaatattagtactatctaatgaagtacgatttgatgaagtgtaaagttaacttacatatgacctagagaaaccacttaagtagcaatgaataatccaagatatatatatacttaaactcacaagttacatttttgacgtatctttattttgtttattaaactctatatatagtcattcataaaaatgataatatgacgcataaatcattccatattttattaaattataattaagatttgaaaaaagaatttacttcttcaaaatataatctaaatagtctatcgtaatataaatattaatgttatctagtgaagtatgatttaatgaagtgtaaagttaacttacatatgacctagagaaatcacttaagtagcgatgaataatccaagaatatgtttggtcctttattctttcttgtcatggctcatgtatccatgcactttattataacaattcgagaagtgttataattatggtgattctcttatttaaaattttttgaactattaataaagtaaatgaataataataataataatatgaattaggaaaatatttatgtatataatttttatgtcaaaattacttgattctccactttacagctcaacaattaacatatatggtttccccttaaagaaaaacttcaaaaagattcctatgatggtaaagaaacgtttggccataaaaattaaatatttttcaattttcaaatcgaaatttttttgatcactatggatctgatataaacagtctccctattacgaaaaagtaagagtaaggtctgcatacatcttatcctctttaaacctcacagtttgaagatgcgactttgtttgattatactttttccaaaggagagaattaagagattatatttggaattacgcagacaaaatttgaaagacatcttataagtttgaaatccaattacaagtggaatttaaaattttcacgacttgtcaaccattgattctcaaataaagtgaaaaattattccaaaaacaaataaatattttttttatgaccaaatatgtcctcaagaacatatgaaagctctctagtcatgagtataaataacaagggctagctagctcttgtctactcataaaatatcatccatccatctcatgtaataaacaaaaattgagcttattaattataattgagaagaaaaaaaatcatgcctagagatcctttaatagtttctggagttgttggagatgttgttgatccattcacaagatgtgtagactttggtgtggtttacaacaatagggtggtctataatggatgttccttgaggccttcacaagttgtcaatcaacctagggttgacattgatggagacgatcttcgtactttttacactctggtataaactcatcgttttatttcatatgatatacatatatatatatatatatatatatatatatatatatattttctttctatttatacattttaatatctctaaattattaaccttttgtcaattgattatgagtagaagatcaaaaggacaatatgtgcaaaggcttctaattatgtgaatttgtgttagttttaattttgattcaccatctaagtacttgttttgtggtttttatttgaatttgagaactcataacatactatttatgataataaaaaatgttagtaacatgtatgtttaatattgcaagcttgaaaatatacaatatttttaaattactaataatgtcatgtaatacatttggatatacaatatggaaaattatttttcctaattttcaaaatatttgaaatgtttcttttctttttggaagattatggtggatcctgatgctccaaaccctagcaacccaaacctgagggaatatttgcactggtaagtcatctagcttatattatatatatatatatatatatatatatatattatataaatagataaaaatattcattttgttatatacttcttatttctcttaaatcaatcgtcgatagcgaagacaaaaatgtatgtgagattatataagaacctaaggaaagtattatttcataaaatgataactttctgatacacaaattaatcaatatttcaaataaataccaaatatcgaataacaacgtaaaaaaataataactattatcgattgcttaatccccttacaattaatgtacctaaacctctttttttttttaaaaaaaaaataataataataatgtttaacacattatttttttaataggttggtcacagatatcccagcagccacaggagcaacctttggtaagtttttcttacattattacctaatggctcgtaattacgcagtgacgaagcaagaaatttaaatatactttatatttacgatacattgtatccgtatcactacatttttaatataagacggttagtaatatacaaaatacaacttgtatcatcatcaccttagtagtacattattagtactataggcccaattatgactactaataaaataagacttaaaaagaaacataaaatcaaaatgaagtatatactatgtatataaatgtttttgaaacaaggaaaatacgcgtattgaatgtctttgttactaaactcaaactctcgttatacaggcaatgaagtcgtgggctacgagagcccacgaccctcaatgggaatccatcgttatattttcgtgttgtatcgacaattgggctgcgatgccatcgatgcaccggacataatcgattctagacaaaatttcaacacaagagactttgctaggtttcacaatctaggtttgcctgttgctgctgtttacttcaattgcaatagggaaggtggtaccggtggtcgtcgcctataaatcacccccctctcctcggggtgcgatccgttctcgaactctgtgtcaatgtcagatgttttgtgtaacggattttttgtttgatagtcactcagctaaattgcttattaactcagaaagtcatttttctttttatcgaagaaaattgaaattatgagataatagctattatagttgagtgataatctgaaaaaaaaaatcaaccataatatatattatatattatatatatacccaaaaaataaaagtct

SEQ ID NO: 8 - protein, 175 aa, Artificial Sequence
  source: /note="Description of Artificial Sequence: Synthetic polypeptide"
MPRDPLIVSGVVGDVVDPFTRCVDFGVVYNNRVVYNGCSLRPSQVVNQPRVDIDGDDLRTFYTLIMVDPDAPNPSNPNLREYLHWLVTDIPAATGATFGNEVVGYESPRPSMGIHRYIFVLYRQLGCDAIDAPDIIDSRQNFNTRDFARFHNLGLPVAAVYFNCNREGGTGGRRL

SEQ ID NO: 9 - DNA, 4641 bp, Artificial Sequence
  source: /note="Description of Artificial Sequence: Synthetic polynucleotide"
gtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcattaattaataatacgactcactatagggagaagatctttgtcgatcctaccatccactcgacacacccgccagcggccgccctaacgttactggccgaagccgcttggaataaggccggtgtgcgtttgtctatatgttattttccaccatattgccgtcttttggcaatgtgagggcccggaaacctggccctgtcttcttgacgagcattcctaggggtctttcccctctcgccaaaggaatgcaaggtctgttgaatgtcgtgaaggaagcagttcctctggaagcttcttgaagacaaacaacgtctgtagcgaccctttgcaggcagcggaaccccccacctggcgacaggtgcctctgcggccaaaagccacgtgtataagatacacctgcaaaggcggcacaaccccagtgccacgttgtgagttggatagttgtggaaagagtcaaatggctcccctcaagcgtattcaacaaggggctgaaggatgcccagaaggtaccccattgtatgggatctgatctggggcctcggtgcacatgctttacatgtgtttagtcgaggttaaaaaacgtctaggccccccgaaccacggggacgtggttttcctttgaaaaacacgatgataataccatggtgtctaagggcgaagagctgattaaggagaacatgcacatgaagctgtacatggagggcaccgtgaacaaccaccacttcaagtgcacatccgagggcgaaggcaagccctacgagggcacccagaccatgagaatcaaggtggtcgagggcggccctctccccttcgccttcgacatcctggctaccagcttcatgtacggcagcaaaaccttcatcaaccacacccagggcatccccgacttctttaagcagtccttccctgagggcttcacatgggagagagtcaccacatacgaggacgggggcgtgctgaccgctacccaggacaccagcctccaggacggctgcctcatctacaacgtcaagatcagaggggtgaacttcccatccaacggccctgtgatgcagaagaaaacactcggctgggaggcctccaccgagatgctgtaccccgctgacggcggcctggaaggcagaagcgacatggccctgaagctcgtgggcgggggccacctgatctgcaacttgaaaaccacatacagatccaagaaacccgctaagaacctcaagatgcccggcgtctactatgtggacaggagactggaaagaatcaaggaggccgacaaagagacctacgtcgagcagcacgaggtggctgtggccagatactgcgacctccctagcaaactggggcacaaacttaattgactcgaggtttctagaagttgtctcctcctgcactgactgactgatacaatcgatttctggatccgcaggcctctgctagcttgactgactgagatacagcgtaccttcagctcacagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggaggtgtgggaggttttttaaagcaagtaaaacctctacaaatgtggtattggcccatctctatcggtatcgtagcataaccccttggggcctctaaacgggtcttgaggggttttttgtgcccctcgggccggattgctatctaccggcattggcgcagaaaaaaatgcctgatgcgacgctgcgcgtcttatactcccacatatgccagattcagcaacggatacggcttccccaacttgcccacttccatacgtgtcctccttaccagaaatttatccttaaggtcgtcagctatcctgcaggcgatctctcgatttcgatcaagacattcctttaatggtcttttctggacaccactaggggtcagaagtagttcatcaaactttcttccctccctaatctcattggttacctgatgcggtattttctccttacgcatctgtgcggtatttcacaccgcatacgtcaaagcaaccatagtacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgatttgggtgatggttcacgtagtgggccatcgccctgatagacggtttttcgccctttgacgttggagtccacgttctttaatagtggactcttgttccaaactggaacaacactcaaccctatctcgggctattcttttgatttataagggattttgccgatttcggcctattggttaaaaaatgagctgatttaacaaaaatttaacgcgaattttaacaaaatattaacgtttacaattttatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtggaagccgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagacccc

SEQ ID NO: 10 - DNA, 110 bp, Artificial Sequence
  source: /note="Description of Artificial Sequence: Synthetic polynucleotide"
ggcagcagagagacctgaaaaagcaatttttctggaatttcagctgtttccaaactcaataagtatcttctagcaagagggaataggtgggaaaaaaaaaaagagatttc

SEQ ID NO: 11 - DNA, 497 bp, Artificial Sequence
  source: /note="Description of Artificial Sequence: Synthetic polynucleotide"
cgttgacttgcgcaagtcaacgttaacaccgttttcggggggtgacgatcgtcaccccaatgggtaagggttccatcgccgggaaacagctggagcttagtaatgaataaatgaactggactgactaatgtagtcagtccctcgatcgatgcatgcataccgtaaaggagaagctggtggaacgtgggatctctgtgcaaagctacaatggagatctattgtatgaaccgtgggaatatcggccggccacgcgacagtgcgctcgggtggtccatccactcgacacacccgccaccggcagccgctacccgcgcgcggccggtatattctggccttcccacaagggaaggccaggatcgtcgatgccgtggccaaggggtcattgagatagcctttcatgatttccgtctgcgcgcgcagacgggcaaggtagaagaccttggctgtaccattttttaacggaggagttactttctaagtatcatttagatttgaac

SEQ ID NO: 12 - DNA, 273 bp, Artificial Sequence
  source: /note="Description of Artificial Sequence: Synthetic polynucleotide"
agtacccttcaccaaagcaaaaatgtgatcttgcttgtaaatacaattttgagaggttaataaattacaagtagtgctatttttgtatttaggttagctatttagctttacgttccaggatgcctagtggcagccccacaatatccaggaaggtgccatgccctctctgcggtttttcagattaggtagtcgaaaaacctaagaaatttacctgctagggcatggcactcttgaaaaagtcatgccgtttcatatgatccatttcaagatacc

SEQ ID NO: 13 - DNA, 269 bp, Artificial Sequence
  source: /note="Description of Artificial Sequence: Synthetic polynucleotide"
agtacccttcaccaaagcaaaaatgtgatcttgcttgtaaatacaattttgagaggttaataaattacaagtagtgctatttttgtatttaggttagctatttagctttacgttccaggatgcctagtggcagccccacaatatccaggaaactcttgaaaaagtcatgccgtttcatatgatctggccctctctgatcatatgaaacggtttttcagattaggtagtcgaaaaacctaagaaatttacctgctacatttcaagatacc

SEQ ID NO: 14 - DNA, 273 bp, Artificial Sequence
  source: /note="Description of Artificial Sequence: Synthetic polynucleotide"
agtacccttcaccaaagcaaaaatgtgatcttgcttgtaaatacaattttgagaggttaataaattacaagtagtgctatttttgtatttaggttagctatttagctttacgttccaggatgcctagtggcagccccacaatatccaggaagggaaaactgaccctctctgcggtttttcagattaggtagtcgaaaaacctaagaaatttacctgctatcagggtcagttttccgtaggtggcgtcaccctccccttccatttcaagatacc

SEQ ID NO: 15 - DNA, 276 bp, Artificial Sequence
  source: /note="Description of Artificial Sequence: Synthetic polynucleotide"
agtacccttcaccaaagcaaaaatgtgatcttgcttgtaaatacaattttgagaggttaataaattacaagtagtgctatttttgtatttaggttagctatttagctttacgttccaggatgcctagtggcagccccacaatatccaggaaggaccaaacccctctctgcggtttttcagattaggtagtcgaaaaacctaagaaatttacctgctatcaggggtttggtctgcttggatgcggataccatcataaaggaaccatttcaagatacc

SEQ ID NO: 16 - DNA, 314 bp, Artificial Sequence
  source: /note="Description of Artificial Sequence: Synthetic polynucleotide"
gattcctaaattactacttgtaatttgggaatcgcaacacaacatggttacccatagattgaggaaatttccaataaactcagtattaaggcttgttgtgttggacaaggtgccctatttagggtgaggagccttactggcagccccagtgaatcctcaagaagatggaaacattggataggaacagctatattgggtagttgtagcagttgtattcaaatgaatgcagcgttccgaaatatcatacctgccgatcagaatgtttccatcttctttaaaatcaataccttttaactcgattcaagaaacaaata

SEQ ID NO: 17 - DNA, 309 bp, Artificial Sequence
  source: /note="Description of Artificial Sequence: Synthetic polynucleotide"
gattcctaaattactacttgtaatttgggaatcgcaacacaacatggttacccatagattgaggaaatttccaataaactcagtattaaggcttgttgtgttggacaaggtgccctatttagggtgaggagccttactggcagccccagtgaatcctcgcctgagactcattggataggaacagctatattgggtagttgtagcagttgtattcaaatgaatgcagcgttccgaaatatcatacctgccgatcatgagtctcaggctttacctctggtttgacttcaggtttggcttaagaaacaaata

SEQ ID NO: 18 - DNA, 268 bp, Artificial Sequence
  source: /note="Description of Artificial Sequence: Synthetic polynucleotide"
agtacccttcaccaaagcaaaaatgtgatcttgcttgtaaatacaattttgagaggttaataaattacaagtagtgctatttttgtatttaggttagctatttagctttacgttccaggatgcctagtggcagccccacaatatccaggaagccgctgccctctctgcggtttttcagattaggtagtcgaaaaacctaagaaatttacctgctattgggcagcggctgtccgaagccatgtctgatattgatgcatttcaagatacc

SEQ ID NO: 19 - DNA, 280 bp, Artificial Sequence
  source: /note="Description of Artificial Sequence: Synthetic polynucleotide"
agtacccttcaccaaagcaaaaatgtgatcttgcttgtaaatacaattttgagaggttaataaattacaagtagtgctatttttgtatttaggttagctatttagctttacgttccaggatgcctagtggcagccccacaatatccaggaagaagacccagtccctctctgcggtttttcagattaggtagtcgaaaaacctaagaaatttacctgctatagggactgggtcttcgaatctaaagtagtaccaaaaatccagcctccatttcaagatacc

SEQ ID NO: 20 - DNA, 313 bp, Artificial Sequence
  source: /note="Description of Artificial Sequence: Synthetic polynucleotide"
gattcctaaattactacttgtaatttgggaatcgcaacacaacatggttacccatagattgaggaaatttccaataaactcagtattaaggcttgttgtgttggacaaggtgccctatttagggtgaggagccttactggcagccccagtgaatcctcgtgctatccccattggataggaacagctatattgggtagttgtagcagttgtattcaaatgaatgcagcgttccgaaatatcatacctgccgatcacatggggatagcactactaaaattaattttacacattagggctcttcaagaaacaaata

SEQ ID NO: 21 - DNA, 301 bp, Artificial Sequence
  source: /note="Description of Artificial Sequence: Synthetic polynucleotide"
gattcctaaattactacttgtaatttgggaatcgcaacacaacatggttacccatagattgaggaaatttccaataaactcagtattaaggcttgttgtgttggacaaggtgccctatttagggtgaggagccttactggcagccccagtgaatcctccgccgccattggataggaacagctatattgggtagttgtagcagttgtattcaaatgaatgcagcgttccgaaatatcatacctgccgatccgatggcggcggccgtgggctcgttgatgatccgcagcacaagaaacaaata

SEQ ID NO: 22 - DNA, 273 bp, Artificial Sequence
  source: /note="Description of Artificial Sequence: Synthetic polynucleotide"
agtacccttcaccaaagcaaaaatgtgatcttgcttgtaaatacaattttgagaggttaataaattacaagtagtgctatttttgtatttaggttagctatttagctttacgttccaggatgcctagtggcagccccacaatatccaggaagaacaaggagccctctctgcggtttttcagattaggtagtcgaaaaacctaagaaatttacctgctaccgggctccttgttcttgtccgggtggtagcgcagcgcctgcatttcaagatacc

SEQ ID NO: 23 - DNA, 304 bp, Artificial Sequence
  source: /note="Description of Artificial Sequence: Synthetic polynucleotide"
gattcctaaattactacttgtaatttgggaatcgcaacacaacatggttacccatagattgaggaaatttccaataaactcagtattaaggcttgttgtgttggacaaggtgccctatttagggtgaggagccttactggcagccccagtgaatcctccagtgcaccattggataggaacagctatattgggtagttgtagcagttgtattcaaatgaatgcagcgttccgaaatatcatacctgccgatcagatggtgcactggacagaaggatatcctggcaggaacctcaagaaacaaata

SEQ ID NO: 24 - DNA, 270 bp, Artificial Sequence
  source: /note="Description of Artificial Sequence: Synthetic polynucleotide"
agtacccttcaccaaagcaaaaatgtgatcttgcttgtaaatacaattttgagaggttaataaattacaagtagtgctatttttgtatttaggttagctatttagctttacgttccaggatgcctagtggcagccccacaatatccaggaagaatgctgccctctctgcggtttttcagattaggtagtcgaaaaacctaagaaatttacctgctagtgggcagcattcttttgccaacaagttcttagaggaggccatttcaagatacc

SEQ ID NO: 25 - DNA, 421 bp, Artificial Sequence
  source: /note="Description of Artificial Sequence: Synthetic polynucleotide"
gccagccccctgatgggggcgacactccaccatgaatcactcccctgtgaggaactactgtcttcacgcagaaagcgtctagggataggttcccgatcacaacttgtcaggtcgaccccatggcgttagtatgagtgtcgtgcagcctccaggaccccccctcccgggagagccatagtggtctgcggaaccggtgagtacaccggaattgccaggacgaccgggtcctttcttggataaacccgctcaatgcctggagatttgggcgtgcccccgcaagactgctagccgagtagtgttgggtcgcgaaaggcctggtcgacctgactgtggtactgcctgatagggtgcttgcgagtgccccgggaggtctcgtagaccgtgcaccatgagcacgaatcctaaacctcaaagaaaaacc

SEQ ID NO: 26 - DNA, 794 bp, Artificial Sequence
  source: /note="Description of Artificial Sequence: Synthetic polynucleotide"
ttaaaacagctctggggttgtacccaccccagaggcccacgtggcggctagtactccggtattgcggtacccttgtacgcctgttttatactcccttcccgtaacttagacgcacaccctcgaggtagctatacagcgggtacatgagcgcaaaccaagttcaatagaagggggtacaaaccagtaccaccacgaacaagcacgcgctcatttacccgttctgtttccccggtgatgtcgtatagactgcttgcgtggttgaaagcgacggatccgttatccgcttatgtacttcgagaagcccagtaccacctcggaatcttcgatgcgttgcgctcagcactcaaccccagagtgtagcttaggctgatgagtctggacatccctcaccggtgacggtggtccaggctgcgttggcggcctacctatggctaacgccatgggacgctagttgtgaacaaggtgtgaagagcctattgagctacataagaatcctccggcccctgaatgcggctaatcccaacctcggagcaggtggtcacaaaccagtgattggcctgtcgtaacgcgcaagtccgtggcggaaccgactactttgggtgtccgtgtttccttttattttattgtggctgcttatggtgacaatcacagattgttatcataaagcgaattggattggccatccggtgaaagtgagactcattatctatctgtttgctggatccgctccattgagtgtgtttactctaagtacaatttcaacagttatttcaatcagacaattgtatcatatg

SEQ ID NO: 27 - DNA, 776 bp, Artificial Sequence
  source: /note="Description of Artificial Sequence: Synthetic polynucleotide"
cacccactcacagggcccactgggcgctagcactctgattttacgaaatccttgtgcgcctgttttatatcccttccggtggcgcaccagcggtaaccatccgagcggctaattcgaaacgtagaagcaatgcgcaccactccgctcgtatggttagatcaatagtaggcgtaacgcgccagttacgtcatgatcaagcatatctgttcccccggactgagtatcaatagactgcttacgcggttgaaggagaaaacgttcgttatccggctaactacttcgagaagcccagtaacaccatggaagctgcagggtgtttcgctcagcacttcccccgtgtagatcaggtcgatgagccactgcaatccccacaggtgactgtggcagtggctgcgttggcggcctgcctatggggagacccataggacgctctaatgtggacatggtgcgaagagcctattgagctagttagtagtcctccggcccctgaatgcggctaatcctaactgcggagcacatgccttcaacccagagggtagtgtgtcgtaacgggcaactctgcagcggaaccgactactttgggtgtccgtgtttctttttatttttatattggctgcttatggtgacaattacagaattgttaccatatagctattggattggccatccggtgtgtaatagagctgttatatacctatttgttggctttgtaccactaactttaaaatctataactaccctcaactttgtattaactctcaatacagctgatcatg

SEQ ID NO: 28 - DNA, 796 bp, Artificial Sequence
  source: /note="Description of Artificial Sequence: Synthetic polynucleotide"
gacatagactgagctgatagtatgttttgctaaagtgaggttaaaacagctctggggttgtacccaccccagaggcccacgtggcggctagtactccggtattgcggtacccttgtacgcctgttttatactcccttcccgtaacttagacgcacaaaaccaagttcaatagaagggggtacaaaccagtaccaccacgaacaagcacttctgtttccccggtgatgtcgtatagactgcttgcgtggttgaaagcgacggatccgttatccgcttatgtacttcgagaagcccagtaccacctcggaatcttcgatgcgttgcgctcagcactcaaccccagagtgtagcttaggctgatgagtctggacatccctcaccggtgacggtggtccaggctgcgttggcggcctacctatggctaacgccatgggacgctagttgtgaacaaggtgtgaagagcctattgagctacataagaatcctccggcccctgaatgcggctaatcccaacctcggagcaggtggtcacaaaccagtgattggcctgtcgtaacgcgcaagtccgtggcggaaccgactactttgggtgtccgtgtttccttttattttattgtggctgcttatggtgacaatcacagattgttatcataaagcgaattggattggccatcctcactttagcccggtgaaagtgagactcattatctatctgtttgctggatccgctccattgagtgtgtttactctaagtacaatttcaacagttatttcaatcagacaattgtatcatatg

SEQ ID NO: 29 - DNA, 268 bp, Artificial Sequence
  source: /note="Description of Artificial Sequence: Synthetic polynucleotide"
agtacccttcaccaaagcaaaaatgtgatcttgcttgtaaatacaattttgagaggttaataaattacaagtagtgctatttttgtatttaggttagctatttagctttacgttccaggatgcctagtggcagccccacaatatccaggaagccctctctgcggtttttcagattaggtagtcgaaaaacctaagaaatttacctgctacatttgtttcgcttcgcctattaccatccctgacccgtcgtcagcgaacccaagatacc

SEQ ID NO: 30 - DNA, 223 bp, Artificial Sequence
  source: /note="Description of Artificial Sequence: Synthetic polynucleotide"
agtacccttcaccaaagcaaaaatgtgatcttgcttgtaaatacaattttgagaggttaataaattacaagtagtgctatttttgtatttaggttagctatttagctttacgttccaggatgcctagtggcagccccacaatatccaggaagccctctctgcggtttttcagattaggtagtcgaaaaacctaagaaatttacctgctacatttcaagatacc

SEQ ID NO: 31 - DNA, 223 bp, Artificial Sequence; identical to SEQ ID NO: 30
  source: /note="Description of Artificial Sequence: Synthetic polynucleotide"

SEQ ID NO: 32 - DNA, 255 bp, Artificial Sequence
  source: /note="Description of Artificial Sequence: Synthetic polynucleotide"
gattcctaaattactacttgtaatttgggaatcgcaacacaacatggttacccatagattgaggaaatttccaataaactcagtattaaggcttgttgtgttggacaaggtgccctatttagggtgaggagccttactggcagccccagtgaatcctccattggataggaacagctatattgggtagttgtagcagttgtattcaaatgaatgcagcgttccgaaatatcatacctgccgatcaagaaacaaata

SEQ ID NO: 33 - DNA, 374 bp, Artificial Sequence
  source: /note="Description of Artificial Sequence: Synthetic polynucleotide"
gccagccccctgatgggggcgacactccaccatgaatcactcccctgtgaggaactactgtcttcacgcagaaagcgtctagccatggcgttagtatgagtgtcgtgcagcctccaggaccccccctcccgggagagccatagtggtctgcggaaccggtgagtacaccggaattgccaggacgaccgggtcctttcttggataaacccgctcaatgcctggagatttgggcgtgcccccgcaagactgctagccgagtagtgttgggtcgcgaaaggccttgtggtactgcctgatagggtgcttgcgagtgccccgggaggtctcgtagaccgtgcaccatgagcacgaatcctaaacctcaaagaaaaacc

SEQ ID NO: 34 - DNA, 743 bp, Artificial Sequence
  source: /note="Description of Artificial Sequence: Synthetic polynucleotide"
ttaaaacagctctggggttgtacccaccccagaggcccacgtggcggctagtactccggtattgcggtacccttgtacgcctgttttatactcccttcccgtaacttagacgcacaaaccaagttcaatagaagggggtacaaaccagtaccaccacgaacaagcacttctgtttccccggtgatgtcgtatagactgcttgcgtggttgaaagcgacggatccgttatccgcttatgtacttcgagaagcccagtaccacctcggaatcttcgatgcgttgcgctcagcactcaaccccagagtgtagcttaggctgatgagtctggacatccctcaccggtgacggtggtccaggctgcgttggcggcctacctatggctaacgccatgggacgctagttgtgaacaaggtgtgaagagcctattgagctacataagaatcctccggcccctgaatgcggctaatcccaacctcggagcaggtggtcacaaaccagtgattggcctgtcgtaacgcgcaagtccgtggcggaaccgactactttgggtgtccgtgtttccttttattttattgtggctgcttatggtgacaatcacagattgttatcataaagcgaattggattggccatccggtgaaagtgagactcattatctatctgtttgctggatccgctccattgagtgtgtttactctaagtacaatttcaacagttatttcaatcagacaattgtatcatatg

SEQ ID NO: 35 - DNA, 730 bp, Artificial Sequence
  source: /note="Description of Artificial Sequence: Synthetic polynucleotide"
cacccactcacagggcccactgggcgctagcactctgattttacgaaatccttgtgcgcctgttttatatcccttccctaattcgaaacgtagaagcaatgcgcaccactgatcaatagtaggcgtaacgcgccagttacgtcatgatcaagcatatctgttcccccggactgagtatcaatagactgcttacgcggttgaaggagaaaacgttcgttatccggctaactacttcgagaagcccagtaacaccatggaagctgcagggtgtttcgctcagcacttcccccgtgtagatcaggtcgatgagccactgcaatccccacaggtgactgtggcagtggctgcgttggcggcctgcctatggggagacccataggacgctctaatgtggacatggtgcgaagagcctattgagctagttagtagtcctccggcccctgaatgcggctaatcctaactgcggagcacatgccttcaacccagagggtagtgtgtcgtaacgggcaactctgcagcggaaccgactactttgggtgtccgtgtttctttttatttttatattggctgcttatggtgacaattacagaattgttaccatatagctattggattggccatccggtgtgtaatagagctgttatatacctatttgttggctttgtaccactaactttaaaatctataactaccctcaactttgtattaactctcaatacagctgatcatg

SEQ ID NO: 36 - DNA, 744 bp, Artificial Sequence
  source: /note="Description of Artificial Sequence: Synthetic polynucleotide"
ttaaaacagctctggggttgtacccaccccagaggcccacgtggcggctagtactccggtattgcggtacccttgtacgcctgttttatactcccttcccgtaacttagacgcacaaaaccaagttcaatagaagggggtacaaaccagtaccaccacgaacaagcacttctgtttccccggtgatgtcgtatagactgcttgcgtggttgaaagcgacggatccgttatccgcttatgtacttcgagaagcccagtaccacctcggaatcttcgatgcgttgcgctcagcactcaaccccagagtgtagcttaggctgatgagtctggacatccctcaccggtgacggtggtccaggctgcgttggcggcctacctatggctaacgccatgggacgctagttgtgaacaaggtgtgaagagcctattgagctacataagaatcctccggcccctgaatgcggctaatcccaacctcggagcaggtggtcacaaaccagtgattggcctgtcgtaacgcgcaagtccgtggcggaaccgactactttgggtgtccgtgtttccttttattttattgtggctgcttatggtgacaatcacagattgttatcataaagcgaattggattggccatccggtgaaagtgagactcattatctatctgtttgctggatccgctccattgagtgtgtttactctaagtacaatttcaacagttatttcaatcagacaattgtatcatatg

SEQ ID NO: 37 - DNA, 20 bp, Artificial Sequence
  source: /note="Description of Artificial Sequence: Synthetic primer"
ttgacggagtcacaccgaat

SEQ ID NO: 38 - DNA, 20 bp, Artificial Sequence
  source: /note="Description of Artificial Sequence: Synthetic primer"
gtcactctgaacaggaggct

(19) United States
(12) Patent Application Publication
(10) Pub. No.: US 2023/0212592 A1
(43) Pub. Date: Jul. 6, 2023

(54) RIBOSWITCH MODULES AND METHODS FOR CONTROLLING EUKARYOTIC PROTEIN TRANSLATION

(71) Applicant: PRESIDENT AND FELLOWS OF HARVARD COLLEGE, Cambridge, MA (US)

(72) Inventors: James J. COLLINS, Cambridge, MA (US); Evan M. ZHAO, Cambridge, MA (US); Xiao TAN, Cambridge, MA (US); Fei RAN, Cambridge, MA (US); Angelo S. MAO, Cambridge, MA (US); Helena DE PUIG GUIXE, Cambridge, MA (US); Emma J. CHORY, Cambridge, MA (US)

(73) Assignee: PRESIDENT AND FELLOWS OF HARVARD COLLEGE, Cambridge, MA (US)

(21) Appl. No.: 18/009,534

(22) PCT Filed: Jun. 11, 2021
(86) PCT No.: PCT/US2021/037030

§ 371 Date: Dec. 9, 2022

Related U.S. Application Data
(60) Provisional application No. 63/038,536, filed on Jun. 12, 2020.

(51) Int. Cl. C12N 15/67; C12N 15/79; C12Q 1/6813; C12Q 1/6876

(52) U.S. Cl. CPC C12N 15/67; C12N 15/79; C12Q 1/6813; C12Q 1/6876

(57) ABSTRACT

The present disclosure provides genetic constructs comprising a recombinant internal ribosome entry site (IRES), which may be used as riboswitches to modulate translation of an operably-mRNA sequence encoding a protein of interest. In other aspects, the disclosure provides recombinant cells, methods, kits and systems that utilize the same, e.g., to provide a platform for modulating the expression of essentially any protein of interest in a eukaryotic cell.

### CROSS-REFERENCE TO RELATED APPLICATIONS

This application claims benefit under 35 U.S.C. § 119(e) of U.S. Provisional Application No. 63/038,536 filed Jun. 12, 2020, the contents of which are incorporated herein by reference in their entirety.

### SEQUENCE LISTING

The instant application contains a Sequence Listing which has been submitted electronically in ASCII format and is hereby incorporated by reference in its entirety. Said ASCII copy, created on Jun. 10, 2021, is named 002806-190120WOPT_SL.txt and is 42,737 bytes in size.

### PARTIES TO A JOINT RESEARCH AGREEMENT

The claimed invention was made by, on behalf of, and/or in connection with one or more of the following parties to a joint research agreement: President and Fellows of Harvard College and BASF Corporation. The joint research agreement was in effect on and before the effective filing date of the claimed invention, and the claimed invention was made as a result of activities undertaken within the scope of the joint research agreement

### TECHNICAL FIELD

The disclosure provides constructs and methods for modulating protein expression in eukaryotic cells using recombinant Group 1 internal ribosome entry site (IRES) elements derived from viral IRES elements.

### BACKGROUND OF THE DISCLOSURE

The regulation of gene expression is critical for growth and development, as well as for the proper maintenance of homeostasis in the face of changing environmental conditions. As such, cells utilize a variety of mechanisms to increase or decrease the production of specific gene products (e.g., proteins or RNA). Expression levels may be modulated, e.g., to trigger developmental pathways, in response to environmental stimuli, or to adapt to new food sources. Gene expression may be modulated at the transcriptional level, e.g., by increasing or decreasing the rate of transcriptional initiation, or aspects of RNA processing. It may also be controlled the post-translational modification of proteins (e.g., by increasing or decreasing the rate of degradation). A myriad of different mechanisms for controlling gene expression exist in nature, and these mechanisms are typically linked to form complex regulatory networks. The use of different mechanisms and triggers permits cells to express specific subsets of genes, or to adjust the level of particular gene products, on an as-needed basis. Doing so conserves energy and resources while also allowing cells to respond more quickly to environmental stimuli. For example, bacteria and eukaryotic cells often adjust the expression of enzyme used in synthetic or metabolic pathways based upon the availability of required substrates or end products. Similarly, many cell types will induce synthesis of protective molecules (e.g., heat shock proteins) in response to environmental stress.

A number of approaches have been developed in order to artificially control levels of gene expression, many of which are modeled on naturally occurring regulatory systems. In general, gene expression can be controlled at the level of RNA transcription or post-transcriptionally, e.g., by controlling the processing or degradation of mRNA molecules, or by controlling their translation. For example, gene expression may be modulated by the administration of small molecule activators or inhibitors (e.g., to increase or decrease the activity of transcription factors), or by the administration of nucleic acids designed to inactivate or degrade mRNA (e.g., using ribozymes, antisense DNA/RNA, and RNA interference techniques). Although these approaches have proven to be useful in many applications, their usefulness may be limited by certain drawbacks. For example, ribozyme, antisense DNA/RNA, and RNAi-based methods normally require a sequence-specific approach (e.g., the small-interfering RNAs used for RNAi and antisense DNA/RNA must be specifically designed for each target). Moreover, the use of small molecule activators and inhibitors to modulate transcription is also non-ideal because such methods typically have a slow response time.

Research efforts to address these shortcomings have led to the development of prokaryotic RNA-sensing modules, referred to as “toehold switches,” which rely on trigger-based unfolding of a ribosome binding site (RBS). See, for example, U.S. Pat. No. 10,208,312, the entire contents of which is hereby incorporated by reference. Toehold switches selectively repress translation of a target transcript by hiding the RBS in the absence of a separate trigger RNA (“trRNA”) and reveal the RBS in the presence of the trRNA, resulting in the initiation of translation of an operably-linked sequence encoding a protein of interest. Prokaryotic toehold switches partially address the shortcoming of other prior art methods by providing an efficient mechanism for modulating translation in prokaryotic organisms. However, this toehold switch mechanism is generally incompatible with eukaryotic systems, which rely on a more complicated set of epigenetic signals to initiate and regulate translation.

### BRIEF SUMMARY OF EXEMPLARY EMBODIMENTS OF THE DISCLOSURE

The present disclosure addresses various needs in the art by providing new genetic constructs and methods for modulation protein translation. These constructs, for example, can be used as a platform to regulate the translation of arbitrary proteins of interest in eukaryotic cells without the need for sequence-specific design modifications. Moreover, the systems described herein allow for the artificial control of gene expression within cells in response to external stimuli.

In particular, the present disclosure describes genetic constructs, recombinant cells, methods, kits and systems that, for example, provide a platform for modulating the expression of essentially any protein of interest in a eukaryotic cell.

In a first general aspect, the present disclosure provides recombinant IRES modules engineered to reduce or prevent translation of an operably-linked mRNA sequence encoding a protein of interest. These recombinant IRES modules are further engineered to fold into an activated form in the presence of a specific trRNA. Once activated, translation of the operably-linked mRNA sequence is allowed to proceed. The trRNA can be an artificial sequence introduced into the cell (e.g., by a plasmid or chemically-mediated transfection) or a sequence found in a naturally-occurring mRNA (e.g., a viral mRNA). As such, these recombinant IRES modules can be used to modulate translation of a protein of interest for therapeutic or industrial applications, and can also be used as a sensor to detect exogenous stimuli, such as a viral infection.

In another general aspect, the disclosure provides a recombinant nucleic acid molecule, comprising: a) a first segment encoding a Group 1 Dicistroviridae internal ribosome entry site (IRES) that has been modified to incorporate exogenous nucleotide sequences at a first site and a second site, and b) a second segment encoding a protein, downstream from and operably linked to the first segment such that translation of the protein is repressed when the IRES is in an inactivated state; wherein the first site comprises a first nucleotide sequence, and the second site comprises a second nucleotide sequence which is the reverse complement of at least a portion of the first nucleotide sequence. In some aspects, the nucleic acid molecule is an mRNA. In some aspects, the second nucleotide sequence is the reverse complement of substantially all of the first nucleotide sequence. In some aspects, the Group 1 Dicistroviridae IRES is a cricket paralysis virus (CrPV), Kashmir bee virus (KBV), or acute bee paralysis virus (ABPV) IRES.

In some aspects, the Group 1 Dicistroviridae IRES has been modified to incorporate exogenous nucleotide sequences at a first site and a second site, wherein the first and second sites are each independently selected from any of Site 1, Site 2, Site 3, Site 4, Site 5, Site 6, Site 7, and Site 8 (Sites 1-8 are defined below and shown in the schematic provided as FIG. 2.). In some aspects, the first and second sites respectively comprise: Site 1 and Site 2, Site 1 and Site 4, Site 1 and Site 5, Site 1 and Site 6, Site 1 and Site 7, Site 1 and Site 8, Site 2 and Site 6, Site 2 and Site 7, Site 4 and Site 6, Site 5 and Site 6, Site 5 and Site 7, Site 6 and Site 7, Site 8 and Site 2, Site 8 and Site 6, or Site 8 and Site 7.

In some aspects, the first nucleotide sequence is 25-80 nt in length. In other aspects, the first nucleotide sequence may have a length within a subrange (e.g., a length of 30-40 nt, 40-50 nt, 50-60 nt, or a length within a subrange defined by any pair of integer values within the range of 25-80 nt). In some aspects, the second nucleotide sequence is 8-25 nt in length. In other aspects, the second nucleotide sequence may have a length within a subrange (e.g., a length of 10-15 nt, 15-25 nt, or a length within a subrange defined by any pair of integer values within the range of 8-25 nt).

In some aspects, the first and second nucleotide sequences are capable of hybridizing when expressed in a eukaryotic cell under in vivo or in vitro conditions, causing the Group 1 Dicistroviridae IRES to fold into an inactivated state.

In still further aspects, the Group 1 Dicistroviridae IRES is configured to fold into an activated state in the presence of a trigger RNA molecule comprising a third nucleotide sequence, wherein the third nucleotide sequence is the reverse compliment of the first nucleotide sequence. The first nucleotide sequence may be capable of hybridizing to the third nucleotide sequence when expressed in a eukaryotic cell under in vivo or in vitro conditions, causing the Group 1 Dicistroviridae IRES to fold into the activated state

In another general aspect, the disclosure provides plasmids and eukaryotic cells encoding any of the recombinant nucleic acid molecules (e.g., any recombinant IRES) described herein. With respect to the eukaryotic cells, it is contemplated that the such recombinant nucleic acid molecules may be incorporated into the genomic or plasmid DNA of the cell. In some aspects, the eukaryotic cell is an animal cell (e.g., a human or primate cell). In some aspects, the eukaryotic cell is not a plant cell.

In another general aspect, the disclosure provides systems and kits that may be used to modulate gene expression in a eukaryotic cell. For instance, the disclosure provides a system for the control of gene expression, comprising: a) a recombinant nucleic acid molecule according to any aspect described herein; and b) a trigger RNA molecule comprising a third nucleotide sequence, wherein the third nucleotide sequence is the reverse compliment of the first nucleotide sequence of the recombinant nucleic acid molecule. Similarly, the disclosure provides kits comprising: a) a plasmid encoding any of the recombinant nucleic acid molecules described herein; and b) a trigger RNA molecule comprising a third nucleotide sequence, wherein the third nucleotide sequence is the reverse compliment of the first nucleotide sequence of the recombinant nucleic acid molecule

In another general aspect, the disclosure provides a recombinant mRNA molecule, comprising: a) a first segment encoding a first protein; b) a second segment, downstream of the first segment, encoding a Group 1 Dicistroviridae IRES that has been modified to incorporate exogenous nucleotide sequences at a first site and a second site; and c) a third segment encoding a second protein, downstream from and operably linked to the second segment such that translation of the second protein is repressed when the IRES is in an inactivated state; wherein transcription of the recombinant mRNA molecule is dependent on RNA Polymerase II, and wherein the first site comprises a first nucleotide sequence, and the second site comprises a second nucleotide sequence which is the reverse complement of at least a portion of the first nucleotide sequence. Such constructs may display reduced translational leakiness compared to other constructs described herein.

In another general aspect, the disclosure provides methods of using the recombinant nucleic acid molecules (e.g., recombinant IRES elements) described herein, in various applications. For example, a method of activating and/or modulating expression of a protein, may comprise: a) providing a eukaryotic cell engineered to express any of the recombinant nucleic acid molecules described herein; b) introducing a trigger RNA molecule comprising a third nucleotide sequence into the eukaryotic cell, wherein the third nucleotide sequence is the reverse compliment of the first nucleotide sequence of the recombinant nucleic acid molecule; wherein the first nucleotide sequence hybridizes to the third nucleotide sequence under in vivo conditions, causing the Group 1 Dicistroviridae IRES to fold into an activated state. In some aspects, the eukaryotic cell engineered to express the recombinant nucleic acid molecule is provided by introducing the recombinant nucleic acid molecule of any of the preceding claims into the eukaryotic cell. The eukaryotic cell used in any of the methods described herein may be, e.g., an animal cell (e.g., a human or primate cell).

As noted above, the recombinant IRES elements described herein may be used as sensors to detect external stimuli. Accordingly, the disclosure provides methods for detecting viral infection of a eukaryotic cell, comprising: a) providing a eukaryotic cell engineered to express the recombinant nucleic acid molecule of any of the preceding claims, wherein the first nucleotide sequence of the recombinant nucleic acid molecule is configured to be the reverse compliment of at least a portion of a mRNA sequence unique to a virus; and b) determining whether the eukaryotic cell is infected with the virus by detecting and/or measuring the presence of the protein encoded by the second segment of the recombinant nucleic acid molecule. The virus may be, e.g., a Dengue virus or a Zika virus.

In still further aspects, the disclosure provides methods for controlling differentiation of a eukaryotic cell, comprising a) providing a eukaryotic cell engineered to express any of the recombinant nucleic acid molecules described herein; and b) culturing the eukaryotic cell; wherein the first nucleotide sequence of the recombinant nucleic acid molecule is configured to be the reverse compliment of at least a portion of a mRNA sequence unique to a selected cell type, and the protein encoded by the second segment of the recombinant nucleic acid molecule comprises a toxin or a protein that causes apoptosis of the selected cell type.

Various exemplary aspects of the presently disclosed inventions are set forth in the accompanying description below. Although methods and materials similar or equivalent to those described herein can be used in the practice or testing of the present invention, illustrative methods and materials are now described. Other features, objects, and advantages of the invention will be apparent from the description and from the claims. In the specification and the appended claims, the singular forms also include the plural unless the context clearly dictates otherwise. Unless defined otherwise, all technical and scientific terms used herein have the same meaning as commonly understood by one of ordinary skill in the art to which this invention belongs.

### BRIEF DESCRIPTION OF THE DRAWINGS

FIG. 1 is a diagram summarizing traditional IRES-mediated eukaryotic gene expression using an unmodified IRES.

FIG. 2 is a schematic representation of a Group 1 CrPV IRES, highlighting the three major Loops (or domains) of this IRES. The architecture of Group 1 IRES elements is conserved among Dicistroviridae family members (e.g., CrPV, KBV, and ABPV).

FIG. 3 is a schematic representation of a Group 1 CrPV IRES, highlighting 8 sites (i.e., “Site 1,” “Site 2,” ... “Site 8”), which can be used as insertion regions for the exogenous nucleic acid sequences described herein.

FIG. 4 is a diagram summarizing eukaryotic gene expression using an exemplary recombinant IRES described herein.

FIG. 5 is a schematic representation of an mRNA construct encoding one of the recombinant IRES elements described herein, as well as a second upstream gene.

FIG. 6 is a graph showing the activity level of different IRES modules. The series, from left to right in FIG. 6 are “+ T7 pol + GFP (Trigger)”, “+T7pol - GFP (Trigger)”, “-T7pol + GFP (Trigger)”, and ““-T7pol - GFP (Trigger).”

FIG. 7 is a graph showing the results of a screen of recombinant IRES riboswitch constructs with a pair of exogenous nucleotide sequence introduced at various sites. The series, from left to right in FIG. 7 are “+ T7 pol + GFP (Trigger)”, “+T7pol - GFP (Trigger)”, “-T7pol + GFP (Trigger)”, and ““-T7pol - GFP (Trigger).”

FIG. 8 is a graph showing the effect of choosing exogenous nucleotide sequences with matching base pairs that break the specified fold and pseudoknot regions. The series, from left to right in FIG. 8 are “+ T7 pol + GFP (Trigger)”, “+T7pol - GFP (Trigger)”, “-T7pol + GFP (Trigger)”, and ““-T7pol - GFP (Trigger).”

FIG. 9 is a graph showing the effect of switching promoters and adding an upstream activation sequence for RNA polymerase I.

FIG. 10 is a graph showing that exemplary recombinant IRES riboswitches described herein are highly specific for their respective trigger RNAs (trRNAs). The series, from left to right in FIG. 10 are “GFP Trigger”, “Azurite Trigger”, and “ySUMO Trigger.”

FIG. 11 is a graph showing the effect of mutations on the functionality of exemplary recombinant IRES riboswitches described herein.

FIG. 12 is a graph showing that recombinant IRES riboswitches may be based on the sequences of IRES modules produced by several Dicistroviridae members (e.g., KBV and ABPV).

FIG. 13 is a diagram showing the use of a recombinant IRES according to the disclosure in a eukaryotic cell as a sensor to detect a viral infection.

FIGS. 14A-14G depict eToehold design and screening. FIG. 14A. eToehold modules are in a locked state in which IRES activity is inhibited, preventing ribosome recruitment and translation. Trigger RNA activates IRES activity through strand invasion and release of the IRES into an activated state, allowing for ribosome binding and protein production. FIG. 14B, Basic screening methodology for eToeholds. Plasmids encoding IRES or eToehold candidates upstream of a reporter protein, polymerase (e.g., T7), and trigger RNA sequence (e.g., GFP) were co-transfected into HEK293T cells. See Example 3 and FIG. 17 for more detailed information. FIG. 14C, Activities of different IRES modules in HEK293T with and without co-transfection with T7 polymerase and GFP trigger sequence. FIG. 14D, Dicistroviridae family IRES structure including three loops critical to translational capability and insertion sites. FIG. 14E, Screen for eToehold modules by inserting two complementary sequences of unequal lengths into insertion sites depicted in FIG. 14D. Numbers denote insertion site of, first, the toehold RNA sequence (~40 base pairs complementary to trigger RNA), and, second, the smaller fragment (~10-18 base pairs) complementary to the first. FIG. 14F, CrPV IRES structure. Regions where insertions overlap with stem loops or pseudoknots (base pair breaking, or BB, sites) are noted. Examples of sequences for the BB sites needed for the CrPV IRES are included for clarity. FIG. 14G, Effect of choosing insertions with matching base pairs (three base pairs) in the regions depicted in FIG. 14D. See Table 1 for construct specifics. All bars represent mean values; dots represent individual data points; error bars represent the s.d. of three experimental replicates exposed to the same conditions. All experiments were repeated at least two times.

FIGS. 15A-15E demonstrate optimization of eToehold expression. FIG. 15A, Effect of switching promoter-polymerase systems and adding an RNA polymerase I upstream activation sequence on expression of eToehold-gated transgene (mKate). FIG. 15B, eToehold activity, as assessed by mKate expression, in the presence of designed trRNA and unmatched RNA. The series, from left to right in FIG. 15B are “GFP Trigger”, “Azurite Trigger”, and “ySUMO Trigger.” FIG. 15C, Schematic of RNA polymerase II-driven eToehold-gated RNA, including stop codons and stem loops. FIG. 15D, eToehold or IRES activity, assessed by mKate expression, of constructs with and without features shown in FIG. 15C. See Table 1 for construct specifics. FIG. 15E, All bar graphs show mean values; dots represent individual data points; error bars represent the s.d. of three experimental replicates exposed to the same conditions. All experiments were repeated at least three times.

FIGS. 16A-16F demonstrate that eToeholds can respond to infection status, cell state, and cell type. FIG. 16A, Stable cell lines were created with eToehold modules designed to sense infection with Zika virus. FIG. 16B, Luminescent signal from cells engineered to express nanoluciferase upon Zika infection after mock, Zika, or Dengue infection. Cells engineered with CrPV-gated nanoluciferase were used as a positive control. FIG. 16C, Stable cell line created with eToehold modules designed to sense exposure to heat by detecting heat shock protein mRNA. FIG. 16D, HeLa cells were transfected with constructs that contained a GFP reporter and eToehold-gated Azurite. FIG. 16E, Constructs designed to translate Azurite protein in the presence of mouse tyrosinase (Tyr) were transfected into B16, D1, or HEK293T (not shown) cells. FIG. 16F, Expression of eToehold-gated Azurite in B16, D1, or HEK293T cells after transfection. All bars show mean values; dots represent individual data points; error bars represent the s.d. of three experimental replicates (four in FIG. 16F) exposed to the same conditions. All experiments were repeated at least three times.

FIG. 17 is a schematic of eToehold screening. HEK293T cells were plated at 60% confluency and transfected with combinations of plasmids encoding T7-polymerase, T7 promoter driving potential eToehold-gated mKate, and trigger construct (GFP). After incubation at 37° C. for 60 hours, cells were detached and analyzed with flow cytometry.

FIGS. 18A-18H depict the gating for flow cytometry experiments. FIGS. 18A-18D depict representative plots for a negative control. FIGS. 18E-18H depict representative plots for a positive GFP and mKate control.

FIGS. 19A-19B demonstrate that eToehold activity dependence on thermodynamics of insertion regions. FIG. 19A, Constructs were based on CrPV eToehold 8-6 designed for GFP sensing. FIG. 19B, constructs were based on CrPV eToehold 6-7 designed for GFP sensing. All data are shown as mean values; dots represent individual data points; error bars represent the s.d. of three experimental replicates exposed to the same conditions. All experiments were repeated at least three times.

FIGS. 20A-20B demonstrate intracellular cytokine staining in cells expressing CrPV IRES with or without additional RNA binding. FIG. 20A, Effects of transfection or transduction with a CrPV IRES construct on production of IL6, CCL5, and CCL2 in primary human fibroblast and muscle skeletal cells. See Table 1 for construct specifics. FIG. 20B, Expression of IL6, CCL5, and CCL2 in cells transduced to express a CrPV IRES construct and “trigger,” compared to those expressing eToehold and an orthogonal nonbinding sequence. Error bars show s.d.; * denote p < 0.05 in 2-way ANOVA. All experiments were repeated at least three times, and dots represent individual data points.

FIGS. 21A-21C are graphs of mean intensity data for presented in FIGS. 14A-14G.

FIGS. 22A-22C demonstrate experiments decreasing basal expression of eToehold modules. FIG. 22A, Constructs with different promoters driving sfGFP were tested based on FIG. 17. FIG. 22B, RNA regions designed to recruit RNA polymerase I factors and decrease 5′ capping were inserted in front of CrPV eToehold 6-7. FIG. 22C, RNA polymerase I responsive promoters were tested based on FIG. 17. See Table 1 for construct specifics. All data are shown as mean values; dots represent individual data points; error bars represent the s.d. of three experimental replicates exposed to the same conditions. Representative flow cytometry plots were chosen within three experimental replicates showing similar results. All experiments were repeated at least two times.

FIGS. 23A-23E are graphs of mean intensity data for FIGS. 15A-15E and FIGS. 16A-16F. The series, from left to right in FIG. 23B are “GFP Trigger”, “Azurite Trigger”, and “ySUMO Trigger.” The series, from left to right in FIG. 23D are “37° C.” and “42° C.” The series, from left to right in FIG. 23E are “B16 (highly expressing Tyr)”, “D1”, and “HEK293T.”

FIG. 24 is a graph demonstrating a decrease of T7 promoter basal expression via insertion of stem loops. Addition of stop codons and stem loops before the eToehold module tested based on FIG. 17. See Table 1 for construct specifics. All data are shown as mean values; dots represent individual data points; error bars represent the s.d. of three experimental replicates exposed to the same conditions. Representative flow cytometry plots were chosen within three experimental replicates showing similar results. All experiments were repeated at least two times.

FIGS. 25A-25B demonstrate eToehold sensitivity to mismatches, and eToeholds based on various IRESs. FIG. 25A, Effect of mismatch mutations within or exterior to the annealing region of inserted RNA sequences on eToehold function. All constructs were based on CrPV eToehold 8-6 designed for GFP sensing. FIG. 25B, eToeholds constructed based on other Dicistroviridae IRESs (namely, KBV and ABPV) retain functionality. See Table 1 for construct specifics. All data are shown as mean values; dots represent individual data points; error bars represent the s.d. of three experimental replicates exposed to the same conditions. All experiments were repeated at least three times.

FIG. 26 is a graph demonstrating that complements to the smaller fragment insertion do not activate eToeholds. EZ-L287, designed for GFP trigger, was tested using the set up in FIG. 17. using different triggers, including a trigger with the reverse compliment of the smaller GFP fragment inserted into ySUMO (see Table 1). All data are shown as mean values; dots represent individual data points; error bars represent the s.d. of three experimental replicates exposed to the same conditions. Experiment was repeated twice.

FIGS. 27A-27B demonstrate that eToeholds function in yeast. eToehold modules gating iRFP were integrated into a yeast strain that expressed GFP (trigger RNA) upon switching of carbon source to galactose. iRFP signal (FIG. 27A) and GFP signal (FIG. 27B) were measured after 6 hours of exponential phase growth. Media was supplemented with biliverdin. See Table 1 for construct specifics. All data are shown as mean values; dots represent individual data points; error bars represent the s.d. of three experimental replicates exposed to the same conditions. Representative flow cytometry plots were chosen within three experimental replicates showing similar results. All experiments were repeated at least two times.

FIGS. 28A-28B demonstrate that eToeholds function in cell-free lysate. FIG. 28A. Wheat germ extract: 50 nM of switch-sfGFP RNA (transcribed from EZ-L214 or EZ-L212 as a control) was added along with different amounts of trigger RNA (transcribed EZ-L366). FIG. 28B. Rabbit reticulocyte lysate: 150 nM of switch-sfGFP RNA (transcribed from EZ-L214 or EZ-L212 as a control) was added along with or without 250 nM of trigger RNA (transcribed EZ-L366). See Table 1 for construct specifics. All data are shown as mean values; error bars represent the s.d. of three experimental replicates exposed to the same conditions. Representative flow cytometry plots were chosen within three experimental replicates showing similar results. All experiments were repeated at least two times.

FIG. 29 is a graph depicting Zika virus concentration sensing with eToeholds, with the same experimental setup as in FIGS. 16A and 16B.

FIGS. 30A-30D demonstrate further viral infection sensing with eToeholds. FIG. 30A, Stable cell line designed for sensing of Zika virus infection contain Zika RNA-responsive eToehold-gated Azurite translation under a T7 promoter. The series, from left to right in FIG. 30A are “Uninfected”, “Infected with Zika Virus”, and “Infected with Dengue Virus.” FIG. 30B, Wildtype Vero E6 cells and stable cell line expressing Zika sensing eToehold gated Azurite as depicted in (FIG. 30A) were subjected to infection with Dengue or Zika virus. Sample gates shown elsewhere. FIG. 30C, Stable cell line designed for sensing of SARS-CoV-2 infection contain SARS-CoV-2 RNA-responsive eToehold-gated Nanoluciferase translation. FIG. 30D Stable cell lines containing eToeholds that sensed different regions of SARS-CoV-2 were transfected with constructs that expressed GFP and two regions of SARS-CoV-2. Luminescence measurements were then taken after furimazine was added. The series, from left to right in FIG. 30D are “Transfected with GFP”, “Transfected with SARS-CoV-2 Spike”, and “Transfected with SARS-CoV-2 3’.” See Table 1 for construct specifics. All bars show mean values; dots represent individual data points; error bars represent the s.d. of three experimental replicates exposed to the same conditions. Representative flow cytometry plots were chosen within three experimental replicates showing similar results. All experiments were repeated at least two times.

FIGS. 31A-31B depict gates used for viral sensing applications. FIG. 31A, Staining results for samples that were infected virus. FIG. 31B, Infection level dependence on MOI of infection as measured by staining and subsequent flow cytometry. Representative flow cytometry plots were chosen within two experimental replicates showing similar results. All experiments were repeated at least two times.

FIGS. 32A-32B depict example gates for Zika virus sensing. Stable cell lines expressing Zika-sensing eToehold gated Azurite (depicted in FIG. 23A) were subjected to infection with Dengue virus and Zika virus. Flow cytometry data are shown based on gates depicted in FIG. 24B, Wildtype Vero E6 cells infected with Zika virus do not exhibit increased Azurite fluorescence.

### DETAILED DESCRIPTION

There exists a need in the art for new constructs that can be used as a platform to regulate the translation of arbitrary proteins of interest in eukaryotic cells without the need for sequence-specific design modifications. Traditional options (e.g., ribozyme, antisense DNA/RNA, and RNAi-based methods) normally require a sequence-specific approach, potentially limiting their usefulness. More recent developments such as prokaryotic toehold switches address some of the shortcomings of traditional options. However, their usefulness is generally confined to prokaryotic organisms due to differences between the translation mechanisms used by prokaryotes and eukaryotes. For example, eukaryotic translation initiation relies on endogenous RNA polymerase II-recruited 5′ modified capping, a poly-adenosine (polyA) tail for mRNA stabilization, and a kozak sequence for protein translational regulation. Although the kozak sequence improves ribosomal binding, it is not an ideal RBS substitute; previously developed kozak-based toeholds have only achieved a maximum two-fold trRNA-driven induction of translation. As such, toehold switches compatible with eukaryotic cells provide limited utility at this time.

In recent years, more complex RNA-based switches have been developed utilizing Cas9 expression and folding of the guide RNA (gRNA). Unfolding of the gRNA leads to activation of the Cas9 enzyme and corresponding repression or activation. Yet, despite bulky circuitry, these mechanisms also induce only modest fold changes (in both eukaryotes and prokaryotes). Similarly, recent advancements in the area of ribozyme research have led to the development of ribozymes that cleave the polyA tail of a target mRNA upon small molecule induction. However, ribozyme-based mechanisms are currently limited to small-length trRNAs, limiting their ability to be tuned for specific sequences, and in any event are limited exclusively to an “ON-to-OFF” sensor, which is non-ideal for leakiness and induction tuning.

The present disclosure addresses these and other shortcomings of known mechanisms for regulating eukaryotic gene expression by providing a minimal component RNA-based sensor (i.e., the recombinant IRES element described herein) that responds “OFF-to-ON” in response to a specific trRNA with a high “ON” vs “OFF” fold change. As such, these constructs are advantageous in expression systems used to produce proteins for industrialor therapeutic use, as well as in other novel applications (e.g., in biosensors capable of detecting environmental stimuli such as the presence of viral mRNA).

### Eukaryotic and Viral Translation Mechanisms

In eukaryotes, protein translation is normally initiated by a tightly-regulated mechanism that requires a modified nucleotide ‘cap’ on the 5′ end of a mRNA, as well as initiation factor proteins (eIFs) that recruit and position the ribosome. In order to bypass this system, many pathogenic viruses use an alternative, cap-independent mechanism that relies upon the use of specific RNA secondary (or tertiary) structures to recruit and manipulate the ribosome, as a substitute for the 5′ cap and eIFs used during the canonical pathway. The RNA elements driving this process are known as IRESs.

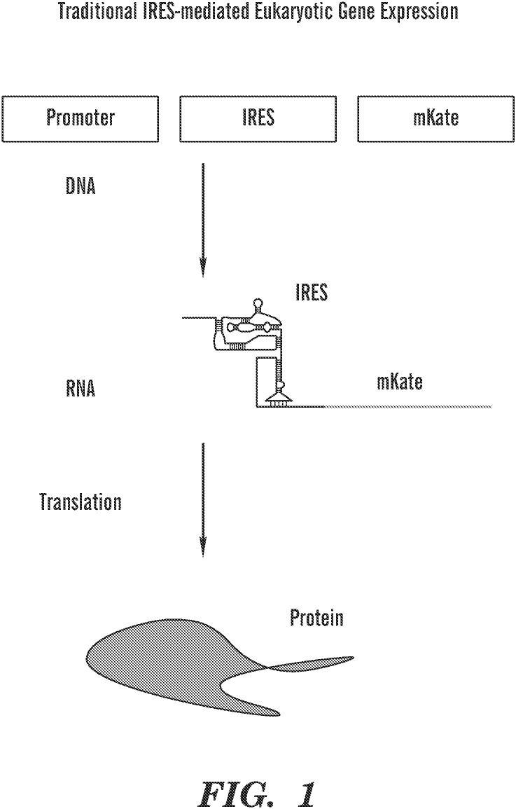

FIG. 1 illustrates the process by which an unmodifid viral IRES can be used to express of an arbitrary protein (in this case, mKate). In brief, traditional IRES-mediated eukaryotic gene expression requires a promoter (e.g., a T7 promoter) operably-linked to a downstream DNA segment encoding a viral IRES and a gene of interest. In this example, the T7 promoter recruits T7 RNA polymerase (which does not 5′ cap mRNA) to transcribe an mRNA comprising the IRES and a segment encoding the protein of interest, i.e., mKate. The viral IRES will normally recruit a ribosome (and potentially other components necessary for translation), resulting in expression of the mKate protein. In this example, the IRES is an unmodified viral IRES rather than a recombinant IRES according to the disclosure, which would be inactive in this example due to the absence of a trRNA.

Viral IRESs have been organized into four distinct groups based on the secondary and tertiary structures of their RNA elements and their mode of action for initiating translation. Within this classification system, Group 1 IRESs are generally more compact and more complex than IRESs in Groups 2-4. Moreover, Group 1 IRESs are notable because they can initiate translation on a non-AUG start codon, do not require any eIFs and do not use the initiator Met-tRNA. Group 1 IRESs are consequently able to promote efficient translation initiation, requiring only the small and large ribosomal subunits. Several Dicistroviridae family members (e.g., cricket paralysis virus (CrPV), Kashmir bee virus (KBV), and acute bee paralysis virus (ABPV)) are known to encode Group 1 IRESs. Group 1 IRESs are highly conserved in terms of sequences, secondary and tertiary structures among Dicistroviridae family members. The CrPV IRES is the most well-studied IRES in this group and is representative of other Group 1 Dicistroviridae IRESs (e.g., of the KBV and ABPV IRESs).

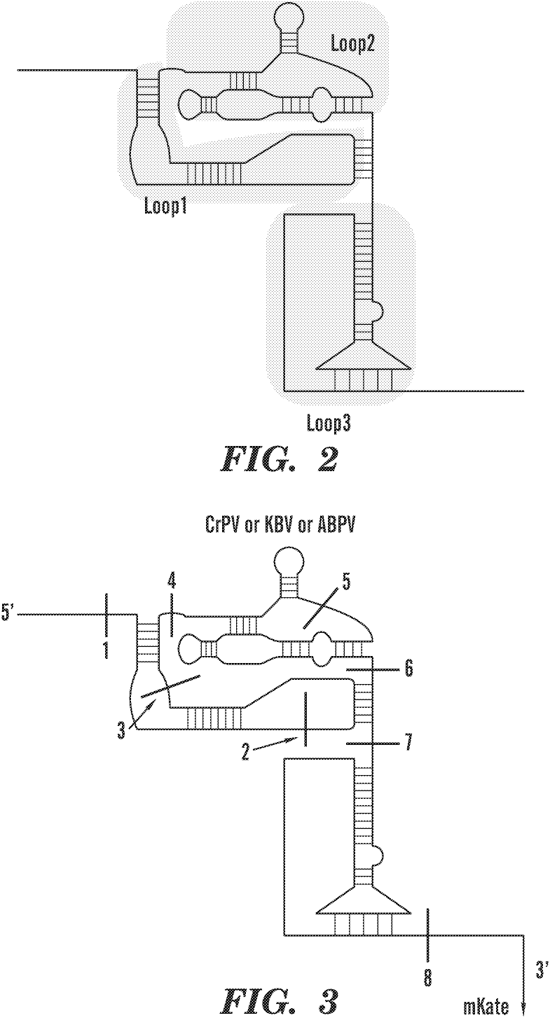

FIG. 2 shows a schematic representation of the secondary structure of the CrPV Group 1 IRES. As illustrated by FIG. 2, the CrPV Group 1 IRES normally folds into a compact structure which has three major loops (or domains) labeled here as Loops 1-3, each including a pseudoknot structure (referred to as PKI, PKII, and PKIII, respectively), as well as internal loops, bulges, and hairpin motifs. This folded structure is essential for IRES activity. For example, the triple-pseudoknot architecture is known to functionally substitute for the initiator met-tRNA during internal initiation, directing translation initiation at a non-AUG triplelet. Normally, the presence of the CrPV Group 1 IRES on a viral mRNA would recruit a eukaryotic ribosome to the mRNA and initiate the translation of the encoded viral protein.

### Recombinant IRES Riboswitches

In some aspects, the present disclosure relates to nucleic acid constructs (e.g., mRNA) which have been modified to incorporate at least one recombinant IRES riboswitch. Embodiments of these recombinant IRES riboswitches can be referred to herein as “eToeholds” or hToeholds.” The recombinant IRES riboswitch can be derived from, or comprises sequences naturally-occurring in a viral IRES. The recombinant IRES can be a viral IRES modified to comprise exogenous, e.g, non-endogenous sequence. In some embodiments of any of the aspects, the recombinant viral IRES comprises a viral IRES comprising two insertions of exogenous, e.g., non-endogenous sequences. In some embodiments of any of the aspects, an insertion in a viral IRES to create a recombinant viral IRES riboswitch described herein can comprise deletion of viral sequences at the insertion site(s). In some embodiments of any of the aspects, an insertion in a viral IRES to create a recombinant viral IRES riboswitch described herein does not comprise deletion of viral sequences at the insertion site(s).

IRES riboswitches described herein can be derived from any IRES sequence obtained or naturally-occurring in a viral genome or sequence. In some embodiments of any of the aspects, the IRES riboswitches described herein can be derived from any IRES sequence obtained or naturally-occurring in a mammalian (e.g., human) pathogenic or mammalian (e.g., human) commensal viral genome or sequence. Such viruses and their sequences are known in the art. In some embodiments of any of the aspects, the IRES sequence can be a Group 1 IRES. In some embodiments of any of the aspects, the IRES sequence can be a Group 1, Group 2, Group 3, or Group 4 IRES. In some embodiments of any of the aspects, the IRES is derived from an IRES sequence of, or the IRES that is modified is an IRES sequence of a Group 1 Discistroviridae IRES; a Hepacivirus IRES; or an Enterovirus IRES. Exemplary wild-type IRES sequences and recombinant IRES riboswitch sequences are provided herein and further wild-type IRES sequences for use in the methods and compositions described herein are readily obtained and/or identified by one of ordinary skill in the art. For example, a database of IRES sequences is available on the world wide web at iresite.org.

In some embodiments of any of the aspects, the Hepacivirus IRES is derived from an IRES sequence of, or the IRES that is modified is an IRES sequence of a hepatitis C virus (HCV); a hepatitis B virus ; a hepatitis F virus; a hepatitis I virus; a hepatitis J virus ; a hepatitis K virus; a hepatitis L virus; a hepatitis M virus; a hepatitis N virus; a Guereza hepacivirus; a hepatitis GB virus B virus; a non-primate hepacivirus NZP1 virus; a Norway rate hepacivirus 1 virus; a Norway rate hepacivirus 2 virus; a bat hepacivirus; a bovine hepacivirus; a equine hepacivirus; a hepacivirus P virus; a rodent hepacivirus; and a wenling shark virus. In some embodiments of any of the aspects, the Hepacivirus IRES is derived from an IRES sequence of, or the IRES that is modified is an IRES sequence of a hepatitis c virus (HCV). Sequences for such viruses are known in the art, e.g., they are available on the world wide web at ncbi.nlm.nih.gov/genomes/GenomesGroup.cgi?taxid=11102.

In some embodiments of any of the aspects, the Enterovirus IRES is derived from an IRES sequence of, or the IRES that is modified is an IRES sequence of a poliovirus (PV); enterovirus 71 (EV71); Enterovirus A virus (e.g., coxsackievirus A2; enterovirus A; or enterovirus A114); Enterovirus B virus (e.g., coxsackievirus B3 or enterovirus B); Enterovirus C; Dromedary camel enterovirus 19CC; Enterovirus D virus (e.g., Enterovirus D or Enterovirus D68); Enterovirus E; Enterovirus F virus (e.g., Enterovirus F or possum enterovirus W1); Enterovirus H virus (e.g., Enterovirus H or simian enterovirus SV4); Enterovirus J virus; Enterovirus SEV-gx; Rhinovirus A virus (e.g., human rhinovirus A1 or rhinovirus A); Rhivnovirus B virus (e.g., human rhinovirus B2 or rhinovirus B14); Rhinovirus C virus (e.g., human rhinovirus NAT001 or rhinovirus C); picornaviridae virus (e.g., picornaviridae sp. Rodent/Ee/PicoV/NX2015); porcine enterovirus (e.g., pocine enterovirus 9); Enterovirus AN12; Enterovirus goat/JL14; Sichuan takin enterovirus; or Yak enterovirus. In some embodiments of any of the aspects, the Enterovirus IRES is derived from an IRES sequence of, or the IRES that is modified is an IRES sequence of a poliovirus (PV) or enterovirus 71 (EV71). Sequences for such viruses are known in the art, e.g., they are available on the world wide web at ncbi.nlm.nih.gov/genomes/GenomesGroup.cgi?taxid=12059.

In some embodiments of any of the aspects, IRES riboswitches described herein are derived from Group I IRES elements used by members of the Dicistroviridae family of viruses (e.g., CrPV, KBV, or ABPV). In some embodiments of any of the aspects, the Group I Discistroviridae IRES is derived from an IRES sequence of, or the IRES that is modified is an IRES sequence of a cricket paralysis virus (CrPV), a Kashmir bee virus (KBV), an acute bee paralysis virus (ABPV), a Plauta Stali Intestine Virus (PSIV) IRES; an aphid lethal paralysis virus (ALPV) IRES; a black queen cell virus (BQCV) IRES; a Drosophila C virus (DCV) IRES; a Himetobi P virus (HiPV) IRES; a Homalodisca coagulata virus-1 (HoCV-1) IRES; a Rhopalosiphum padi virus (RhPV) IRES; or a Triatoma virus (TrV).

As explained above, the naturally-occurring form of these IRES elements recruits ribosomes to an associated mRNA, resulting in translation of the mRNA (i.e., a regulatory circuit that is constitutively active). The co-inventors of the present invention have surprisingly found that Dicistroviridae IRES elements may be genetically modified to produce a recombinant IRES riboswitch that can be switched “ON” or “OFF” based upon the concentration of separate trigger RNA (trRNA) molecule. This new functionality is provided by inserting two or more segments comprising exogenous nucleotide sequences into the original sequence of a viral IRES element. As explained in further detail below, these segments are designed to hybridize in the absence of a corresponding trRNA, causing the recombinant IRES to fold into an inactive state. When the trRNA is provided, hybridization between the two segments is disrupted, allowing the recombinant IRES to fold into a conformation similar to that of the naturally-occurring viral IRESs, which are constitutively active as noted above. The recombinant IRES consequently functions as a riboswitch that can be switched “ON” or “OFF” based upon the concentration of the corresponding trigger RNA, modulating the translation of an operably-linked downstream mRNA sequence encoding a protein of interest.

In some aspects, the IRES riboswitches described herein comprise a nucleotide sequence that shares at least 70% sequence identity with a viral IRES (e.g., a Hepacivirus IRES; or an Enterovirus IRES). In some aspects, the IRES riboswitches described herein display at least 90, 95, 98, 99 or 100% sequence identity with a viral IRES (e.g., a Hepacivirus IRES; or an Enterovirus IRES) at all positions except for the two segments comprising exogenous nucleotide sequences. In some embodiments of any of the aspects, an IRES riboswitch according to the disclosure comprises a nucleotide sequence that shares at least 70, 80, 85, 90, 95, 98, 99 or 100% sequence identity to that of any one of SEQ ID NOs:30-36, except for the presence of exogenous nucleotide sequences inserted at two sites within this sequence, which are indicated by an X in the sequences. In some embodiments of any of the aspects, an IRES riboswitch according to the disclosure comprises a nucleotide sequence with at least 80% sequence identity to any one of SEQ ID NOs:30-36, except for the presence of exogenous nucleotide sequences inserted at two sites within this sequence, which are indicated by an X in the sequences. In some embodiments of any of the aspects, an IRES riboswitch according to the disclosure comprises a nucleotide sequence with at least 85% sequence identity to any one of SEQ ID NOs:30-36, except for the presence of exogenous nucleotide sequences inserted at two sites within this sequence, which are indicated by an X in the sequences. In some embodiments of any of the aspects, an IRES riboswitch according to the disclosure comprises a nucleotide sequence with at least 95% sequence identity to any one of SEQ ID NOs:30-36, except for the presence of exogenous nucleotide sequences inserted at two sites within this sequence, which are indicated by an X in the sequences. The exogenous nucleotide sequences inserted at the two sites can be first and second nucleotide sequences as described elsewhere herein.

In some aspects, the IRES riboswitches described herein comprise a nucleotide sequence that shares at least 70% sequence identity with a Group I Dicistroviridae IRES (e.g., a CrPV, KBV, or ABPV IRES. In some aspects, the IRES riboswitches described herein display at least 90, 95, 98, 99 or 100% sequence identity with a Group I Dicistroviridae IRES at all positions except for the two segments comprising exogenous nucleotide sequences. For example, an IRES riboswitch according to the disclosure may comprise a nucleotide sequence that shares at least 90, 95, 98, 99 or 100% sequence identity to that of SEQ ID NO:1, except for the presence of exogenous nucleotide sequences inserted at two sites within this sequence.

In some embodiments of any of the aspects, the recombinant IRES is derived from an IRES sequence of, or the IRES that is modified is an IRES sequence of a virus other than coxsackievirus B3 (CVB3). In some embodiments of any of the aspects, the recombinant IRES is not derived from an IRES sequence of, or the IRES that is modified is not an IRES sequence of coxsackievirus B3 (CVB3). In some embodiments of any of the aspects, the recombinant IRES is derived from an IRES sequence of, or the IRES that is modified is an IRES sequence of an Enterovirus other than coxsackievirus B3 (CVB3).

In the following explanations of the structure and function of the present recombinant IRES riboswitch technology, reference is made to figures depicting Group 1 Dicistroviridae IRESs and recombinant IRES riboswtiches derived therefrom. These figures are illustrative of exemplary embodiments and do not imply that the technology is limited to these embodiments.

FIG. 3 shows a schematic representation of the CrPV Group 1 IRES, annotated with numeric labels identifying 8 potential insertion sites (Sites 1-8). As noted above, this structure is representative of the structures of other Group 1 Dicistroviridae IRESs (e.g., the KBV and ABPV Group 1 IRESs). These sites shall be referenced herein in various aspects of the disclosure. For example, a recombinant IRES according to the disclosure may comprise an IRES which has a secondary structure that is identical or substantially similar to the secondary structure shown in FIG. 3, but which includes at least one exogenous RNA segment inserted at one or more of Sites 1-8. For example, a recombinant IRES may comprise a sequence derived from the CrPV, KBV, or ABPV viruses, with exogenous segment inserted at Sites 1 and 2, or at Sites 8 and 6, or at any other combination of two or more Sites.

As used herein, the terms “Site 1,” “Site 2,” ... “Site 8” are defined with reference to FIG. 3, which shows a schematic representation of the CrPV Group 1 IRES (which is representative of Group 1 IRESs in members of Dicistroviridae). “Site 1” refers to the region of the IRES that is 5′ to the first stem in Loop 1, whereas “Site 2” refers to the region between the second stem and the pseudoknot (PK1) in Loop 1. “Site 3” refers to the internal loop present between the first and second stems in Loop 1. “Site 4” refers to the region 5′ to the first stem in Loop 2, and “Site 5” refers to the region between the first hairpin and the immediately following stem in Loop 2. “Site 6” refers to the single-stranded region between the last stem of Loop 2 and PK1. “Site 7” similarly refers to the single-stranded region between PK1 and the first stem of Loop 3. Finally, “Site 8” refers to the single-stranded region 3′ to pseudoknot 3 (PK3).

In some embodiments of any of the aspects, the first and second sites respectively comprise: Site 1 and Site 2, Site 1 and Site 8, Site 2 and Site 7, Site 6 and Site 7, or Site 8 and Site 6. In some embodiments of any of the aspects, the first and second sites respectively comprise: Site 6 and Site 7, or Site 8 and Site 6. In some embodiments of any of the aspects, the first and second sites respectively comprise Site 6 and Site 7. In some embodiments of any of the aspects, the first and second sites respectively comprise Site 8 and Site 6. In some aspects, the exogenous nucleotide sequence inserted at one or more of Sites 1-8 may comprise a first nucleotide sequence that is the reverse complement of at least a portion of the nucleotide sequence of a separate trigger RNA molecule. This first nucleotide sequence may be, e.g., 25-80 nt in length. Such constructs may further include a second exogenous nucleotide sequence inserted at a different site selected from Sites 1-8, which comprises a second nucleotide sequence which is the reverse complement of at least a portion of the first nucleotide sequence. This second nucleotide sequence may be, e.g., 8-25 nt in length. In some aspects, this architecture will cause the recombinant IRES to fold into an inactivated state due to interactions between the first and second exogenous nucleotide sequences (e.g., these sequence will at least partially hybridize under in vitro or in vivo conditions due to the second exogenous nucleotide sequence including a segment that is complementary to at least a portion of the first exogenous nucleotide sequence, resulting in attenuation or total loss of the IRES’s ability to initiate translation of an operably-linked protein sequence encoded downstream of the IRES. In some aspects, these constructs may be activated by the presence of the aforementioned trigger RNA molecule, which comprises a nucleotide sequence that is the reverse compliment of the first nucleotide sequence. In some aspects, the trigger RNA molecule may comprise an artificial nucleotide sequence (e.g., to activate translation in an industrial setting), whereas in other aspects this trigger RNA may comprise an endogenous mRNA produced by a eukaryote, prokaryote, or virus (e.g., allowing the IRES to be used as a sensor to detect the presence of a given organism). It is understood that any of the aforementioned nucleotide sequences may consist solely of RNA. However, in some aspects these constructs (e.g., the exogenous nucleotide sequence(s) inserted at one or more of Sites 1-8 and/or the trigger RNA molecule) may include non-RNA or modified RNA bases at one or more positions.

In some aspects, the second exogenous nucleotide sequence is the reverse complement of at least a portion of the first exogenous nucleotide sequence. In some aspects, the first exogenous nucleotide sequence comprises a first nucleotide sequence that is the reverse complement of at least a portion of the nucleotide sequence of a separate trigger RNA molecule. The first nucleotide sequence can be, e.g., 25-80 nt in length, or 40-50 nt in length. The second nucleotide sequence can be, e.g., 8-25 nt or 6-15 nt in length. In some embodiments of any of the aspects, the first nucleotide sequence is 2.5x to 8x longer than the second nucleotide sequence. In some aspects, this architecture will cause the recombinant IRES to fold into an inactivated state due to interactions between the first and second exogenous nucleotide sequences (e.g., these sequence will at least partially hybridize under in vitro or in vivo conditions due to the second exogenous nucleotide sequence including a segment that is complementary to at least a portion of the first exogenous nucleotide sequence, resulting in attenuation or total loss of the IRES’s ability to initiate translation of an operably-linked protein sequence encoded downstream of the IRES. In some aspects, these constructs may be activated by the presence of the aforementioned trigger RNA molecule, which comprises a nucleotide sequence that is the reverse compliment of the first nucleotide sequence. In some aspects, the trigger RNA molecule may comprise an artificial nucleotide sequence (e.g., to activate translation in an industrial setting), whereas in other aspects this trigger RNA may comprise an endogenous mRNA produced by a eukaryote, prokaryote, or virus (e.g., allowing the IRES to be used as a sensor to detect the presence of a given organism). It is understood that any of the aforementioned nucleotide sequences may consist solely of RNA. However, in some aspects these constructs (e.g., the inserted exogenous nucleotide sequence(s) and/or the trigger RNA molecule) may include non-RNA or modified RNA bases at one or more positions.

In some embodiments of any of the aspects, the second nucleotide sequence further comprises an IRES pseudoknot sequence. In some embodiments of any of the aspects, the second nucleotide sequence further comprises an IRES pseudoknot sequence, e.g. a naturally-occurring IRES pseudoknot sequence obtained from a wild-type IRES, including the wild-type IRES being modified as described herein. In some embodiments of any of the aspects, the second nucleotide sequence is inserted into an IRES pseudoknot sequence. IRES pseudoknot structures and sequences are known in the art.

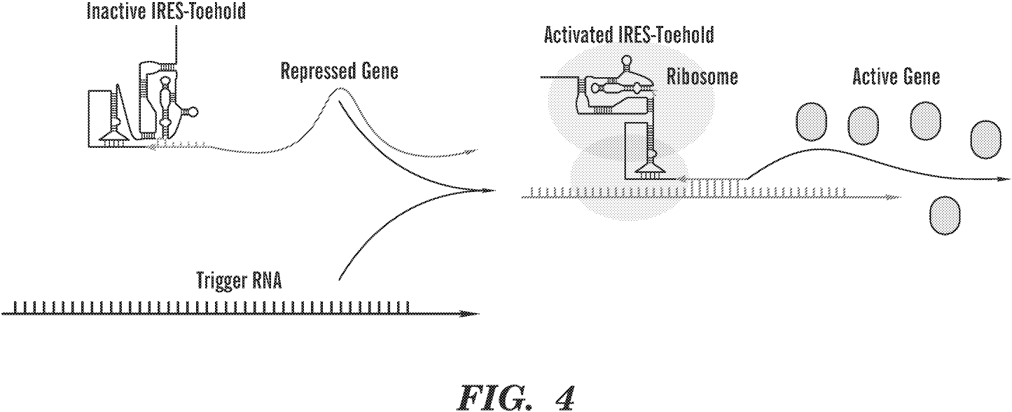

FIG. 4 illustrates the mechanism of operation underlying the recombinant IRES constructs described herein. In this example, an mRNA comprising a recombinant IRES according to the disclosure is shown to be operably-linked to a downstream segment encoding a protein of interest. Translation of the protein of interest is initially repressed because the recombinant IRES includes exogenous sequences at two different insertion sites (i.e., selected from Sites 1-8, defined above) which render the IRES inactive. As explained above, hybridization between the exogenous nucleotide sequences inserted at these sites disrupts the secondary structure of the recombinant IRES (i.e., maintaining the expression switch in the “OFF” state). However, as shown by this figure, when a trRNA is provided, the recombinant IRES switches “ON,” activating translation. The trRNA includes a segment that is a reverse complement of the nucleotide sequence inserted at the first of the two modified sites, and will consequently hybridize with that nucleotide sequence. In doing so, the trRNA disrupts the initial hybridization between the two exogenous nucleotide sequences, allowing the recombinant IRES to refold into an activated state.

In some aspects, recombinant IRES constructs according to the disclosure are incorporated into mRNA transcripts produced by a T7 RNA polymerase (e.g., such constructs may be downstream of and operably-linked to a T7 promoter sequence). The T7 polymerase may be produced by the eukaryotic cell (e.g., expressed from genomic DNA of the cell or from a plasmid) or introduced into the eukaryotic cell. In other aspects described herein, the recombinant IRES construct may be incorporated into an mRNA transcript produced by an alternative polymerase (e.g., eukaryotic RNA polymerase II).

As noted above, the recombinant IRES constructs described herein may be incorporated into mRNA transcripts produced by a viral RNA polymerase (e.g., T7 polymerase, which does not apply a 5′ cap) because these constructs are able to recruit a ribosome and initiate translation. However, in some cases it may be undesirable to use a viral polymerase (e.g., a host cell may not produce T7, requiring co-transfection with a vector to supply this enzyme). Alternatively, it may be desirable to use endogenous RNA polymerase II for transcription in order to design a riboswitch system that uses a reduced number of exogenous components.

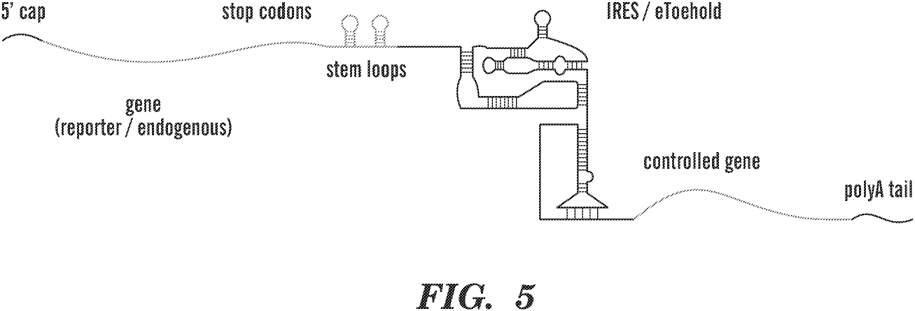

FIG. 5 is a schematic representation of an mRNA produced by RNA polymerase II which incorporates a recombinant IRES according to the disclosure. A illustrated by this schematic, the mRNA comprises a segment encoding a first protein, followed by a set of stop codons. A recombinant IRES according to the disclosure is present downstream from this element, and operably-linked to a segment encoding a second protein. Note that the mRNA transcript has a 5′ cap and a polyA tail, resulting from transcription by RNA polymerase II in this case. Translation of the second protein is controlled by the recombinant IRES, as is the case with constructs according to other aspects described herein. However, this configuration may be preferable in some instances due to its reliance on an endogenous mammalian mRNA promoter and polymerase, rather than viral components. Furthermore, as discussed in the examples below, this configuration appears to display reduced leakiness of expression compared to exemplary aspects which omit the upstream gene of interest.

Accordingly, in one aspect of any of the embodiments, described herein is a recombinant mRNA molecule, comprising: a first segment encoding a first protein; a second segment, downstream of the first segment, encoding a recombinant viral internal ribosome entry site (IRES) that has been modified to incorporate exogenous nucleotide sequences at a first site and a second site; and a third segment encoding a second protein, downstream from and operably linked to the second segment such that translation of the second protein is repressed when the IRES is in an inactivated state; wherein transcription of the recombinant mRNA molecule is dependent on a polymerase, and wherein the first site comprises a first nucleotide sequence, and the second site comprises a second nucleotide sequence which is the reverse complement of at least a portion of the first nucleotide sequence.

In various embodiments of any of the aspects, a nucleic acid sequence encoding a protein, located either 5′ or 3′ of a recombinant viral IRES riboswitch described herein can encode a protein which is a reporter protein, e.g., which produces a detectable signal. A reporter protein is a polypeptide with an easily assayed enzymatic activity or detectable signal that is naturally absent from the host cell. Exemplary but non-limiting reporter proteins include lacZ, catalase, xylE, GFP, RFP, YFP, ySUMO, CFP, EYFP, ECFP, mRFP1, mOrange, GFPmut3b, OFP, mBanana, neomycin phosphotransferase, luciferase, mCherry, and derivatives or variants thereof. In some embodiments of any of the aspects, the reporter protein is suitable for use in a colorimetric, luminescence, or fluorescence assay.

The recombinant IRES riboswitches described herein can be used as sensor modules, e.g., to detect particular trRNAs. The recombinant IRES riboswitches can be designed such that the trRNA is a sequence present in a target eukaryotic organism, target prokaryotic organism, or target virus. In the presence of the target organism/virus, or the target organism/virus in a particular transcriptional state, the recombinant IRES riboswitch will assume an active state and the protein encoded 3′ of the modified IRES sequence (e.g., a reporter protein) will be expressed, indicating the presence of the target. Such sensor systems are demonstrated herein, e.g., in Example 3 where infection with a number of different viruses is detected. In some embodiments, the target prokaryotic organism or target virus is a pathogen, e.g., a mammalian or human pathogen. In some embodiments, the target virus is Zika virus, Dengue virus, or a coronavirus (e.g., SARS-CoV-2). In some embodiments, the target prokaryotic or eukaryotic organism can be an organism can be an organism comprising and/or expressing the recombinant IRES riboswitches and the trRNA can be a non-constitutively expressed RNA, e.g., an RNA expressed only at certain developmental or differentiation stages, or a RNA expressed in response to certain stimuli and/or stresses.

### Recombinant Cells Engineered to Incorporate Riboswitch Modules

In some aspects, the disclosure provides eukaryotic cells, other than plant cells, engineered to express proteins under the control of the recombinant IRESs described herein.

The eukaryotic cell may be a animal, fungal, or protist cell. In some aspects, the eukaryotic cell may comprise genomic DNA encoding a recombinant IRES. In others, the recombinant IRES may be encoded by a vector (e.g., a plasmid) present within the eukaryotic cell. The recombinant IRES may be operably-linked to an endogenous or exogenous promoter and/or a gene encoding a protein of interest.

### Use of Riboswitch Modules in Cell-Free Expression Systems

In some aspects, the recombinant IRES modules described herein may be used in cell-free expression systems. For example, a kit or assay may utilize a cell-free lysate produced from eukaryotic cells that includes DNA encoding at least one mRNA which incorporates a recombinant IRES module. In other aspects, such kits or assays may include transcribed mRNAs that incorporate at least one recombinant IRES module. It is understood that that the riboswitch mechanism described herein may be used as a sensor to trigger expression of a protein of interest in a variety of in vitro applications (e.g., as a sensor to detect the presence of viral mRNA).

### Modulating Translation in Eukaryotes or Cell-Free Expression Systems Using Recombinant IRES Riboswitch Modules

The recombinant IRES riboswitch modules described herein may be used to modulate the expression of a protein of interest, e.g., in a eukaryotic cell or in a cell-free expression system.

In some aspects, a eukaryotic cell may be transfected with a vector that encodes a protein of interest operably-linked to an upstream IRES riboswitch according to the disclosure. The IRES riboswitch may comprise a sequence sharing at least 90, 95, 98, 99 or 100% sequence identity with that of a viral IRES, except for the presence of exogenous nucleotide sequences at two sites. In some embodiments, the IRES riboswitch may comprise a sequence sharing at least 90, 95, 98, 99 or 100% sequence identity with that of a Group I Dicistroviridae IRES (e.g., the CrPV IRES represented by SEQ ID NO: 1), except for the presence of exogenous nucleotide sequences at two sites (e.g., any combination of Sites 1-8, as defined above). This pair of exogenous sequences may comprise a first nucleotide sequence that is 25-80 nt in length and a second nucleotide sequence that is 8-25 nt in length, wherein second nucleotide sequence is the reverse complement of a portion of the first nucleotide sequence, causing the pair of exogenous sequences to hybridize. As a result of this hybridization, the IRES riboswitch assumes an inactive fold, preventing translation of the downstream protein of interest. Translation may be activated by introducing a trRNA which comprises a nucleotide sequence that is the reverse complement of the first nucleotide sequence, causing the first nucleotide sequence to hybridize with the trRNA rather than the second nucleotide sequence, and consequently allowing the IRES riboswitch to assume an active fold. In some aspects, the trRNA may be introduced by transfection or expressed by a vector.

In some aspects, the trRNA may be configured to have a unique sequence that is not found in mRNAs expressed by the eukaryotic cell used for expression. The selection of a unique sequence may reduce or eliminate off-target effects (e.g., unintended hybridization between the trRNA and other endogenous mRNAs produced by the eukaryotic cell).

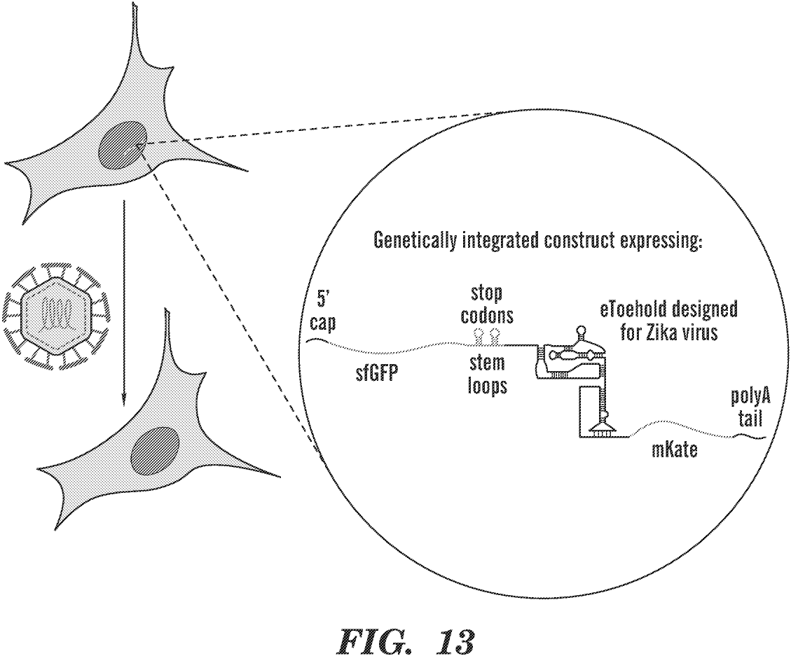

In some aspects, the trRNA may comprise a portion of an mRNA expressed by the eukaryotic cell or an external stimulus (e.g., a viral mRNA produced following infection of the cell by a virus, as shown by FIG. 13). In some aspects, the concentration of the trRNA may be increased or decreased to modulate expression of the protein of interest.

The mRNA comprising the IRES riboswitch may be operably-linked to a promoter suitable for expression in the selected eukaryotic cell. In some aspects, a T7 promoter may be used (e.g., if the selected eukaryotic cell is engineered to produce T7 polymerase). Alternatively, a eukaryotic promoter (e.g., an RNA Polymerase II promoter) may be used. The selection of a suitable promoter will vary depending on the intended application of the IRES riboswitches described herein. For example, an inducible promoter may be desirable in some applications, whereas a constitutive promoter may be desired in others. Some promoters may also allow for tighter control over expression of the mRNA (e.g., a T7 promoter may be leaky when used in a eukaryotic cell due to low-level recruitment of RNA polymerase II).

In some embodiments of any of the aspects, the IRES riboswitch can be operably-linked to one or more of: a) an IRES pseudoknot sequence; b) an IRES pseudoknot sequence found in the wild-type sequence of a virus in which the IRES naturally occurs; c) a promoter and/or upstream activating factor binding sequence; d) a stop codon; e) a stem-loop (e.g., SEQ ID NO: 10); f) a 5′ cap; g) a reporter gene; and h) a poly-A tail, wherein the one or more of elements a-h are individually located 5′ or 3′ of the IRES riboswitch. In some embodiments of any of the aspects, the IRES riboswitch can be operably-linked to one or more of: a) an IRES pseudoknot sequence; b) an IRES pseudoknot sequence found in the wild-type sequence of a virus in which the IRES naturally occurs; c) a promoter and/or upstream activating factor binding sequence; d) a stop codon; e) a stem-loop; f) a 5′ cap; and g) a reporter gene; wherein the one or more of elements a-g are individually located 5′ of the IRES riboswitch.

A promoter for use in the methods and compositions described herein can be an RNA polymerase II; a polymerase other than RNA polymerase II; a T7 polymerase; a T3 promoter, a araBAD promoter, a trp promoter, a lac promoter, a Ptac promoter, a pL promoter, and/or an SP6 polymerase. An upstream activating factor binding sequence can be the upstream activation factor binding DNA sequence (UAF2) from Saccharomyces cerevisiae (e.g., SEQ ID NO: 11).

In one aspect of any of the embodiments, described herein is a vector, e.g., a plasmid or viral vector comprising the recombinant nucleic acid molecule, expression construct, recombinant mRNA molecule, or recombinant IRES riboswitch described herein. In one aspect of any of the embodiments, described herein is a eukaryotic cell (e.g., an animal cell, human cell, or primate cell) comprising DNA encoding a recombinant nucleic acid molecule, expression construct, or recombinant mRNA molecule described herein, wherein the eukaryotic cell is not a plant cell and the DNA is: integrated into the genomic DNA of the eukaryotic cell, or present on a vector (e.g, a plasmid or viral vector) present within the eukaryotic cell. In one aspect of any of the embodiments, described herein is a prokaryotic cell comprising DNA encoding a recombinant nucleic acid molecule, expression construct, or recombinant mRNA molecule described herein, wherein the DNA is: integrated into the genomic DNA of the prokaryotic cell, or present on a vector (e.g, a plasmid or viral vector) present within the prokaryotic cell. Such cells are considered to be engineered by the introduction of the recombinant nucleic acid molecule, expression construct, recombinant mRNA molecule, or recombinant IRES riboswitch and can be used in methods of activating and/or modulating expression of a protein comprising providing the providing a eukaryotic cell engineered to express the recombinant nucleic acid molecule of any of the preceding claims; and introducing a trigger RNA molecule comprising a third nucleotide sequence into the eukaryotic cell, wherein the third nucleotide sequence is the reverse compliment of the first nucleotide sequence of the recombinant nucleic acid molecule; wherein the first nucleotide sequence hybridizes to the third nucleotide sequence under in vivo conditions, causing the recombinant IRES riboswitch to fold into an activated state, further wherein the eukaryotic cell is not a plant cell.

It is understood that the IRES riboswitches described herein may also be used in cell-free expressions. For example, a kit may include mRNA comprising an IRES riboswitch operably linked to a segment encoding a protein of interest, as well as components needed for in vitro protein expression (e.g., a cellular lysate).

The IRES riboswitches described herein may be used in a variety of therapeutic and industrial, applications. For example, a subject may be administered a gene therapy, wherein nucleic acids are introduced into the subject’s cells which express a therapeutic protein under the control of an IRES riboswitch. The level of expression of the protein may be modulated by administration of a trRNA to the patient. IRES riboswitches may also be used in the laboratory or medical field as a means to control cell differentiation. For example, a stem cell may be engineered to incorporate an IRES riboswitch triggered by an mRNA produced by a specific cell type, wherein the riboswitch controls expression of a toxin or a protein that induces apoptosis. Such mechanisms may be used to maintain the purity of a stem cell line by eliminating undesirable cell types which may be produced inadvertently.

In another embodiment, the IRES riboswitches described herein can be used in a method of detecting viral infection of a cell. For example, a eukaryotic cell is engineered to express a recombinant nucleic acid comprising an IRES riboswitch as described herein, wherein the first nucleotide sequence of the recombinant nucleic acid molecule is configured to be the reverse compliment of at least a portion of a mRNA sequence unique to a virus; and it is determined whether the eukaryotic cell is infected with the virus by detecting and/or measuring the presence of the protein encoded by the second segment of the recombinant nucleic acid molecule, wherein the eukaryotic cell is not a plant cell. The virus can be, e.g., Dengue virus, Zika virus, or a coronavirus.

In another embodiment, the IRES riboswitches described herein can be used in a method of controlling or monitoring differentiation of a eukaryotic cell. For example, a eukaryotic cell is engineered to express a recombinant nucleic acid comprising an IRES riboswitch as described herein, and the cell is cultured, wherein the first nucleotide sequence of the recombinant nucleic acid molecule is configured to be the reverse compliment of at least a portion of a mRNA sequence unique to a selected cell type, and the protein encoded by the second segment of the recombinant nucleic acid molecule comprises a toxin or a protein that causes apoptosis of the selected cell type, further wherein the eukaryotic cell is not a plant cell.

In some aspects of any of the embodiments, described herein is a kit or system, comprising one or more of: a plasmid or viral vector, a recombinant nucleic acid molecule, expression construct, recombinant mRNA molecule, recombinant IRES riboswitch, and/or trRNA as described herein. A kit is an assemblage of materials or components, including at least one of the foregoing elements described herein. The exact nature of the components configured in the kit depends on its intended purpose. In some embodiments of any of the aspects, a kit includes instructions for use. “Instructions for use” typically include a tangible expression describing the technique to be employed in using the components of the kit, e.g., to detect an organism or RNA. Still in accordance with the present invention, “instructions for use” may include a tangible expression describing the preparation of a recombinant IRES riboswitch, cell, or expression system described herein such as reconstitution, dilution, mixing, or incubation instructions, and the like, typically for an intended purpose. Optionally, the kit also contains other useful components, such as, measuring tools, diluents, buffers, syringes, pharmaceutically acceptable carriers, or other useful paraphernalia as will be readily recognized by those of skill in the art.

The materials or components assembled in the kit can be provided to the practitioner stored in any convenient and suitable ways that preserve their operability and utility. For example, the components can be in dissolved, dehydrated, or lyophilized form; they can be provided at room, refrigerated or frozen temperatures. The components are typically contained in suitable packaging material(s). As employed herein, the phrase “packaging material” refers to one or more physical structures used to house the contents of the kit, such as inventive compositions and the like. The packaging material is constructed by well-known methods, preferably to provide a sterile, contaminant-free environment. The packaging may also preferably provide an environment that protects from light, humidity, and oxygen. As used herein, the term “package” refers to a suitable solid matrix or material such as glass, plastic, paper, foil, polyester (such as polyethylene terephthalate, or Mylar) and the like, capable of holding the individual kit components. Thus, for example, a package can be a glass vial used to contain suitable quantities of a composition described herein. The packaging material generally has an external label which indicates the contents and/or purpose of the kit and/or its components.

For convenience, the meaning of some terms and phrases used in the specification, examples, and appended claims, are provided below. Unless stated otherwise, or implicit from context, the following terms and phrases include the meanings provided below. The definitions are provided to aid in describing particular embodiments, and are not intended to limit the claimed invention, because the scope of the invention is limited only by the claims. Unless otherwise defined, all technical and scientific terms used herein have the same meaning as commonly understood by one of ordinary skill in the art to which this invention belongs. If there is an apparent discrepancy between the usage of a term in the art and its definition provided herein, the definition provided within the specification shall prevail.

For convenience, certain terms employed herein, in the specification, examples and appended claims are collected here.

The terms “decrease”, “reduced”, “reduction”, or “inhibit” are all used herein to mean a decrease by a statistically significant amount. In some embodiments, “reduce,” “reduction” or “decrease” or “inhibit” typically means a decrease by at least 10% as compared to a reference level (e.g. the absence of a given treatment or agent) and can include, for example, a decrease by at least about 10%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, at least about 98%, at least about 99% , or more. As used herein, “reduction” or “inhibition” does not encompass a complete inhibition or reduction as compared to a reference level. “Complete inhibition” is a 100% inhibition as compared to a reference level.

The terms “increased”, “increase”, “enhance”, or “activate” are all used herein to mean an increase by a statically significant amount. In some embodiments, the terms “increased”, “increase”, “enhance”, or “activate” can mean an increase of at least 10% as compared to a reference level, for example an increase of at least about 20%, or at least about 30%, or at least about 40%, or at least about 50%, or at least about 60%, or at least about 70%, or at least about 80%, or at least about 90% or up to and including a 100% increase or any increase between 10-100% as compared to a reference level, or at least about a 2-fold, or at least about a 3-fold, or at least about a 4-fold, or at least about a 5-fold or at least about a 10-fold increase, or any increase between 2-fold and 10-fold or greater as compared to a reference level.

As used herein, the terms “protein” and “polypeptide” are used interchangeably herein to designate a series of amino acid residues, connected to each other by peptide bonds between the alpha-amino and carboxy groups of adjacent residues. The terms “protein”, and “polypeptide” refer to a polymer of amino acids, including modified amino acids (e.g., phosphorylated, glycated, glycosylated, etc.) and amino acid analogs, regardless of its size or function. “Protein” and “polypeptide” are often used in reference to relatively large polypeptides, whereas the term “peptide” is often used in reference to small polypeptides, but usage of these terms in the art overlaps. The terms “protein” and “polypeptide” are used interchangeably herein when referring to a gene product and fragments thereof. Thus, exemplary polypeptides or proteins include gene products, naturally occurring proteins, homologs, orthologs, paralogs, fragments and other equivalents, variants, fragments, and analogs of the foregoing.

As used herein, the term “nucleic acid” or “nucleic acid sequence” refers to any molecule, preferably a polymeric molecule, incorporating units of ribonucleic acid, deoxyribonucleic acid or an analog thereof. The nucleic acid can be either single-stranded or double-stranded. A single-stranded nucleic acid can be one nucleic acid strand of a denatured double- stranded DNA. Alternatively, it can be a single-stranded nucleic acid not derived from any double-stranded DNA. In one aspect, the nucleic acid can be DNA. In another aspect, the nucleic acid can be RNA. Suitable DNA can include, e.g., genomic DNA or cDNA. Suitable RNA can include, e.g., mRNA.

The term “expression” refers to the cellular processes involved in producing RNA and proteins and as appropriate, secreting proteins, including where applicable, but not limited to, for example, transcription, transcript processing, translation and protein folding, modification and processing. Expression can refer to the transcription and stable accumulation of sense (mRNA) or antisense RNA derived from a nucleic acid fragment or fragments of the invention and/or to the translation of mRNA into a polypeptide.

In some embodiments, the expression of nucleic acid sequence and/or protein described herein is/are tissue-specific. In some embodiments, the expression of a nucleic acid sequence and/or protein described herein is/are global. In some embodiments, the expression of a nucleic acid sequence and/or protein described herein is systemic.

“Expression products” include RNA transcribed from a gene, and polypeptides obtained by translation of mRNA transcribed from a gene. The term “gene” means the nucleic acid sequence which is transcribed (DNA) to RNA in vitro or in vivo when operably linked to appropriate regulatory sequences. The gene may or may not include regions preceding and following the coding region, e.g. 5′ untranslated (5′UTR) or “leader” sequences and 3′ UTR or “trailer” sequences, as well as intervening sequences (introns) between individual coding segments (exons).

“Operably linked” refers to an arrangement of elements wherein the components so described are configured so as to perform their usual function. Thus, control elements operably linked to a coding sequence are capable of effecting the expression of the coding sequence. The control elements need not be contiguous with the coding sequence, so long as they function to direct the expression thereof. Thus, for example, intervening untranslated yet transcribed sequences can be present between a promoter sequence and the coding sequence and the promoter sequence can still be considered “operably linked” to the coding sequence.

In some embodiments, the methods described herein relate to measuring, detecting, or determining the level of at least one target. As used herein, the term “detecting” or “measuring” refers to observing a signal from, e.g. a probe, label, or target molecule to indicate the presence of an analyte in a sample. Any method known in the art for detecting a particular label moiety can be used for detection. Exemplary detection methods include, but are not limited to, spectroscopic, fluorescent, photochemical, biochemical, immunochemical, electrical, optical or chemical methods. In some embodiments of any of the aspects, measuring can be a quantitative observation.

In some embodiments of any of the aspects, a polypeptide, nucleic acid, or cell as described herein can be engineered. As used herein, “engineered” refers to the aspect of having been manipulated by the hand of man. For example, a polypeptide is considered to be “engineered” when at least one aspect of the polypeptide, e.g., its sequence, has been manipulated by the hand of man to differ from the aspect as it exists in nature. As is common practice and is understood by those in the art, progeny of an engineered cell are typically still referred to as “engineered” even though the actual manipulation was performed on a prior entity.

The term “exogenous” refers to a substance present in a cell or nucleic acid sequence other than its native source. The term “exogenous” when used herein can refer to a nucleic acid (e.g. a nucleic acid encoding a polypeptide) or a polypeptide that has been introduced by a process involving the hand of man into a nucleic acid molecule or biological system such as a cell or organism in which it is not normally found and one wishes to introduce the nucleic acid or polypeptide into such a nucleic acid molecule, cell or organism. In contrast, the term “endogenous” refers to a substance that is native to the nucleic acid molecule or biological system or cell.

In some embodiments, a nucleic acid as described herein is comprised by a vector. In some of the aspects described herein, a nucleic acid sequence as described herein, or any module thereof, is operably linked to a vector. The term “vector”, as used herein, refers to a nucleic acid construct designed for delivery to a host cell or for transfer between different host cells. As used herein, a vector can be viral or non-viral. The term “vector” encompasses any genetic element that is capable of replication when associated with the proper control elements and that can transfer gene sequences to cells. A vector can include, but is not limited to, a cloning vector, an expression vector, a plasmid, phage, transposon, cosmid, chromosome, virus, virion, etc.

In some embodiments of any of the aspects, the vector or nucleic acid is recombinant, e.g., it comprises sequences originating from at least two different sources. In some embodiments of any of the aspects, the vector or nucleic acid comprises sequences originating from at least two different species. In some embodiments of any of the aspects, the vector nucleic acid comprises sequences originating from at least two different genes. A sequence can be modified to be recombinant, or a sequence can be integrated into another sequence to provide a recombinant sequence by methods well known in the art, e.g., through use of restriction enzymes and ligases.

In some embodiments of any of the aspects, the vector or nucleic acid described herein is codon-optimized, e.g., the native or wild-type sequence of the nucleic acid sequence has been altered or engineered to include alternative codons such that altered or engineered nucleic acid encodes the same polypeptide expression product as the native/wild-type sequence, but will be transcribed and/or translated at an improved efficiency in a desired expression system. In some embodiments of any of the aspects, the expression system is an organism other than the source of the native/wild-type sequence (or a cell obtained from such organism). In some embodiments of any of the aspects, the vector and/or nucleic acid sequence described herein is codon-optimized for expression in a mammal or mammalian cell, e.g., a mouse, a murine cell, or a human cell. In some embodiments of any of the aspects, the vector and/or nucleic acid sequence described herein is codon-optimized for expression in a human cell. In some embodiments of any of the aspects, the vector and/or nucleic acid sequence described herein is codon-optimized for expression in a yeast or yeast cell. In some embodiments of any of the aspects, the vector and/or nucleic acid sequence described herein is codon-optimized for expression in a bacterial cell. In some embodiments of any of the aspects, the vector and/or nucleic acid sequence described herein is codon-optimized for expression in an E. coli cell.

As used herein, the term “expression vector” refers to a vector that directs expression of an RNA or polypeptide from sequences linked to transcriptional regulatory sequences on the vector. The sequences expressed will often, but not necessarily, be heterologous to the cell. An expression vector may comprise additional elements, for example, the expression vector may have two replication systems, thus allowing it to be maintained in two organisms, for example in human cells for expression and in a prokaryotic host for cloning and amplification.

As used herein, the term “viral vector” refers to a nucleic acid vector construct that includes at least one element of viral origin and has the capacity to be packaged into a viral vector particle. The viral vector can contain the nucleic acid encoding a polypeptide as described herein in place of non-essential viral genes. The vector and/or particle may be utilized for the purpose of transferring any nucleic acids into cells either in vitro or in vivo. Numerous forms of viral vectors are known in the art.

It should be understood that the vectors described herein can, in some embodiments, be combined with other suitable compositions and therapies. In some embodiments, the vector is episomal. The use of a suitable episomal vector provides a means of maintaining the nucleotide of interest in the subject in high copy number extra chromosomal DNA thereby eliminating potential effects of chromosomal integration.

As used herein, “contacting” refers to any suitable means for delivering, or exposing, an agent to at least one cell. Exemplary delivery methods include, but are not limited to, direct delivery to cell culture medium, perfusion, injection, or other delivery method well known to one skilled in the art. In some embodiments, contacting comprises physical human activity, e.g., an injection; an act of dispensing, mixing, and/or decanting; and/or manipulation of a delivery device or machine.

The term “statistically significant” or “significantly” refers to statistical significance and generally means a two standard deviation (2SD) or greater difference.

Other than in the operating examples, or where otherwise indicated, all numbers expressing quantities of ingredients or reaction conditions used herein should be understood as modified in all instances by the term “about.” The term “about” when used in connection with percentages can mean ±1%.

As used herein, the term “comprising” means that other elements can also be present in addition to the defined elements presented. The use of “comprising” indicates inclusion rather than limitation.

The term “consisting of” refers to compositions, methods, and respective components thereof as described herein, which are exclusive of any element not recited in that description of the embodiment.

As used herein the term “consisting essentially of” refers to those elements required for a given embodiment. The term permits the presence of additional elements that do not materially affect the basic and novel or functional characteristic(s) of that embodiment of the invention.

As used herein, the term “corresponding to” refers to an amino acid or nucleotide at the enumerated position in a first polypeptide or nucleic acid, or an amino acid or nucleotide that is equivalent to an enumerated amino acid or nucleotide in a second polypeptide or nucleic acid. Equivalent enumerated amino acids or nucleotides can be determined by alignment of candidate sequences using degree of homology programs known in the art, e.g., BLAST.

The singular terms “a,” “an,” and “the” include plural referents unless context clearly indicates otherwise. Similarly, the word “or” is intended to include “and” unless the context clearly indicates otherwise. Although methods and materials similar or equivalent to those described herein can be used in the practice or testing of this disclosure, suitable methods and materials are described below. The abbreviation, “e.g.” is derived from the Latin exempli gratia, and is used herein to indicate a non-limiting example. Thus, the abbreviation “e.g.” is synonymous with the term “for example.”

Groupings of alternative elements or embodiments of the invention disclosed herein are not to be construed as limitations. Each group member can be referred to and claimed individually or in any combination with other members of the group or other elements found herein. One or more members of a group can be included in, or deleted from, a group for reasons of convenience and/or patentability. When any such inclusion or deletion occurs, the specification is herein deemed to contain the group as modified thus fulfilling the written description of all Markush groups used in the appended claims.

Unless otherwise defined herein, scientific and technical terms used in connection with the present application shall have the meanings that are commonly understood by those of ordinary skill in the art to which this disclosure belongs. It should be understood that this invention is not limited to the particular methodology, protocols, and reagents, etc., described herein and as such can vary. The terminology used herein is for the purpose of describing particular embodiments only, and is not intended to limit the scope of the present invention, which is defined solely by the claims. Definitions of common terms in immunology and molecular biology can be found in The Merck Manual of Diagnosis and Therapy, 20th Edition, published by Merck Sharp & Dohme Corp., 2018 (ISBN 0911910190, 978-0911910421); Robert S. Porter et al. (eds.), The Encyclopedia of Molecular Cell Biology and Molecular Medicine, published by Blackwell Science Ltd., 1999-2012 (ISBN 9783527600908); and Robert A. Meyers (ed.), Molecular Biology and Biotechnology: a Comprehensive Desk Reference, published by VCH Publishers, Inc., 1995 (ISBN 1-56081-569-8); Immunology by Werner Luttmann, published by Elsevier, 2006; Janeway’s Immunobiology, Kenneth Murphy, Allan Mowat, Casey Weaver (eds.), W. W. Norton & Company, 2016 (ISBN 0815345054, 978-0815345053); Lewin’s Genes XI, published by Jones & Bartlett Publishers, 2014 (ISBN-1449659055); Michael Richard Green and Joseph Sambrook, Molecular Cloning: A Laboratory Manual, 4th ed., Cold Spring Harbor Laboratory Press, Cold Spring Harbor, N.Y., USA (2012) (ISBN 1936113414); Davis et al., Basic Methods in Molecular Biology, Elsevier Science Publishing, Inc., New York, USA (2012) (ISBN 044460149X); Laboratory Methods in Enzymology: DNA, Jon Lorsch (ed.) Elsevier, 2013 (ISBN 0124199542); Current Protocols in Molecular Biology (CPMB), Frederick M. Ausubel (ed.), John Wiley and Sons, 2014 (ISBN 047150338X, 9780471503385), Current Protocols in Protein Science (CPPS), John E. Coligan (ed.), John Wiley and Sons, Inc., 2005; and Current Protocols in Immunology (CPI) (John E. Coligan, ADA M Kruisbeek, David H Margulies, Ethan M Shevach, Warren Strobe, (eds.) John Wiley and Sons, Inc., 2003 (ISBN 0471142735, 9780471142737), the contents of which are all incorporated by reference herein in their entireties.

In some embodiments of any of the aspects, the disclosure described herein does not concern a process for cloning human beings, processes for modifying the germ line genetic identity of human beings, uses of human embryos for industrial or commercial purposes or processes for modifying the genetic identity of animals which are likely to cause them suffering without any substantial medical benefit to man or animal, and also animals resulting from such processes.

Other terms are defined herein within the description of the various aspects of the invention.

All patents and other publications; including literature references, issued patents, published patent applications, and co-pending patent applications; cited throughout this application are expressly incorporated herein by reference for the purpose of describing and disclosing, for example, the methodologies described in such publications that might be used in connection with the technology described herein. These publications are provided solely for their disclosure prior to the filing date of the present application. Nothing in this regard should be construed as an admission that the inventors are not entitled to antedate such disclosure by virtue of prior invention or for any other reason. All statements as to the date or representation as to the contents of these documents is based on the information available to the applicants and does not constitute any admission as to the correctness of the dates or contents of these documents.

The description of embodiments of the disclosure is not intended to be exhaustive or to limit the disclosure to the precise form disclosed. While specific embodiments of, and examples for, the disclosure are described herein for illustrative purposes, various equivalent modifications are possible within the scope of the disclosure, as those skilled in the relevant art will recognize. For example, while method steps or functions are presented in a given order, alternative embodiments may perform functions in a different order, or functions may be performed substantially concurrently. The teachings of the disclosure provided herein can be applied to other procedures or methods as appropriate. The various embodiments described herein can be combined to provide further embodiments. Aspects of the disclosure can be modified, if necessary, to employ the compositions, functions and concepts of the above references and application to provide yet further embodiments of the disclosure. Moreover, due to biological functional equivalency considerations, some changes can be made in protein structure without affecting the biological or chemical action in kind or amount. These and other changes can be made to the disclosure in light of the detailed description. All such modifications are intended to be included within the scope of the appended claims.

In some embodiments, the present technology may be defined in any of the following numbered paragraphs:

1. A recombinant nucleic acid molecule, comprising:
a) a first segment encoding a recombinant Group 1 Dicistroviridae internal ribosome entry site (IRES) that has been modified to incorporate exogenous nucleotide sequences at a first site and a second site, and
b) a second segment encoding a protein, downstream from and operably linked to the first segment such that translation of the protein is repressed when the IRES is in an inactivated state;
wherein the first site comprises a first nucleotide sequence, and the second site comprises a second nucleotide sequence which is the reverse complement of at least a portion of the first nucleotide sequence.

2. The recombinant nucleic acid molecule of paragraph 1, wherein the nucleic acid molecule is an mRNA.

3. The recombinant nucleic acid molecule of paragraph 1, wherein the second nucleotide sequence is the reverse complement of substantially all of the first nucleotide sequence.

4. The recombinant nucleic acid molecule of paragraph 1, wherein the Group 1 Dicistroviridae IRES is a cricket paralysis virus (CrPV), a Kashmir bee virus (KBV), an acute bee paralysis virus (ABPV), or a Plauta Stali Intestine Virus (PSIV) IRES.

5. The recombinant nucleic acid molecule of paragraph 1, wherein the first and second sites are each independently selected from any of Site 1, Site 2, Site 3, Site 4, Site 5, Site 6, Site 7, and Site 8.

6. The recombinant nucleic acid molecule of paragraph 4, wherein the first and second sites respectively comprise: Site 1 and Site 2, Site 1 and Site 4, Site 1 and Site 5, Site 1 and Site 6, Site 1 and Site 7, Site 1 and Site 8, Site 2 and Site 6, Site 2 and Site 7, Site 4 and Site 6, Site 5 and Site 6, Site 5 and Site 7, Site 6 and Site 7, Site 8 and Site 2, Site 8 and Site 6, or Site 8 and Site 7.

7. The recombinant nucleic acid molecule of paragraph 1, wherein the first nucleotide sequence is 25-80 nt in length.

8. The recombinant nucleic acid molecule of paragraph 1, wherein the second nucleotide sequence is 8-25 nt in length.

9. The recombinant nucleic acid molecule of paragraph 1, wherein the first and second nucleotide sequences are capable of hybridizing when expressed in a eukaryotic cell under in vivo or in vitro conditions, causing the Group 1 Dicistroviridae IRES to fold into an inactivated state, wherein the eukaryotic cell is not a plant cell.

10. The recombinant nucleic acid molecule of paragraph 1, wherein the Group 1 Dicistroviridae IRES is configured to fold into an activated state in the presence of a trigger RNA molecule comprising a third nucleotide sequence, wherein the third nucleotide sequence is the reverse compliment of the first nucleotide sequence of the recombinant nucleic acid molecule of paragraph 1.

11. The recombinant nucleic acid molecule of paragraph 10, wherein the first nucleotide sequence is capable of hybridizing to the third nucleotide sequence when expressed in a eukaryotic cell under in vivo conditions, causing the Group 1 Dicistroviridae IRES to fold into the activated state, wherein the eukaryotic cell is not a plant cell.

12. A plasmid encoding the recombinant nucleic acid molecule of any one of the preceding paragraphs.

13. A eukaryotic cell comprising DNA encoding the recombinant nucleic acid molecule of any one of the preceding paragraphs, wherein the eukaryotic cell is not a plant cell and the DNA is:
a) integrated into the genomic DNA of the eukaryotic cell, or
b) present on a plasmid or viral vector present within the eukaryotic cell.

14. The eukaryotic cell of paragraph 13, wherein the cell is a) an animal cell, b) a human cell, or )c a primate cell.

16. A system for the control of gene expression, comprising:
a) the recombinant nucleic acid molecule of any of the preceding paragraphs; and
b) a trigger RNA molecule comprising a third nucleotide sequence, wherein the third nucleotide sequence is the reverse compliment of the first nucleotide sequence of the recombinant nucleic acid molecule.

17. A kit, comprising:
a) the plasmid of paragraph 12; and
b) a trigger RNA molecule comprising a third nucleotide sequence, wherein the third nucleotide sequence is the reverse compliment of the first nucleotide sequence of the recombinant nucleic acid molecule.

18. A recombinant mRNA molecule, comprising:
a) a first segment encoding a first protein,
b) a second segment, downstream of the first segment, encoding a recombinant Group 1 Dicistroviridae internal ribosome entry site (IRES) that has been modified to incorporate exogenous nucleotide sequences at a first site and a second site, and
c) a third segment encoding a second protein, downstream from and operably linked to the second segment such that translation of the second protein is repressed when the IRES is in an inactivated state;
wherein transcription of the recombinant mRNA molecule is dependent on a polymerase, and wherein the first site comprises a first nucleotide sequence, and the second site comprises a second nucleotide sequence which is the reverse complement of at least a portion of the first nucleotide sequence.

19. The recombinant mRNA molecule of paragraph 18, wherein transcription of the recombinant mRNA molecule is dependent on:
a) an RNA polymerase II;
b) a polymerase other than RNA polymerase II;
c) a T7 polymerase; and/or
d) an SP6 polymerase.

20. A method of activating and/or modulating expression of a protein, comprising:
a) providing a eukaryotic cell engineered to express the recombinant nucleic acid molecule of any of the preceding paragraphs; and
b) introducing a trigger RNA molecule comprising a third nucleotide sequence into the eukaryotic cell, wherein the third nucleotide sequence is the reverse compliment of the first nucleotide sequence of the recombinant nucleic acid molecule;
wherein the first nucleotide sequence hybridizes to the third nucleotide sequence under in vivo conditions, causing the Group 1 Dicistroviridae IRES to fold into an activated state, further wherein the eukaryotic cell is not a plant cell.

21. The method of paragraph 20, wherein the eukaryotic cell engineered to express the recombinant nucleic acid molecule is provided by introducing the recombinant nucleic acid molecule of any of the preceding paragraphs into the eukaryotic cell.

23. A method for detecting viral infection of a eukaryotic cell, comprising:
a) providing a eukaryotic cell engineered to express the recombinant nucleic acid molecule of any of the preceding paragraphs, wherein the first nucleotide sequence of the recombinant nucleic acid molecule is configured to be the reverse compliment of at least a portion of a mRNA sequence unique to a virus; and
b) determining whether the eukaryotic cell is infected with the virus by detecting and/or measuring the presence of the protein encoded by the second segment of the recombinant nucleic acid molecule, wherein the eukaryotic cell is not a plant cell.

24. The method of paragraph 23, wherein the virus is a Dengue virus or a Zika virus.

25. A method for controlling differentiation of a eukaryotic cell, comprising
a) providing a eukaryotic cell engineered to express the recombinant nucleic acid molecule of any of the preceding paragraphs; and
b)culturing the eukaryotic cell;
wherein the first nucleotide sequence of the recombinant nucleic acid molecule is configured to be the reverse compliment of at least a portion of a mRNA sequence unique to a selected cell type, and the protein encoded by the second segment of the recombinant nucleic acid molecule comprises a toxin or a protein that causes apoptosis of the selected cell type, further wherein the eukaryotic cell is not a plant cell.

27. A vector, comprising: a) DNA encoding an mRNA which comprises a recombinant nucleic acid molecule, wherein the recombinant nucleic acid molecule comprises:
i) a first segment encoding a recombinant Group 1 Dicistroviridae internal ribosome entry site (IRES) that has been modified to incorporate exogenous nucleotide sequences at a first site and a second site, and
ii) a second segment encoding a protein, downstream from and operably linked to the first segment such that translation of the protein is repressed when the IRES is in an inactivated state;
wherein the first site comprises a first nucleotide sequence, and the second site comprises a second nucleotide sequence which is the reverse complement of at least a portion of the first nucleotide sequence; and
wherein the Group 1 Dicistroviridae IRES is configured to activate expression of the protein in response to the presence of an mRNA which comprises a segment that is the reverse complement of the first nucleotide sequence.

28. A prokaryotic cell comprising DNA encoding the recombinant nucleic acid molecule of any one of the preceding paragraphs, wherein the DNA is:
a) integrated into the genomic DNA of the prokaryotic cell, or
b) present on a plasmid or viral vector present within the prokaryotic cell.

In some embodiments, the present technology may be defined in any of the following numbered paragraphs:

1. A recombinant nucleic acid molecule, comprising:
a) a first segment encoding a recombinant Group 1 Dicistroviridae internal ribosome entry site (IRES) that has been modified to incorporate exogenous nucleotide sequences at a first site and a second site, and
b) a second segment encoding a protein, downstream from and operably linked to the first segment such that translation of the protein is repressed when the IRES is in an inactivated state;
wherein the first site comprises a first nucleotide sequence, and the second site comprises a second nucleotide sequence which is the reverse complement of at least a portion of the first nucleotide sequence.

2. A recombinant nucleic acid molecule, comprising from 5′ to 3′:
a) a first segment encoding a recombinant viral internal ribosome entry site (IRES) that has been modified at a first site to incorporate a first exogenous nucleotide sequence and modified at a second site to incorporate a second exogenous nucleotide sequence; and
b) a second segment encoding a protein, downstream from and operably linked to the first segment such that translation of the protein is repressed when the IRES is in an inactivated state;
wherein the second nucleotide sequence is the reverse complement of at least a portion of the first nucleotide sequence.

3. The recombinant nucleic acid molecule of paragraph 2, wherein the IRES that is modified is a Group 1 Discistroviridae IRES; a Hepacivirus IRES; or an Enterovirus IRES.

4. The recombinant nucleic acid molecule of any of paragraphs 1-3, wherein the IRES that is modified is an IRES from a mammalian pathogenic virus or mammalian commensal virus.

5. The recombinant nucleic acid molecule of paragraph 4, wherein the IRES that is modified is an IRES from a human pathogenic virus or human commensal virus.

6. The recombinant nucleic acid molecule of any of paragraphs 1-5, wherein the nucleic acid molecule is an mRNA.

7. The recombinant nucleic acid molecule of any of paragraphs 1-6, wherein the second nucleotide sequence is the reverse complement of substantially all of the first nucleotide sequence.

8. The recombinant nucleic acid molecule of any of paragraphs 1-7, wherein the Group 1 Dicistroviridae IRES is a cricket paralysis virus (CrPV), a Kashmir bee virus (KBV), an acute bee paralysis virus (ABPV), a Plauta Stali Intestine Virus (PSIV) IRES; an aphid lethal paralysis virus (ALPV) IRES; a black queen cell virus (BQCV) IRES; a Drosophila C virus (DCV) IRES; a Himetobi P virus (HiPV) IRES; a Homalodisca coagulata virus-1 (HoCV-1) IRES; a Rhopalosiphum padi virus (RhPV) IRES; and a Triatoma virus (TrV) IRES .

9. The recombinant nucleic acid molecule of any of paragraphs 2-7, wherein the Hepacivirus IRES is a hepatitis c virus (HCV) IRES.

10. The recombinant nucleic acid molecule of any of paragraphs 2-7, wherein the Enterovirus IRES is a poliovirus (PV) IRES or enterovirus 71 (EV71) IRES.

11. The recombinant nucleic acid molecule of any of paragraphs 1-10, wherein the first and second sites are each independently selected from any of Site 1, Site 2, Site 3, Site 4, Site 5, Site 6, Site 7, and Site 8.

12. The recombinant nucleic acid molecule of paragraph 11, wherein the first and second sites respectively comprise: Site 1 and Site 2, Site 1 and Site 4, Site 1 and Site 5, Site 1 and Site 6, Site 1 and Site 7, Site 1 and Site 8, Site 2 and Site 6, Site 2 and Site 7, Site 4 and Site 6, Site 5 and Site 6, Site 5 and Site 7, Site 6 and Site 7, Site 8 and Site 2, Site 8 and Site 6, or Site 8 and Site 7.

13. The recombinant nucleic acid molecule of any of paragraphs 11-12, wherein the first and second sites respectively comprise: Site 1 and Site 2, Site 1 and Site 8, Site 2 and Site 7, Site 6 and Site 7, or Site 8 and Site 6.

14. The recombinant nucleic acid molecule of any of paragraphs 11-13, wherein the first and second sites respectively comprise: Site 6 and Site 7, or Site 8 and Site 6.

15. The recombinant nucleic acid molecule of any of paragraphs 1-14, wherein the first nucleotide sequence is 25-80 nt in length.

16. The recombinant nucleic acid molecule of paragraph 15, wherein the first nucleotide sequence is 40-50 nt in length.

17. The recombinant nucleic acid molecule of paragraph 15, wherein the second nucleotide sequence is 8-25 nt in length.

18. The recombinant nucleic acid molecule of any of paragraphs 1-17, wherein the second nucleotide sequence is 6-15 nt in length.

19. The recombinant nucleic acid molecule of any of paragraphs 1-18, wherein the first nucleotide sequence is from 2.5x to 8x longer than the second nucleotide sequence.

20. The recombinant nucleic acid molecule of any of paragraphs 1-19, wherein the first nucleotide sequence is the reverse complement of a sequence found in a target eukaryotic organism, target prokaryotic organism, or target virus.

21. The recombinant nucleic acid molecule of paragraph 20, wherein the target prokaryotic organism or target virus is a human pathogen.

22. The recombinant nucleic acid molecule of paragraph 21, wherein the target virus is Zika virus or a coronavirus.

23. The recombinant nucleic acid molecule of paragraph 22, wherein the coronavirus is SARS-CoV-2.

24. The recombinant nucleic acid molecule of any of paragraphs 1-23, wherein the protein produces a detectable signal.

25. The recombinant nucleic acid molecule of any of paragraphs 1-24, wherein the first and second nucleotide sequences are capable of hybridizing when expressed in a eukaryotic cell under in vivo or in vitro conditions, causing the IRES to fold into an inactivated state, wherein the eukaryotic cell is not a plant cell.

26. The recombinant nucleic acid molecule of any of paragraphs 1-25, wherein the IRES is configured to fold into an activated state in the presence of a trigger RNA molecule comprising a third nucleotide sequence, wherein the third nucleotide sequence is the reverse compliment of the first nucleotide sequence of the recombinant nucleic acid molecule of paragraph 1.

27. The recombinant nucleic acid molecule of paragraph 26, wherein the first nucleotide sequence is capable of hybridizing to the third nucleotide sequence when expressed in a eukaryotic cell under in vivo conditions, causing the IRES to fold into the activated state, wherein the eukaryotic cell is not a plant cell.

28. An expression construct comprising a sequence encoding or comprising the recombinant nucleic acid molecule of any of paragraphs 1-27, and further comprising, 5′ and/or 3′ of the sequence encoding or comprising the recombinant nucleic acid molecule of any of paragraphs 1-27, one or more of:
a) an IRES pseudoknot sequence;
b) an IRES pseudoknot sequence found in the wild-type sequence of a virus in which the IRES naturally occurs;
c) a promoter and/or upstream activating factor binding sequence;
d) a stop codon;
e) a stem-loop;
f) a 5′ cap;
g) a reporter gene; and
h) a poly-A tail.

29. The expression construct of paragraph 28, wherein the expression construct comprises, 5′ of the sequence encoding or comprising the recombinant nucleic acid molecule of any of paragraphs 1-27, one or more of:
a) an IRES pseudoknot sequence;
b) an IRES pseudoknot sequence found in the wild-type sequence of a virus in which the IRES naturally occurs;
c) a promoter and/or upstream activating factor binding sequence;
d) a stop codon;
e) a stem-loop;
f) a 5′ cap; and
g) a reporter gene.

30. The expression construct of any of paragraphs 28-29, wherein:
a) the promoter is selected from a SP6,T3, araBAD, trp, lac, Ptac, and pL promoters; and/or
b) the upstream activating factor binding sequence is upstream activation factor binding DNA sequence (UAF2) from Saccharomyces cerevisiae.

31. The expression construct or recombinant nucleic acid sequence of any of paragraphs 1-30, wherein transcription of the recombinant nucleic acid molecule is dependent on:
a) an RNA polymerase II;
b) a polymerase other than RNA polymerase II;
c) a T7 polymerase; and/or
d) an SP6 polymerase.

32. A recombinant mRNA molecule, comprising:
a) a first segment encoding a first protein,
b) a second segment, downstream of the first segment, encoding a recombinant Group 1 Dicistroviridae internal ribosome entry site (IRES) that has been modified to incorporate exogenous nucleotide sequences at a first site and a second site, and
c) a third segment encoding a second protein, downstream from and operably linked to the second segment such that translation of the second protein is repressed when the IRES is in an inactivated state;
wherein transcription of the recombinant mRNA molecule is dependent on a polymerase, and wherein the first site comprises a first nucleotide sequence, and the second site comprises a second nucleotide sequence which is the reverse complement of at least a portion of the first nucleotide sequence.

33. The recombinant mRNA molecule of paragraph 32, wherein transcription of the recombinant mRNA molecule is dependent on:
a) an RNA polymerase II;
b) a polymerase other than RNA polymerase II;
c) a T7 polymerase; and/or
d) an SP6 polymerase.

34. A plasmid encoding the recombinant nucleic acid molecule, expression construct, or recombinant mRNA molecule of any one of the preceding paragraphs.

35. A eukaryotic cell comprising DNA encoding the recombinant nucleic acid molecule, expression construct, or recombinant mRNA molecule of any one of the preceding paragraphs, wherein the eukaryotic cell is not a plant cell and the DNA is:
a) integrated into the genomic DNA of the eukaryotic cell, or
b) present on a plasmid or viral vector present within the eukaryotic cell.

36. The eukaryotic cell of paragraph 35, wherein the cell is a) an animal cell, b) a human cell, or )c a primate cell.

37. A system for the control of gene expression, comprising:
a) the recombinant nucleic acid molecule of any of the preceding paragraphs; and
b) a trigger RNA molecule comprising a third nucleotide sequence, wherein the third nucleotide sequence is the reverse compliment of the first nucleotide sequence of the recombinant nucleic acid molecule.

38. A kit, comprising:
a) the plasmid of paragraph 34; and
b) a trigger RNA molecule comprising a third nucleotide sequence, wherein the third nucleotide sequence is the reverse compliment of the first nucleotide sequence of the recombinant nucleic acid molecule.

39. A method of activating and/or modulating expression of a protein, comprising:
a) providing a eukaryotic cell engineered to express the recombinant nucleic acid molecule of any of the preceding paragraphs; and
b) introducing a trigger RNA molecule comprising a third nucleotide sequence into the eukaryotic cell, wherein the third nucleotide sequence is the reverse compliment of the first nucleotide sequence of the recombinant nucleic acid molecule;
wherein the first nucleotide sequence hybridizes to the third nucleotide sequence under in vivo conditions, causing the IRES to fold into an activated state, further wherein the eukaryotic cell is not a plant cell.

40. The method of paragraph 39, wherein the eukaryotic cell engineered to express the recombinant nucleic acid molecule is provided by introducing the recombinant nucleic acid molecule of any of the preceding paragraphs into the eukaryotic cell.

41. A method for detecting viral infection of a eukaryotic cell, comprising:
a) providing a eukaryotic cell engineered to express the recombinant nucleic acid molecule of any of the preceding paragraphs, wherein the first nucleotide sequence of the recombinant nucleic acid molecule is configured to be the reverse compliment of at least a portion of a mRNA sequence unique to a virus; and
b)determining whether the eukaryotic cell is infected with the virus by detecting and/or measuring the presence of the protein encoded by the second segment of the recombinant nucleic acid molecule, wherein the eukaryotic cell is not a plant cell.

42. The method of paragraph 41, wherein the virus is a Dengue virus or a Zika virus.

43. A method for controlling differentiation of a eukaryotic cell, comprising
a) providing a eukaryotic cell engineered to express the recombinant nucleic acid molecule of any of the preceding paragraphs; and
b)culturing the eukaryotic cell;
wherein the first nucleotide sequence of the recombinant nucleic acid molecule is configured to be the reverse compliment of at least a portion of a mRNA sequence unique to a selected cell type, and the protein encoded by the second segment of the recombinant nucleic acid molecule comprises a toxin or a protein that causes apoptosis of the selected cell type, further wherein the eukaryotic cell is not a plant cell.

44. A vector, comprising: a sequence encoding a recombinant nucleic acid molecule of any of the preceding paragraphs; wherein the modified IRES is configured to activate expression of the protein in response to the presence of an mRNA which comprises a segment that is the reverse complement of the first nucleotide sequence.

45. A prokaryotic cell comprising DNA encoding the recombinant nucleic acid molecule of any one of the preceding paragraphs, wherein the DNA is:
a) integrated into the genomic DNA of the prokaryotic cell; or
b) present on a plasmid or viral vector present within the prokaryotic cell.

Specific elements of any of the foregoing embodiments can be combined or substituted for elements in other embodiments. Furthermore, while advantages associated with certain embodiments of the disclosure have been described in the context of these embodiments, other embodiments may also exhibit such advantages, and not all embodiments need necessarily exhibit such advantages to fall within the scope of the disclosure.

The technology described herein is further illustrated by the following examples which in no way should be construed as being further limiting.

### EXAMPLES

The following non-limiting examples are provided to further illustrate the present disclosure.

### Example 1: Screening for Recombinant IRES Riboswitches

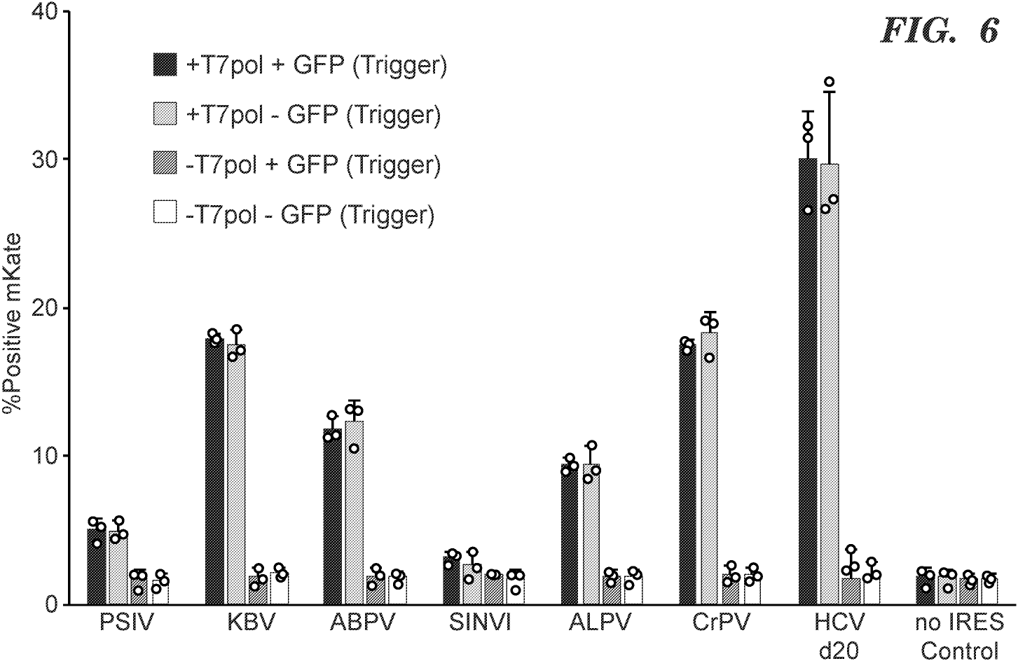

The process of designing recombinant IRES riboswitches began with the selection of IRES modules from viral databases and testing them in a human embryonic kidney 293 (HEK293) cell-based transfection assay. Co-transfected with these constructs, the T7 polymerase was found not to 5′ cap mRNA, resulting in a dramatic enhancement of mKate expression by the IRES modules, as illustrated by FIG. 6. The IRES of Hepatitis C virus (HCVd20) was used as a control for this assay, but was not pursued due to its extensive structural differences compared to the rest of the selected IRES modules and reported reliance of activity on small RNAs. Based on this initial screen, the cricket paralysis virus (CrPV), kashmir bee virus (KBV), and acute bee paralysis virus (ABPV) IRES modules were selected for further study, with focus on the CrPV IRES due to the abundance of existing structural information.

In order to test whether disruption of the structure of these IRES modules could reduce their translational initiation capability, variants of the CrPV, KBV and ABVP IRES modules, which incorporated exogenous nucleotide sequences, were engineered. It was theorized that the insertion of two reverse complement segments of DNA into two sites of the IRES module could be used to distort the functional configuration/structure of the IRES module (e.g., due to hybridization of these two segments). The two reverse complement segments of DNA were further designed to be released by a trigger RNA sequence (trRNA) that is the reverse complement of one of the segments and which includes a longer overlapping portion compared to the overlap between the first and second segments. Specifically, the longer segment of inserted DNA (40-50 base pairs) was the reverse complement of a portion of the target trigger and the shorter segment (10-15 base pairs) was the reverse complement of a portion of the first segment. Eight sites were selected where insertions would not individually break IRES module activity (i.e. Sites 1-8 shown in FIG. 3) and avoided Loop 3, whose complete functionality is crucial for any level of IRES module activity.

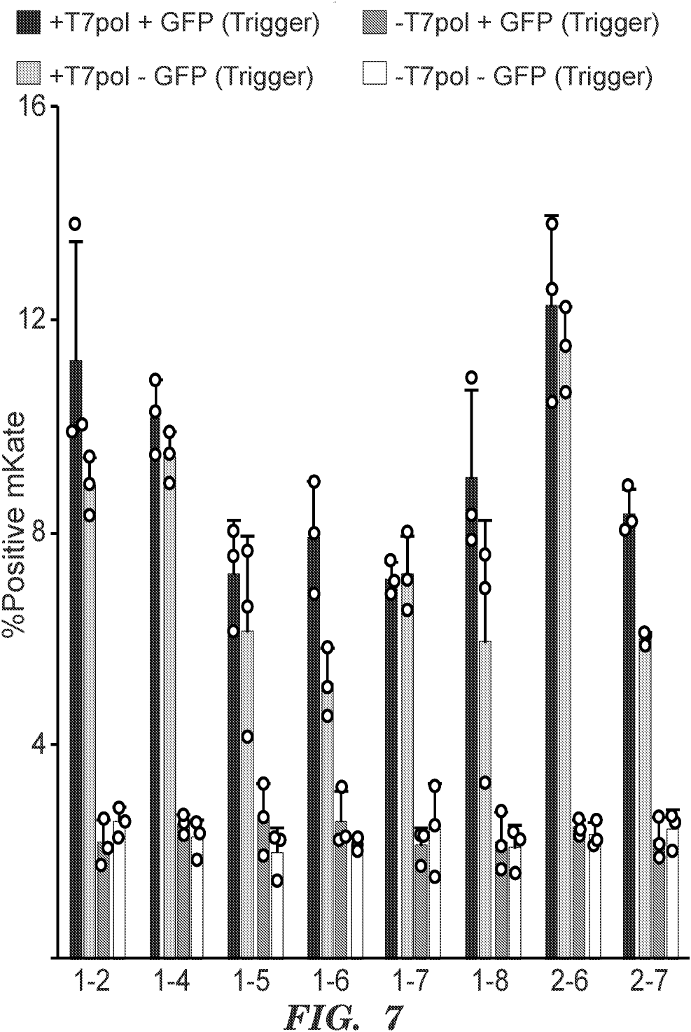

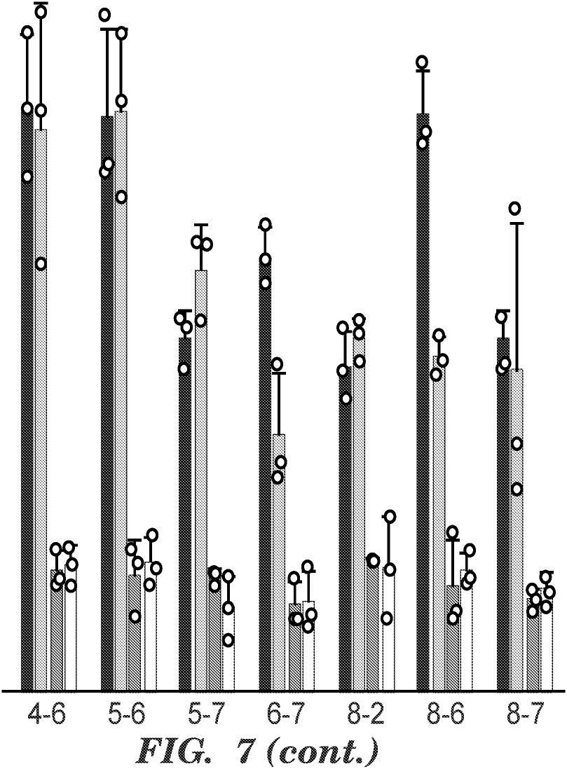

A set of 15 fold combinations were tested using the same assay for IRES activity determination (FIG. 7). Each fold combination was named using the format (long segment site number - short segment site number). In this initial study, GFP mRNA was used as a trigger RNA. Although folds 1-2, 1-8, and 2-7 led to reproducible fold changes in this initial screen, further testing showed a lack of transferability of the same folds towards other target triggers. It was decided to continue engineering folds 6-7 and 8-6, which both showed 1.7x fold increases in percentage of mKate positive cells when the trigger RNA was transfected concurrently.

### Example 2: Optimization of Recombinant IRES Riboswitches

Practical applications of the presently disclosed recombinant IRES riboswitches will often require high fold-changes of downstream protein translation following trigger induction. Therefore, a reduction in the leakiness of the two hits from the initial screen (i.e., folds 6-7 and 8-6) was sought. Previous studies suggest that IRES pseudoknots are critical for ribosome recruitment. As such, a study was conducted to determine whether the distortion of an IRES module along with the breaking of the pseudoknot at the same locations (after insertion site 7 for fold 6-7 and after insertion site 6 for fold 8-6) would reduce the leakiness of the IRES module. New recombinant IRES riboswitches were designed such that the base pairs in insertion 1 following where insertion 2 would anneal were reverse complements of the base pairs corresponding to the pseudoknots. In the absence of a trigger, this annealing would prevent correct pseudoknot folding, i.e., creating a pseudoknot breaking site (PB site). Designing recombinant IRES riboswitches with the PB site led to a reduction of the “OFF” (no trigger) state to levels observed when no IRES module was present and increased the “ON” (with trigger) to “OFF” (without trigger) fold-change from 1.7x to 2.5x for fold 8-6, as shown by FIG. 8. Further optimization of the melting temperature of the annealing portion showed a dependence of IRES riboswitch behavior on this parameter but no improvements in the “ON” to “OFF” fold-change.

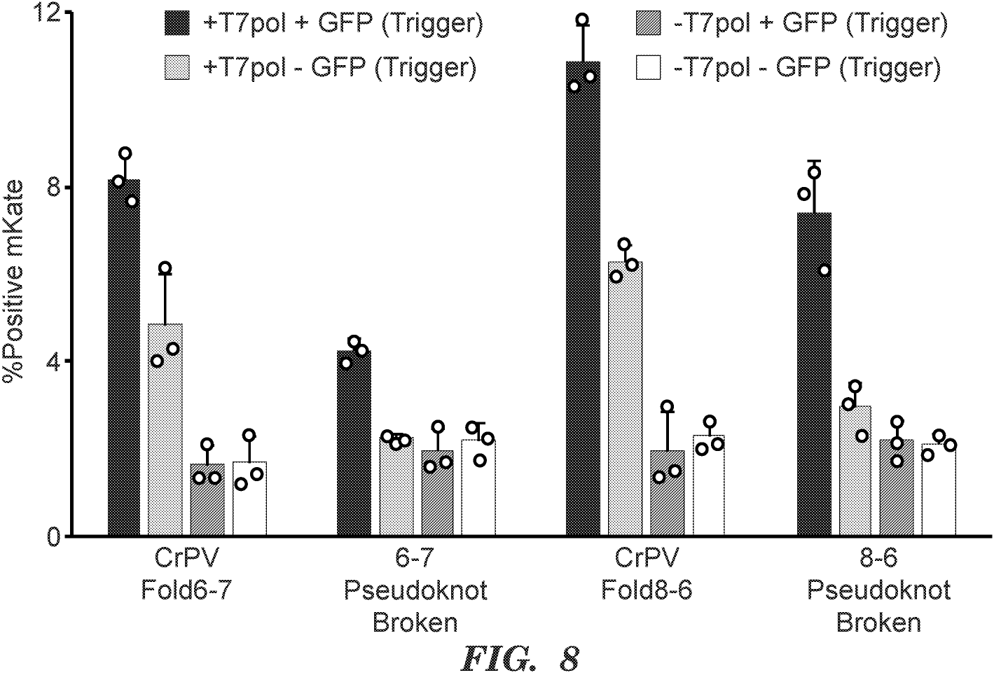

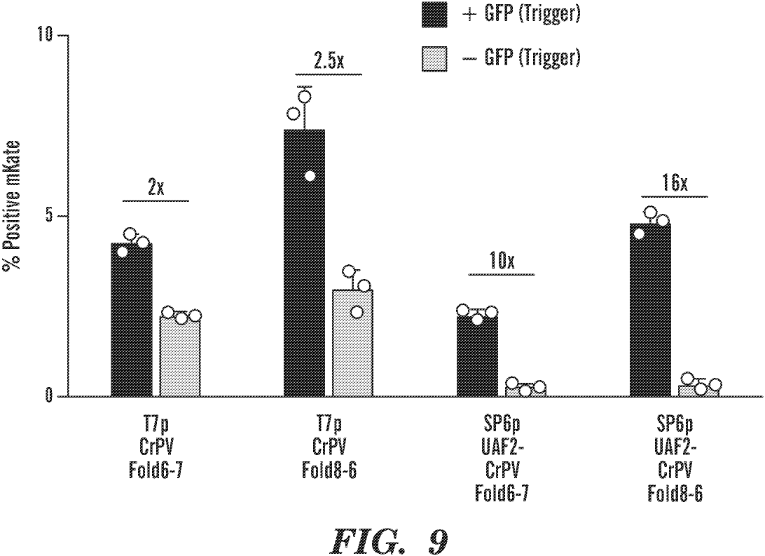

Based on these findings, it was theorized that the remaining leakiness was caused by non-specific binding of the T7 promoter sequence by endogenous RNA polymerase II, leading to 5′ capping and bypassing of IRES-mediated translational initiation. To find a suitable polymerase-promoter replacement pair, we placed sfGFP under different promoters with high RNA expression levels and transfected HEK293T cells (without corresponding polymerases). It was found that P_{SP6} was significantly less leaky than P_{T7} and its analogues. Upstream activation sequences were tested for RNA polymerase I, which have been shown to decrease RNA polymerase II binding. Our findings demonstrated that an upstream activation factor binding DNA sequence from Saccharomyces cerevisiae decreased leakiness (named UAF2). By combining these two methods/techniques/approaches, we minimized leakiness substantially and reached an “ON” to “OFF” trigger-mRNA based induction of 15.9x fold, as shown by FIG. 9. Similar folds of induction could be reached by adding stop codons and stem loops before the recombinant IRES riboswitch. Further increases in “ON” to “OFF” fold change could be achieved using a recombinant IRES riboswitch that hides a kozak sequence downstream of the riboswitch, but this method requires extensive screening (only 1 out of 9 kozak toeholds improved fold change).

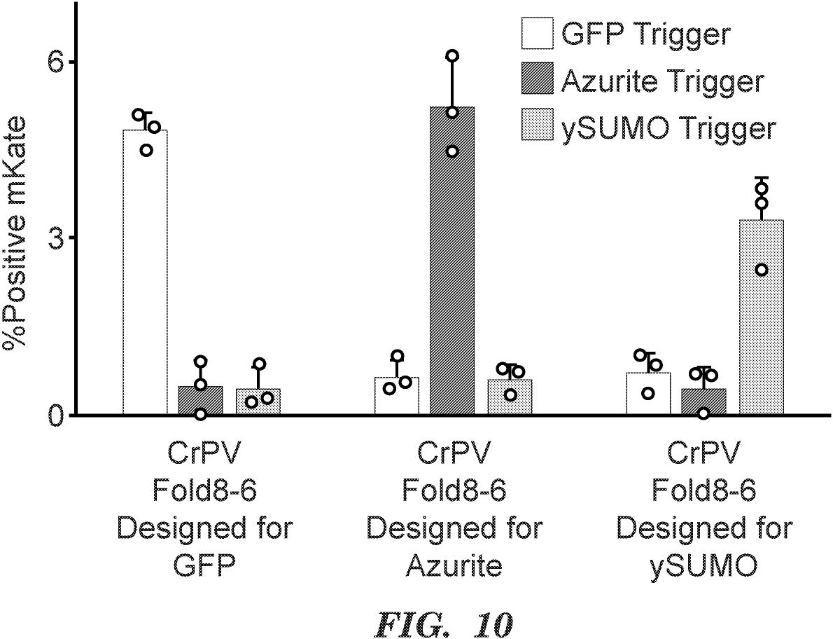

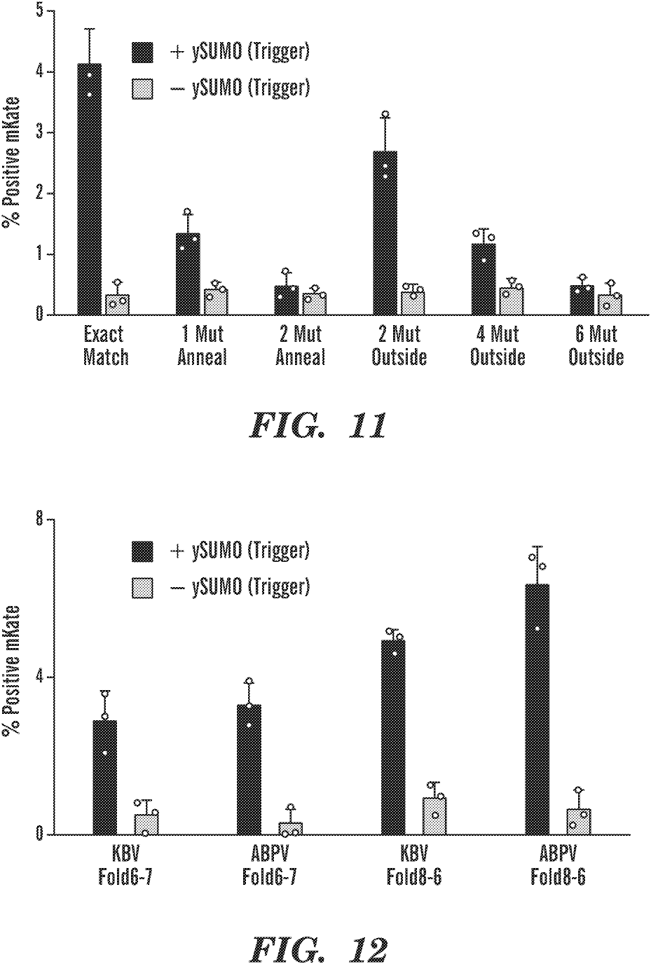

To further characterize the recombinant IRES riboswitch of the present disclosure, we tested the capability of these riboswitches to sense trigger mRNA from a number of sources. In particular, we designed folds to detect GFP, Azurite, and yeast SUMO mRNA. By exposing each of the folds to each of the three types of mRNA, we demonstrate that the recombinant IRES riboswitch are orthogonal and can be designed for most target mRNAs (FIG. 10). We further tested the sensitivity of our folds by mutating the insertions both inside insertion 1 and 2 (Mut Anneal) or in insertion 1 but not insertion 2 (Mut Outside). We found that recombinant IRES riboswitch are very sensitive to mutations within insertion 2, indicated by 3x fold decreases in activation level with a single mutation, but not as sensitive to mutations outside of insertion 2 (FIG. 11). This study therefor confirmed that recombinant IRES riboswitches can be designed for different mRNAs while maintaining specificity.

We then aimed to expand possible PB sites for easier recombinant IRES riboswitch design. Given the sequence differences and structural similarities between the IRES modules, we sought to achieve this by creating folds using different IRES modules. In creating recombinant IRES riboswitches using both KBV and ABPV IRES modules, we observed similar fold changes of trigger-induced translation to the CrPV IRES modules (FIG. 12). These alternative IRES eToeholds effectively expand the range of possible PB sites.

### EXAMPLE 3: Modular RNA-responsive Elements for Eukaryotic Translational Control

Robust and easily programmed RNA-responsive modules are highly desirable for a variety of applications in biotechnology. Simple RNA-responsive elements with translational control over transgenes remain unrealized in eukaryotes¹⁻³. Described herein are eukaryotic toehold switches (eToeholds) as modular riboregulators, based on internal ribosome entry site (IRES) sequences that provide translational control over transgenes in response to specific RNA sequences. It is demonstrated herein that eToeholds, designed through optimization of RNA annealing, sense and respond to the presence of trigger RNAs with up to 16-fold induction of transgenes in a range of eukaryotic cells. It is also demonstrated that eToeholds can discriminate between infection status, cell state, and cell type in mammalian cells, based on the presence of exogenous or endogenous RNA transcripts.

Synthetic biology techniques for sensing and responding to specific intracellular RNAs are desirable for therapeutic and diagnostic applications, as they provide a means to discriminate and target specific cells, tissues, and organisms, and can serve as building blocks for sophisticated genetic circuits. RNA-based prokaryotic modules, called toehold switches, were developed for detecting specific RNA transcripts^{1,4}. Toehold switches selectively repress translation of an in-cis reporter gene by sequestering the ribosome binding site (RBS) upstream of the reporter gene in a stem loop structure in the absence of a trigger RNA (trRNA). The RBS is released when a trRNA binds the toehold switch and opens the stem loop structure, thus initiating translation of the reporter gene. Eukaryotic translation, however, is far more complicated and is typically regulated by several factors, including 5′ modified capping recruited by RNA polymerase II, a poly-adenosine (polyA) tail for mRNA stabilization, as well as a Kozak consensus sequence for protein translational regulation. Although the Kozak sequence improves ribosomal binding, it is not as critical to translation as the prokaryotic RBS; previously developed Kozak-based toehold switches have only achieved up to two-fold trRNA-driven induction of eukaryotic translation⁵. The 5′ cap dominates translational regulation mechanisms and is a major challenge for any eukaryotic RNA-sensing riboswitch that functions at the level of translation.

More complex RNA-based switches have been developed, utilizing Cas9 expression and engineered folding of the guide RNA (gRNA) to hide sequences essential for function^{2,3}. Unfolding of the gRNA by trRNA leads to activation of the Cas9 enzyme and corresponding downstream regulation. However, these mechanisms induce only modest fold changes (in both eukaryotes and prokaryotes). An alternative technique involves the use of a ribozyme that cleaves the polyA tail upon small molecule induction^{6,7}. This approach has not yet been expanded for larger nucleotide sequences and leads to the degradation of the RNA. This makes the sequence triggered reaction irreversible and incapable of sensing temporal changes in RNA level. Although recent studies have utilized ribozymes^{8,9} to respond to short nucleotide oligomers, these ribozyme-based sensors are not yet compatible with the detection of longer trRNAs, including endogenous transcripts.

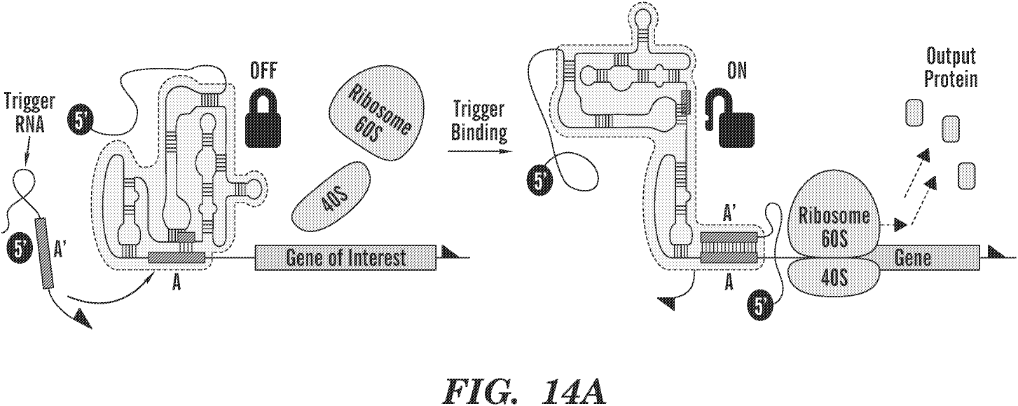

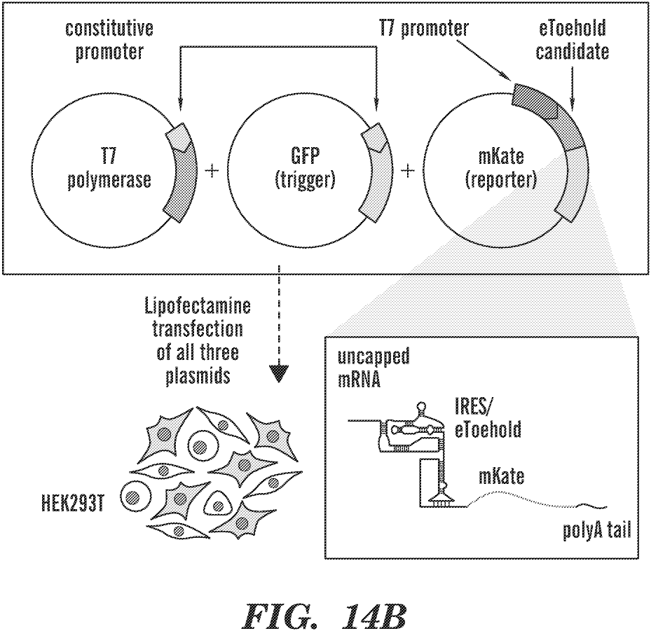

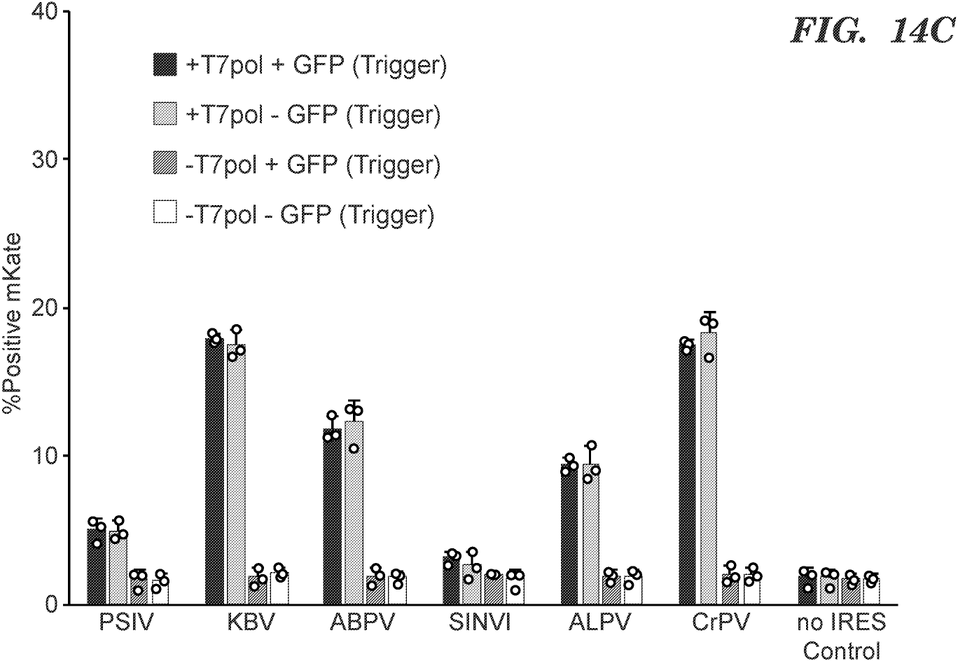

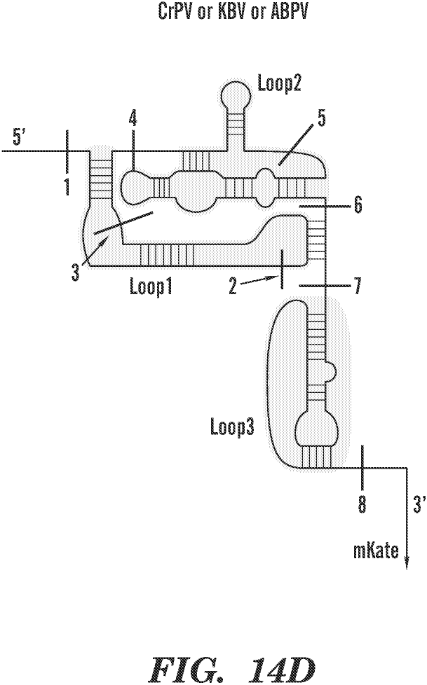

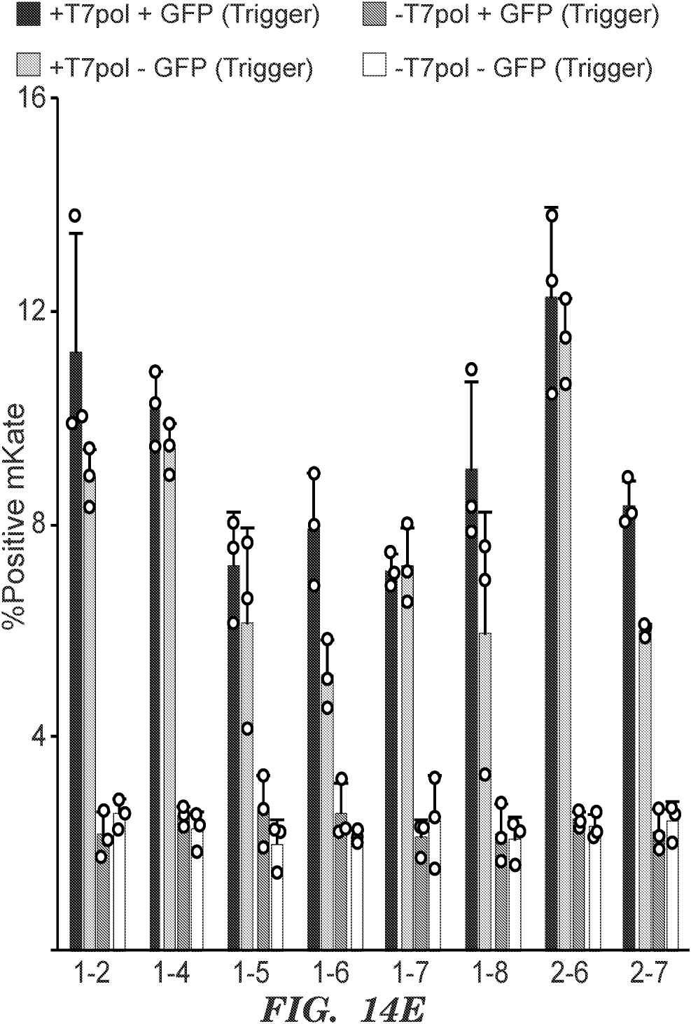

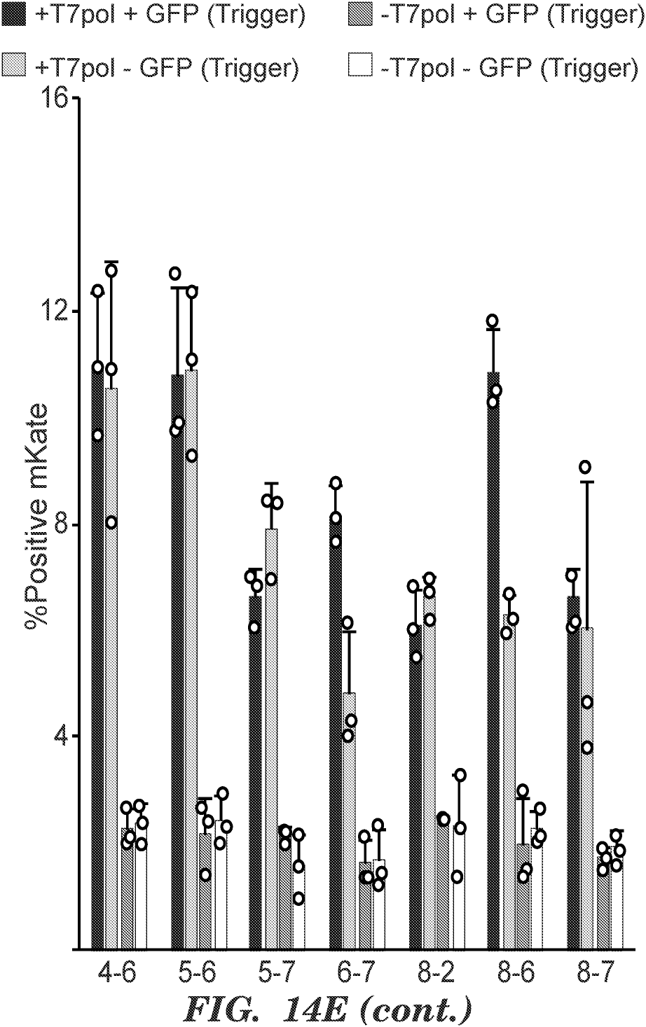

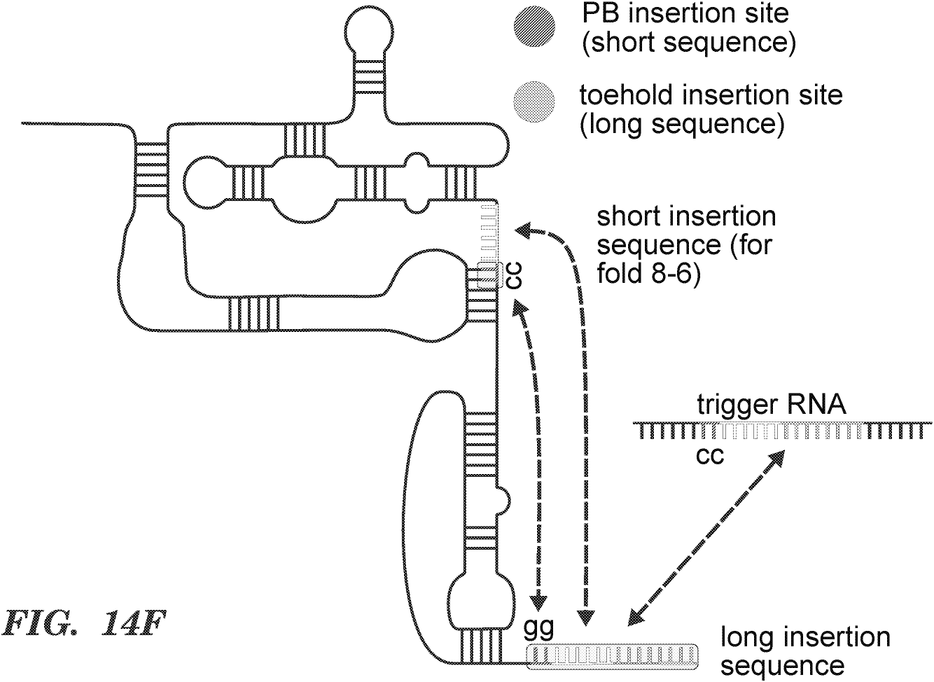

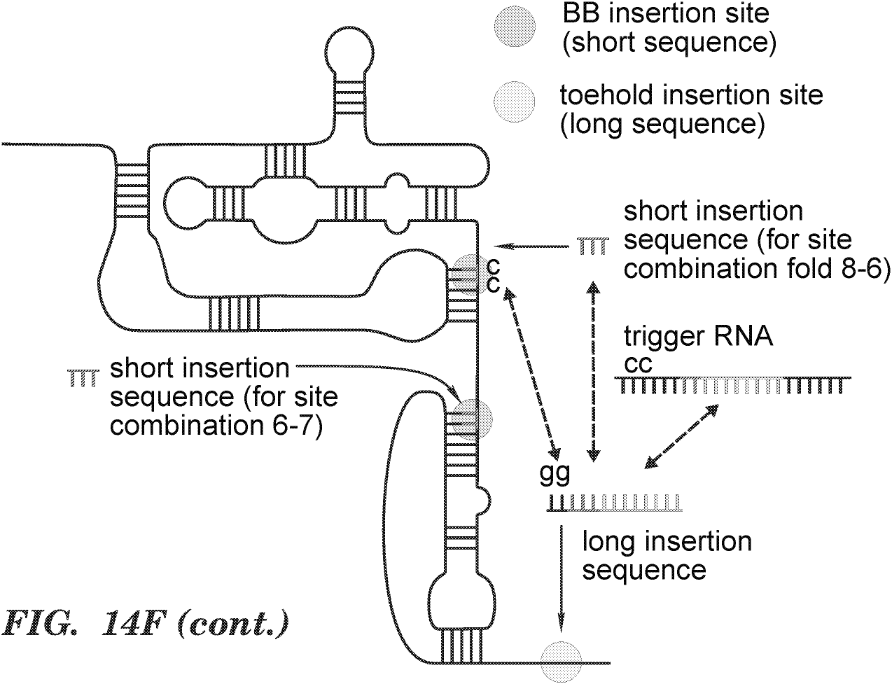

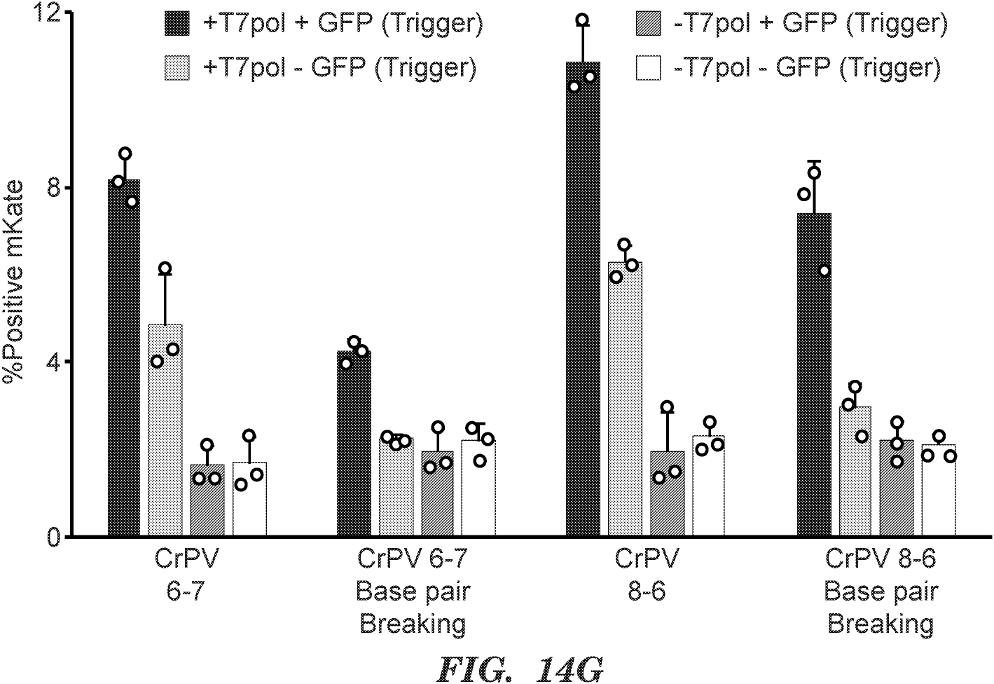

IRESs are endogenous and viral eukaryotic mRNA elements whose structure has evolved to initiate protein translation independent of mRNA 5′ capping and polyadenylation. Described herein is the development of RNA-based eukaryotic modules, called eToeholds, that permit the regulated translation of in-cis reporter genes by the presence of specific trRNAs. eToeholds incorporate modified IRESs that are designed to be inactive until sense-antisense interactions with a specific trRNA cause activation (FIG. 14A). Using this system, up to 16-fold trRNA-induced translation of transgenes is achieved. It is demonstrated that eToeholds have functionality in human and yeast cells, as well as mammalian cell-free lysates. It is further demonstrated that stable cell lines expressing eToeholds can be used to sense natural viral infection (by Zika virus) and viral transcripts (SARS-CoV-2 constructs). It is also demonstrated that eToeholds have the capability to discriminate different cell states and cell types by selectively activating protein translation based on endogenous RNA levels.

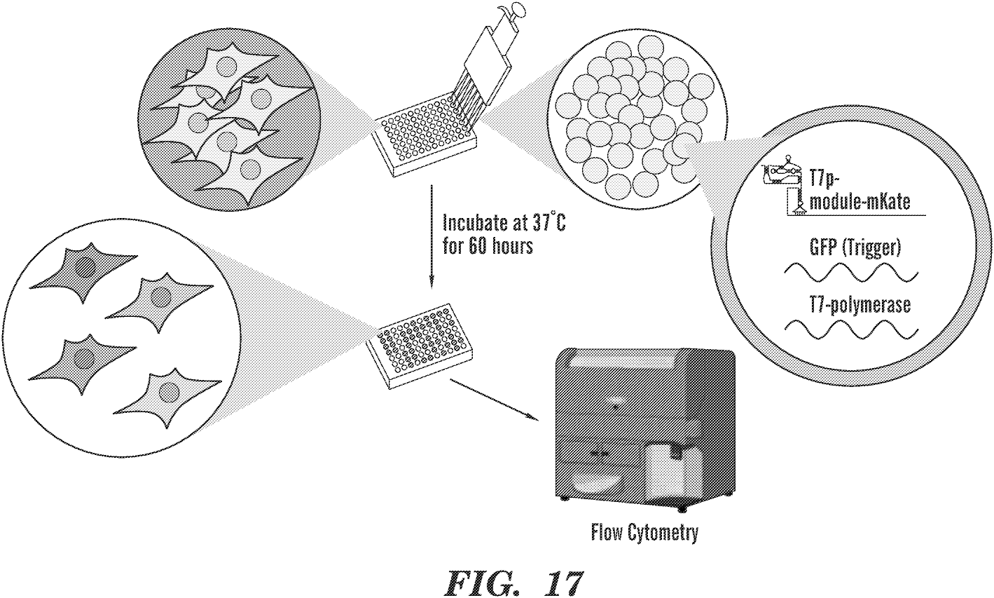

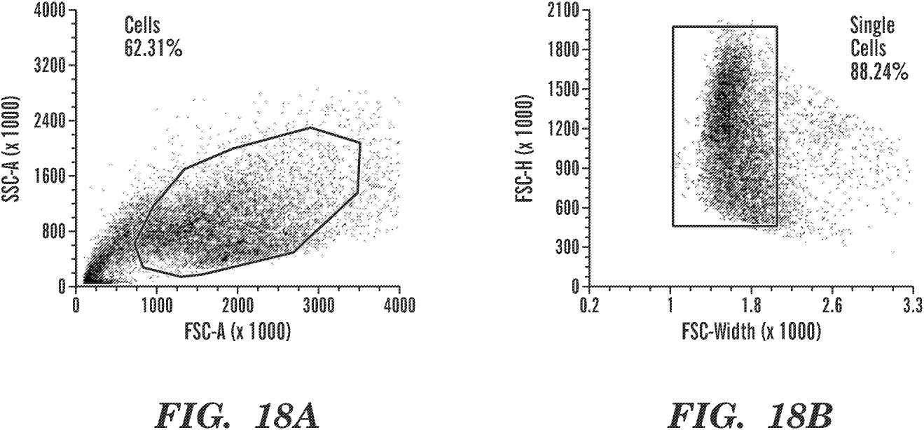

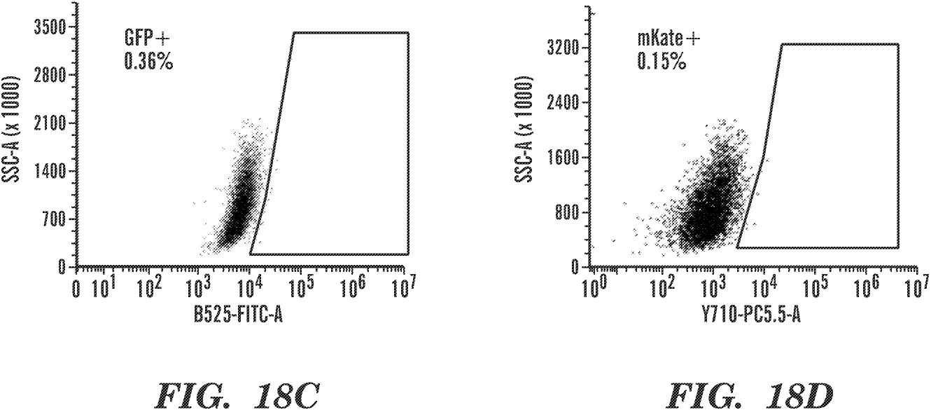

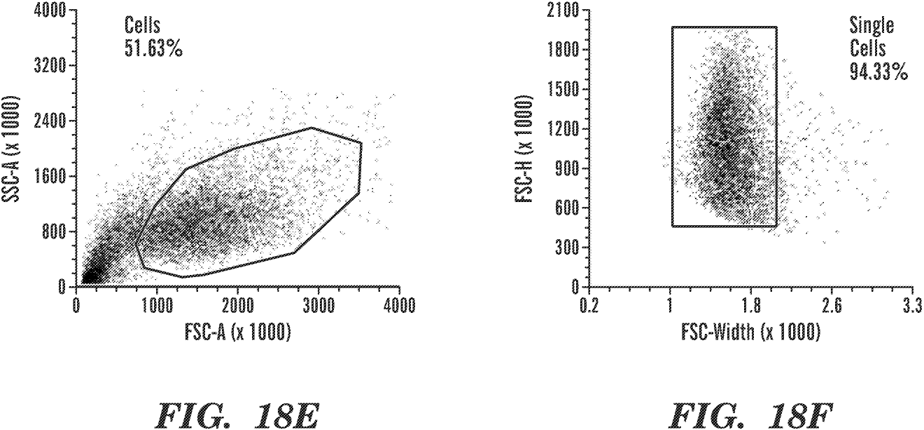

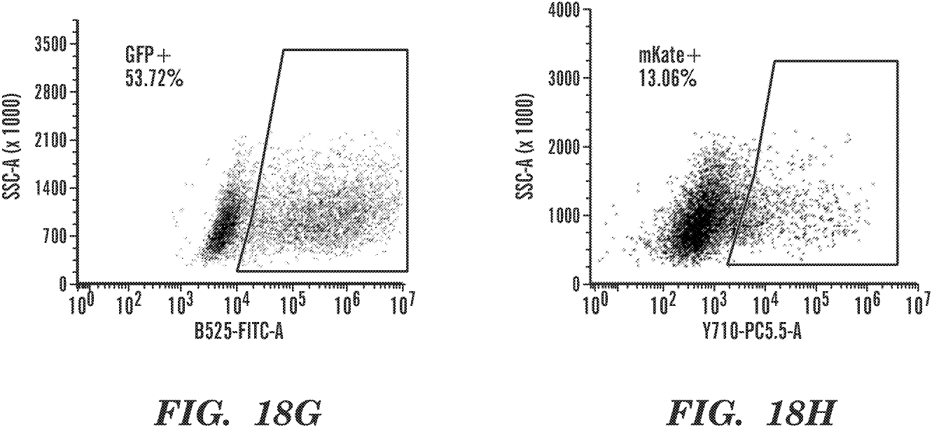

To engineer an RNA-sensing riboswitch that functions in eukaryotic cells, viral IRES modules, which possess structure-guided translational activity¹⁰⁻¹⁴ were modified (FIG. 14B). Although IRESs have been adapted to sense small molecules¹⁵, IRES-based systems have not been previously designed to respond to trRNAs. IRES modules were first selected and tested in a human embryonic kidney 293 (HEK293) cell-based transfection assay (FIG. 17). To avoid 5′ capping, T7 polymerase was co-transfected into cells and used to produce IRES sequences, as these transcripts do not undergo 5′ capping. The presence of IRES modules resulted in a ~9-fold enhancement in expression of in-cis mKate (FIG. 14C, FIGS. 18A-18H). It was decided to pursue cricket paralysis virus (CrPV), kashmir bee virus (KBV), and acute bee paralysis virus (ABPV) IRES modules as the basis for further development of eToeholds, with a focus on the CrPV IRES due to its well-characterized structure and reported functionality in a wide range of eukaryotic systems¹⁰⁻¹².

Having verified IRES activity, it was next hypothesized that inserting short complementary RNA segments into the IRES sequence would disrupt its secondary structure by forming new loops, thereby reducing translational initiation ability, and that breaking of these introduced loops by sense-antisense action of a trRNA would rescue IRES functionality. This hypothesis was tested by inserting reverse complement pieces (two pieces) of DNA into sites within the IRES template, theorizing that base pairing between these sequences in the resulting transcript would distort the functional configuration of the IRES. In order to permit recovery of IRES function upon trRNA presence, these introduced complementary DNA segments were designed to bind to a chosen trRNA sequence. As the goal was for base pairing between the trRNA and the new insertion to be sufficient for breaking the IRES-disrupting loop, insertions were designed to be of unequal lengths. The longer piece (40-50 base pairs) was chosen to be the reverse complement of a portion of the trRNA, while the shorter piece (6-15 base pairs) was chosen to be the reverse complement of a portion of the first piece. Eight sites^{11,16} were selected where insertions, absent modifications to the overall secondary structure, would not erase CrPV IRES activity (FIG. 14D).

Different CrPV IRES sequences with complementary sequences inserted at the eight possible sites were screened. GFP mRNA was chosen to act as the trRNA and the IRES was designed such that GFP mRNA could break apart the newly formed loops. To monitor IRES activity, an in-cis mKate gene downstream of the modified IRES sequences was used, and cells were co-transfected with these constructs as well as a GFP plasmid (FIG. 14E). Each site combination is named with the format: long piece site number-short piece site number (e.g., 1-2). It was found that a number of site combinations (1-2, 1-8, 2-7, 6-7 and 8-6) behaved as expected, producing a higher mKate signal when co-expressing GFP. Based on these results, it was decided to focus on site combinations 6-7 and 8-6, which reproducibly showed 1.7-fold increases in percentage of mKate positive cells in the presence of trigger RNA, which was confirmed via GFP fluorescence.

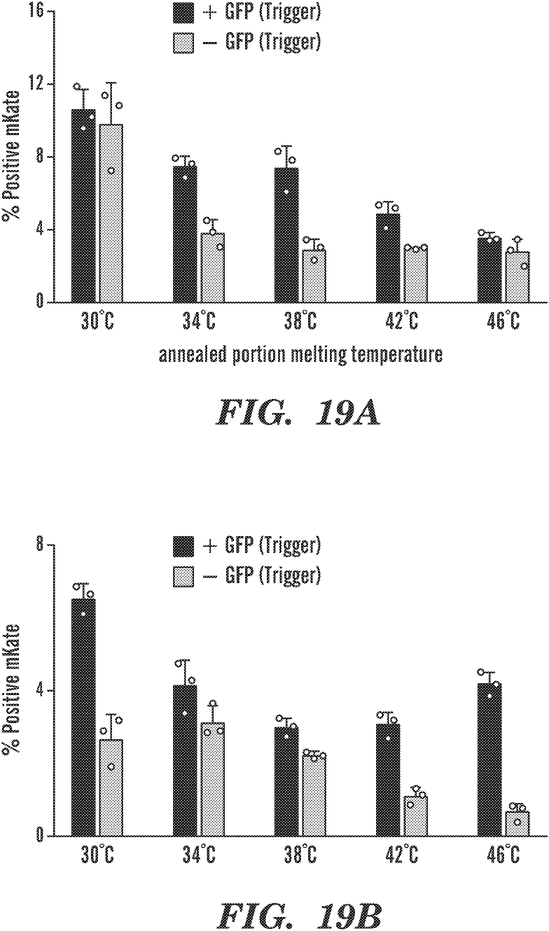

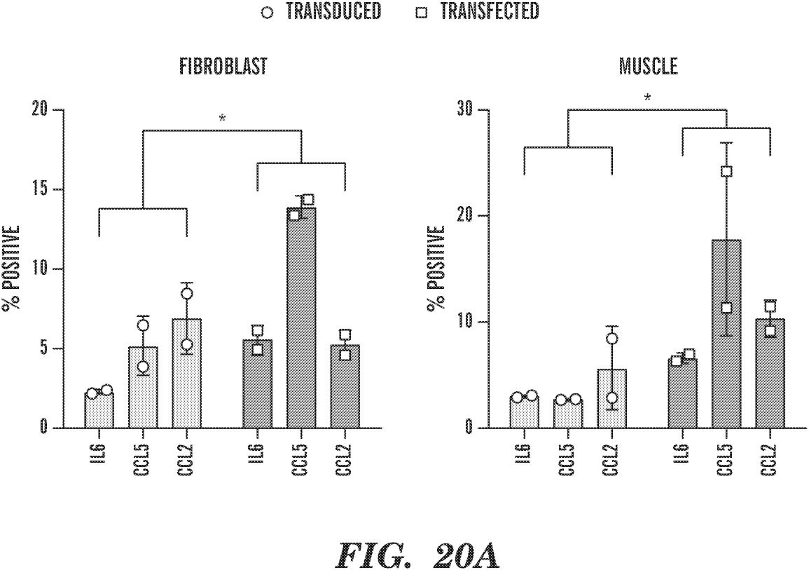

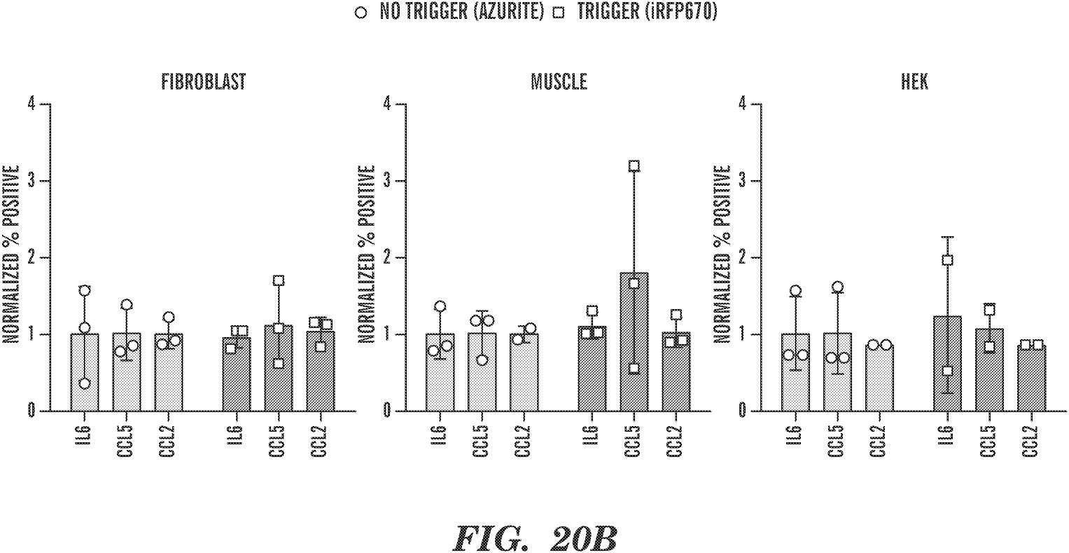

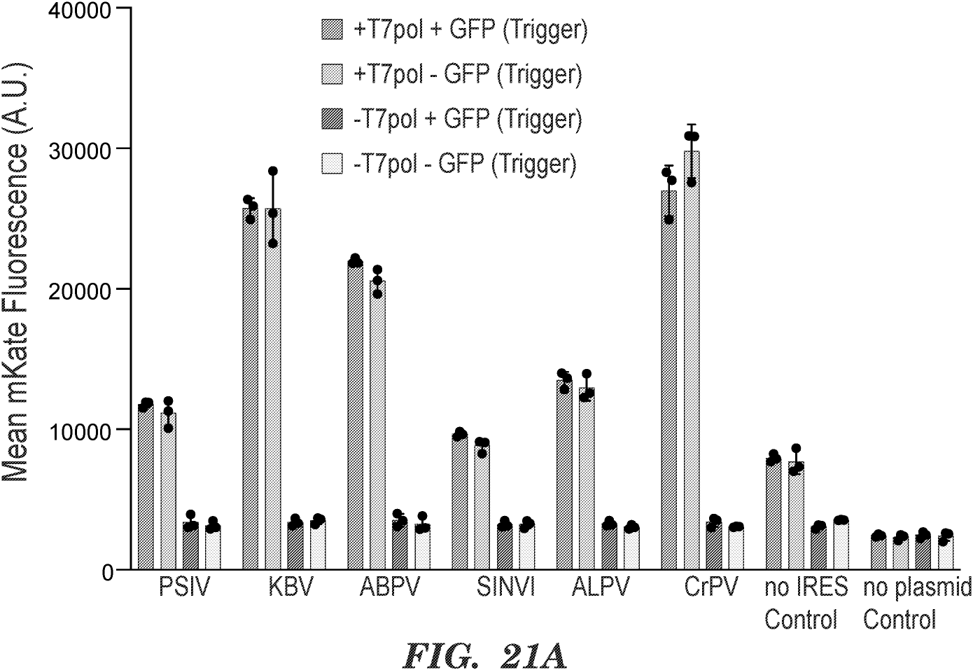

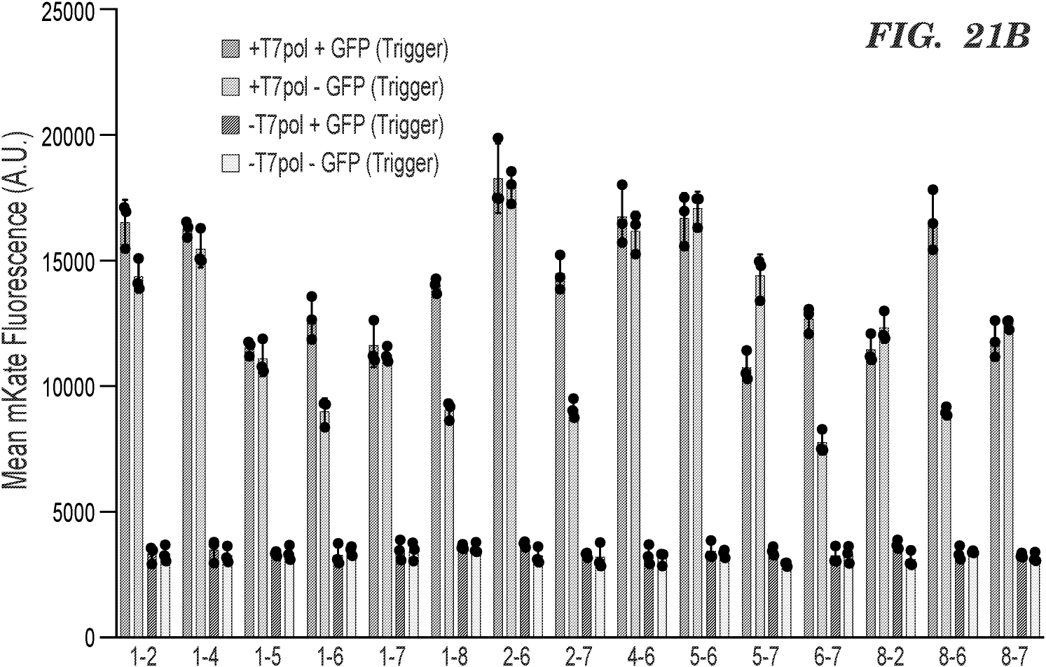

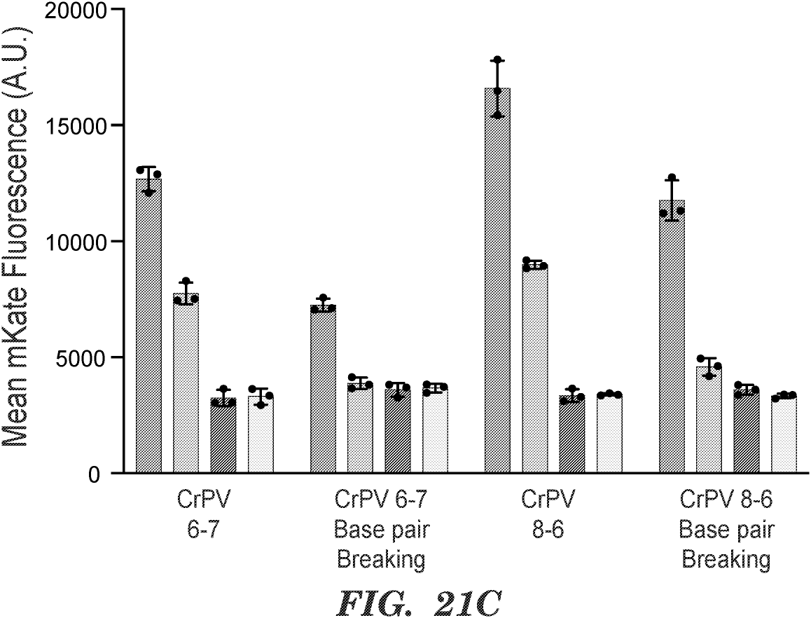

It observed that the modified IRES appeared to retain significant translational ability, despite the newly introduced insertions. IRES pseudoknots might be critical for ribosome recruitment^{10,17}. It was hypothesized that simultaneously distorting the IRES module, as in the above strategy, and breaking the IRES pseudoknot using the same insertion sequences would reduce the basal expression of the module. To test this hypothesis, new eToeholds were designed by choosing the shorter insert to contain sequences present in the pseudoknots. It was theorized that, in the absence of a trigger, the annealing between the inserts would impair correct pseudoknot folding. The insert site where this would occur was termed the base pair breaking site (BB site; FIG. 14F). Designing eToeholds with a BB site led to a reduction of the off (no trigger) state to background levels and increased the on-to-off fold-change from 1.7 to 2.5 for site combination 8-6 (FIG. 14G, FIGS. 21A-21C). In order to further characterize the thermodynamic requirements for eToehold switching, the length of the short insert at the 8-6 site was altered, thus changing annealing temperature. Although some eToeholds displayed a correlation between annealing temperature and output, others did not (FIGS. 19A-19B). As the longer eToehold sequence may be of sufficient length to potentially induce RNAi response, RNA levels of IRES constructs designed to bind to “trigger” RNA sequences were assessed and compared to a nonbinding orthogonal sequence. No significant differences in IRES construct RNA levels were observed in two of the three cell types assessed, but a roughly twofold reduction in the presence of a “trigger” RNA was found in primary human fibroblasts (Table 2). No significant difference was found in the production of cytokines associated with inflammation between cells transduced with “trigger” and orthogonal RNA, although transfection alone significantly increased levels of cytokine production (FIGS. 20A-20B)¹⁸.

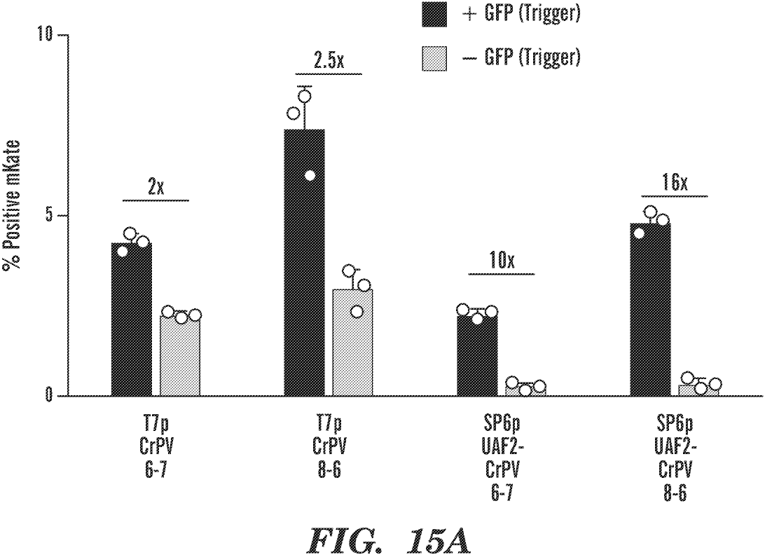

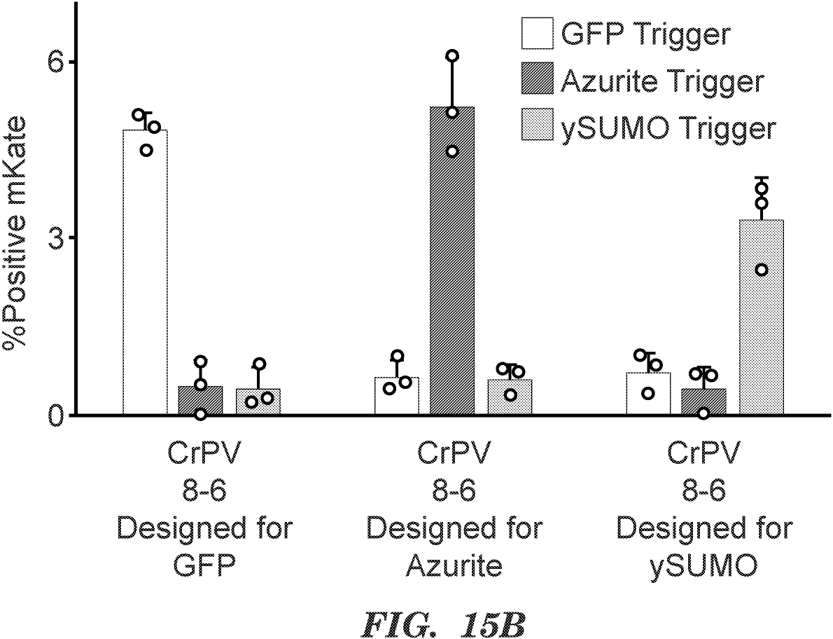

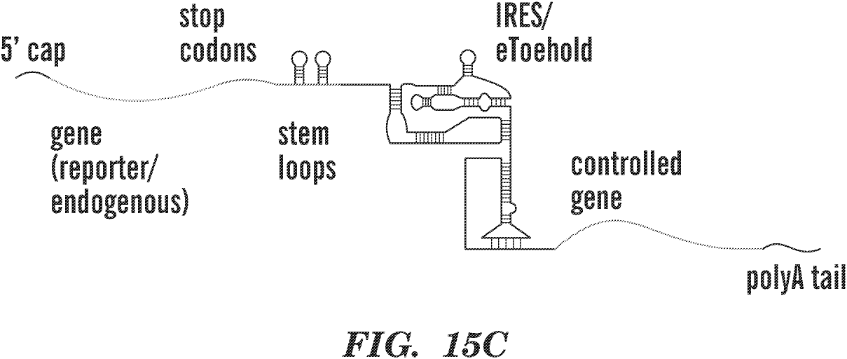

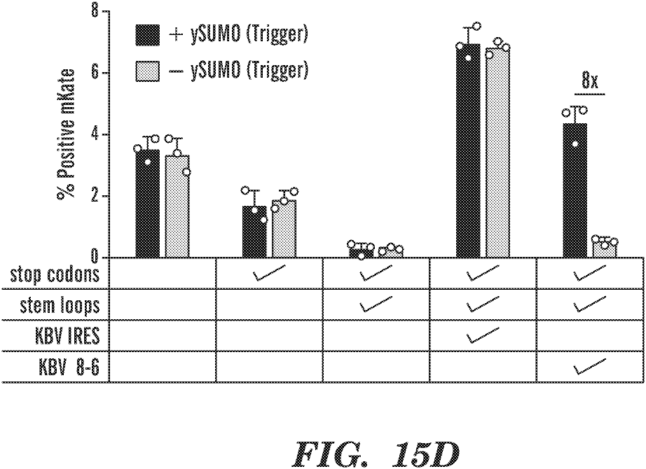

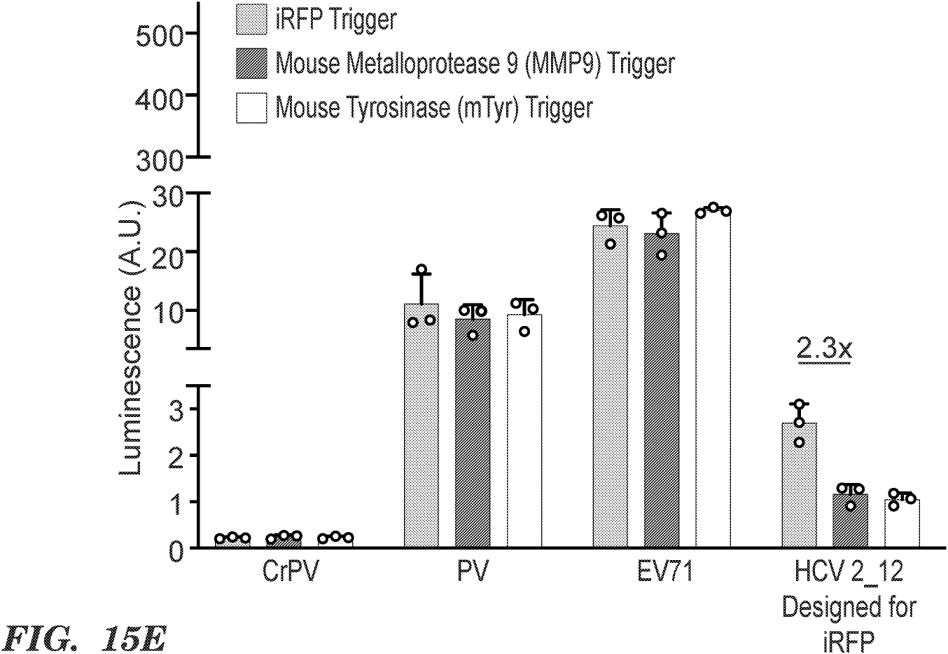

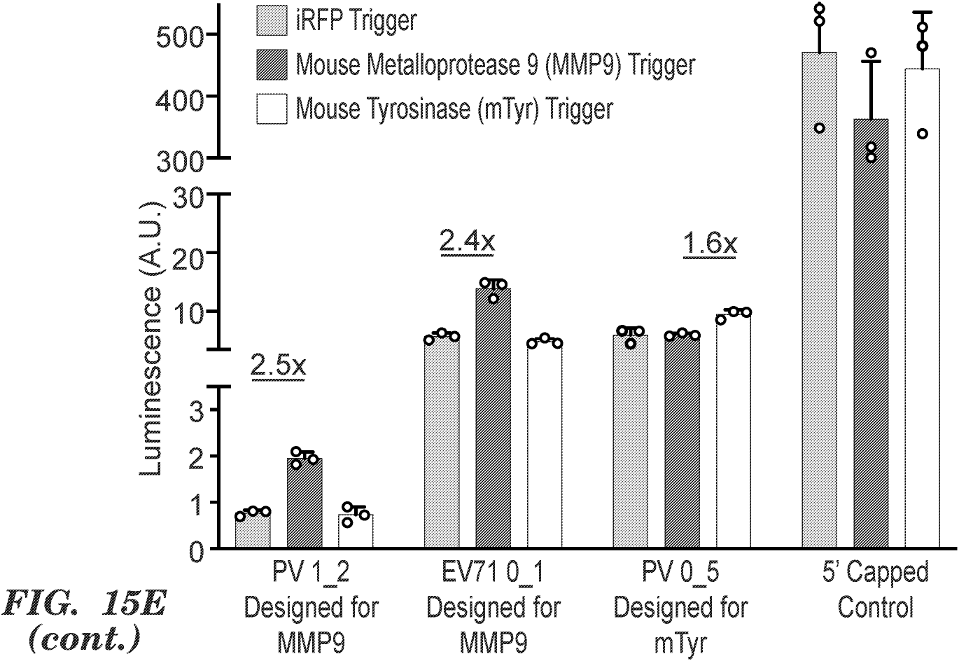

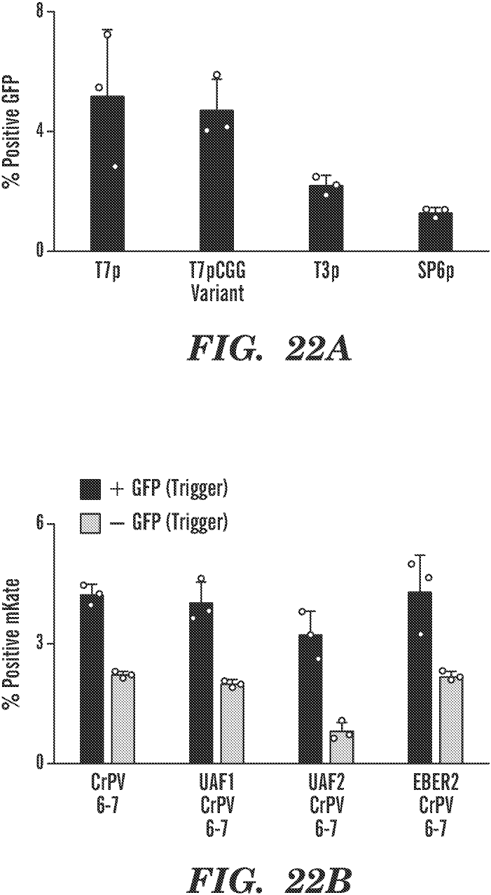

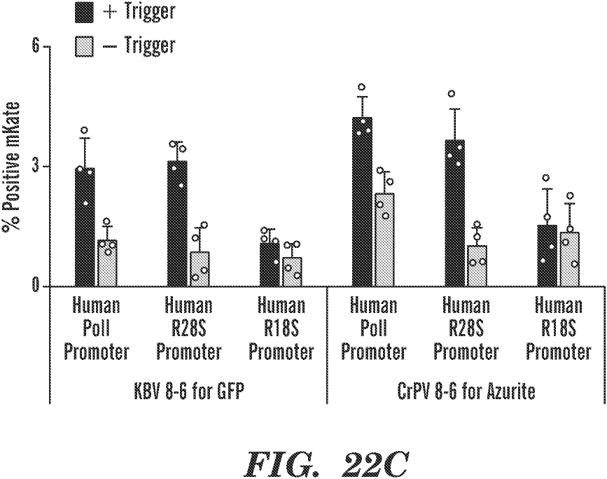

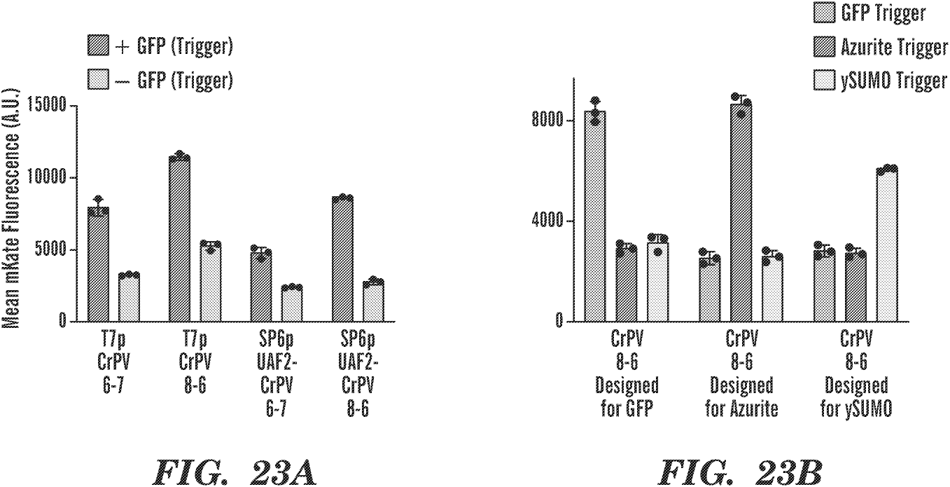

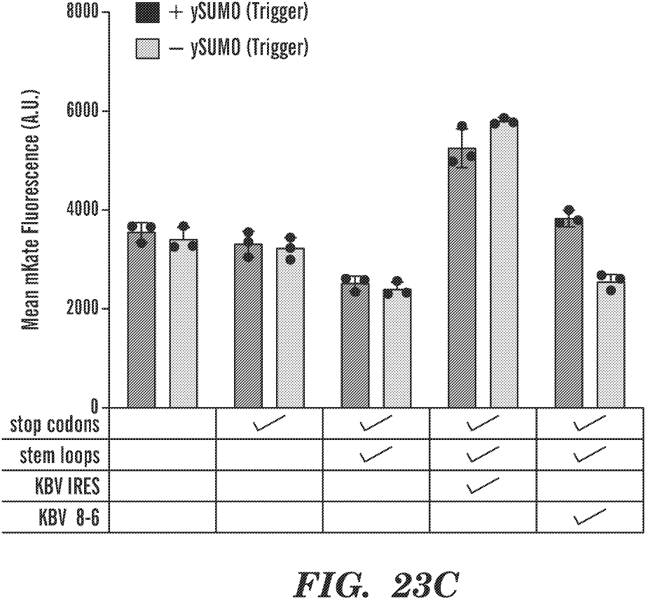

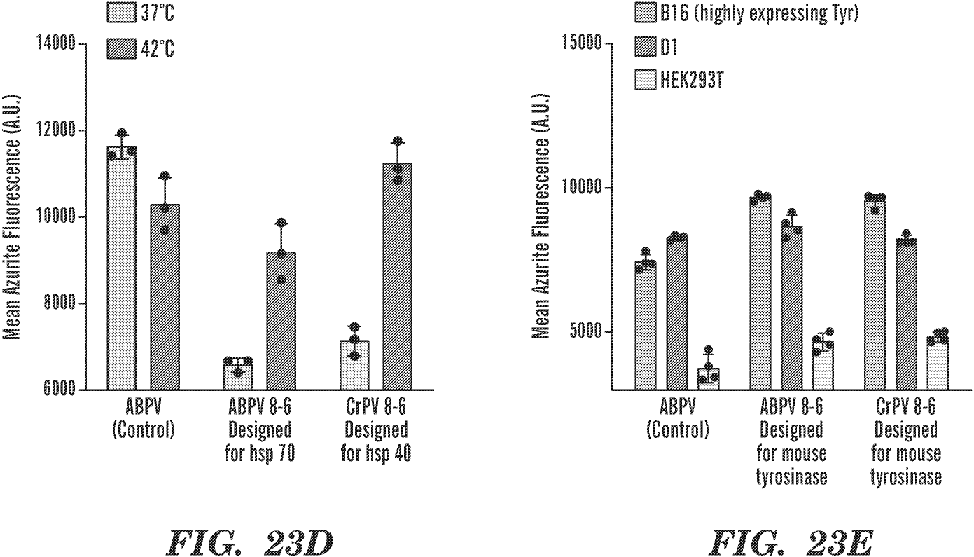

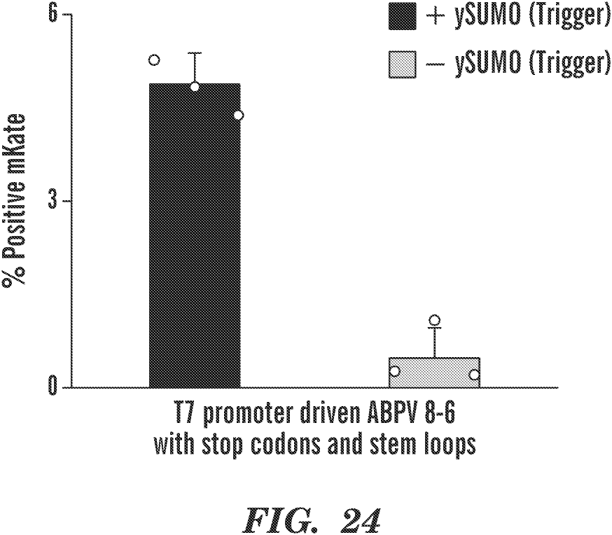

Although T7 promoter (P _{T7}) is generally thought to be highly specific for T7 polymerase, mammalian RNA polymerase II has been shown to bind P _{T7} and initiate significant levels of transcription¹⁹. It was hypothesized that part of the unexpected translation from eToeholds in the absence of trRNA may be due to recruitment of endogenous RNA polymerase II and subsequent generation of transcripts with 5′ caps, which independently induces translation and overrides the need for a functional IRES. Moreover, the recruitment of mammalian RNA polymerase II by P _{T7} may be due to chance similarities in primary sequence between P _{T7} and native mammalian sites of transcriptional initiation. Accordingly, exogenous promoter sequences were screened for transcription systems orthogonal to P _{T7} to test whether off-state translation of IRES-controlled reporter would be reduced. It was found that the promoter for SP6 (P _{SP6}) resulted in significantly lower basal expression than P _{T7} and its analogues (FIG. 22A). Next tested was upstream recruitment of RNA polymerase I, which has been shown to decrease RNA polymerase II binding²⁰⁻²², to test whether this could further decrease basal expression. An upstream activation factor binding DNA sequence from Saccharomyces cerevisiae that successfully reduced basal expression (named UAF2, FIG. 22B) was identified. By combining these components, basal expression was minimized substantially and an on-to-off trigger-mRNA based induction of 15.9-fold was reached (FIGS. 15A and 23A-23E). Similar fold changes upon induction could be achieved by adding stop codons and stem loops²³ before the IRES modules, while retaining the use of the T7 polymerase and promoter (FIG. 24).

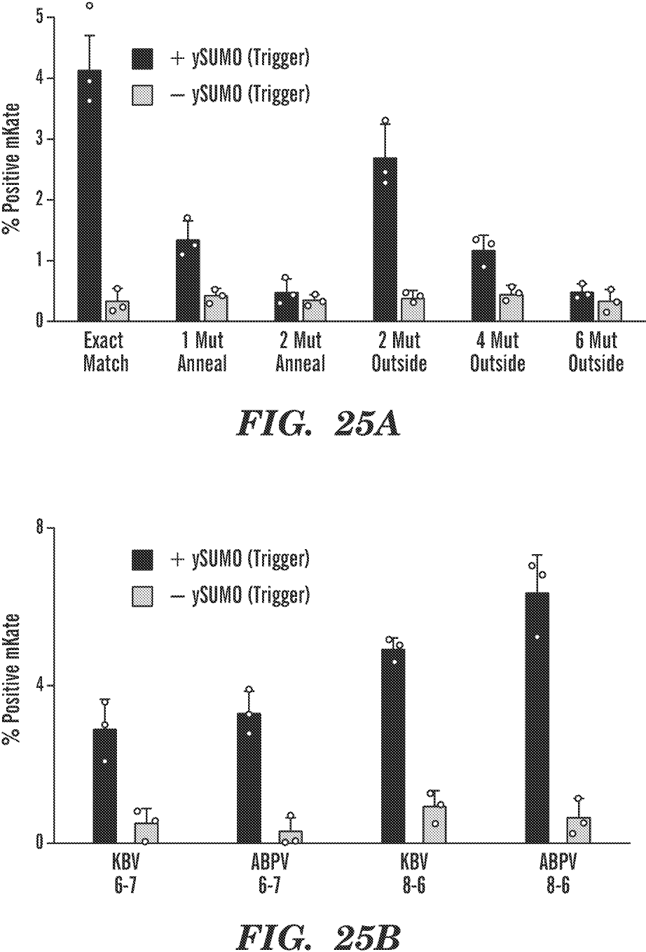

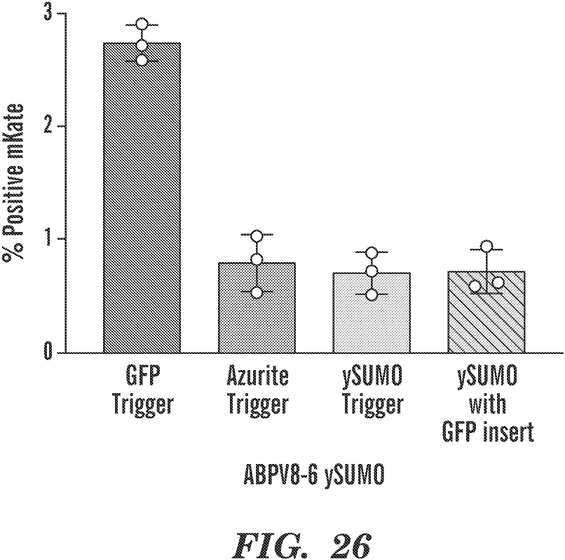

Having developed an approach to achieve robust fold changes in transgene expression upon trRNA induction, the specificity of this system to desired trRNA sequences was next tested. eToeholds were designedto detect GFP, Azurite, and yeast SUMO mRNA, and it was found that these designs specifically sensed their trRNA sequence (FIG. 15B). The sensitivity of eToeholds to their cognate trRNA was further tested by introducing mismatches in the two insertion sequences. It was found that the eToeholds were sensitive to mismatches in the annealing region (FIG. 25A). Additionally, the generalizability of the eToehold design to other IRESs was tested. eToeholds were synthesized using both KBV and ABPV IRES modules and similar fold changes in trigger-induced translation were observed, compared to the CrPV IRES module-based eToeholds (FIG. 25B). Additionally, it was found that RNA sequences that could bind to the short eToehold insertion, but not the longer insertion, were not sufficient for activating the eToehold (FIG. 26). Taken together, these findings indicate that eToeholds can be readily designed for different mRNAs with a high level of specificity, and sense-antisense activation is broadly generalizable for IRES-mediated translation.

It was next sought to adapt the eToehold system to expression by endogenous eukaryotic polymerases. This would obviate dependency on exogenous polymerases for producing uncapped transcripts, and thereby reduce construct size and increase the compatibility of our eToehold system with current cell engineering and gene therapy approaches. Although RNA Polymerase I-responsive promoters^{24,25} have been shown to produce uncapped transcripts, it was found that reliance on RNA polymerase I leads to significantly decreased on-to-off ratios and increased basal expression (FIG. 22C). Therefore, it was decided to explore methods of reducing translational activity in the presence of canonical 5′ capping. By adding stop codons and stem loops²³ after a gene controlled by a constitutive promoter, it was possible to reduce the basal expression of downstream mRNA, despite the reliance on RNA polymerase II for transcription and the presence of 5′ capping (FIGS. 15C, 15D). Furthermore, by inserting an IRES or eToehold module between the stem loops and the coding sequence of a desired gene, creating a bicistronic construct, it was possible to retain trRNA-mediated control over translation. In order to test the applicability of this system in IRES modules that had evolved to utilize mammalian, and specifically human, translational systems, eToehold modules adapted from IRES sequences of hepatitis C virus (HCV)²⁶, poliovirus (PV)²⁷, and enterovirus 71 (EV71) were designed^{28,29}. These new eToeholds also demonstrated an ability to sense specific trRNAs. Compared to the CrPV constructs, these new eToeholds, which were termed human Toeholds (hToeholds), produced more than an order of magnitude more output protein. However, monocistronic 5′ capped mRNA produced higher protein outputs compared to even these new eToeholds, consistent with findings that monocistronic constructs lead to higher levels of mRNA and protein output compared to dicistronic constructs³⁰.

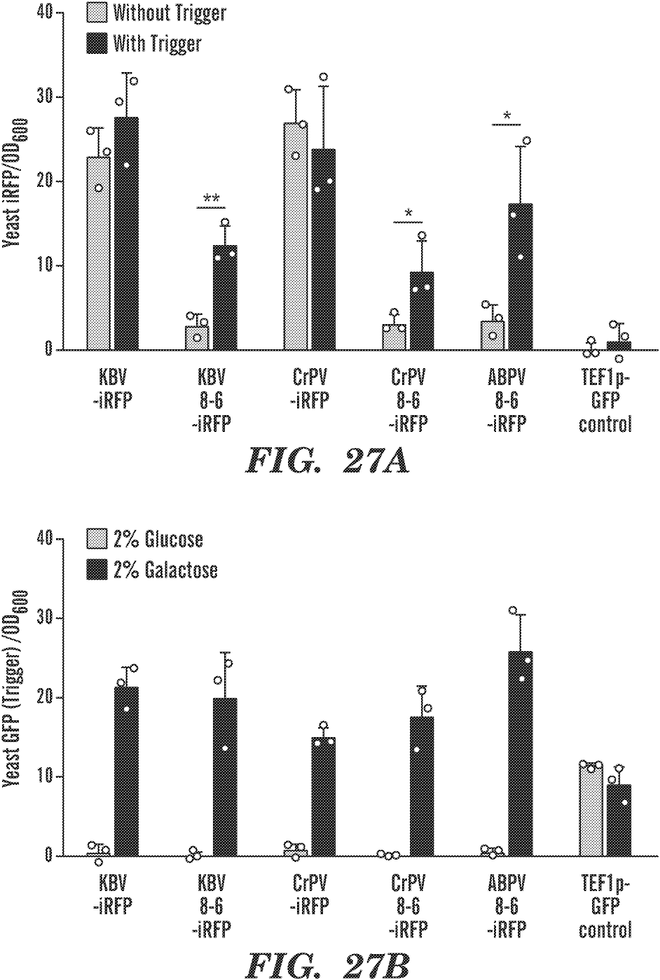

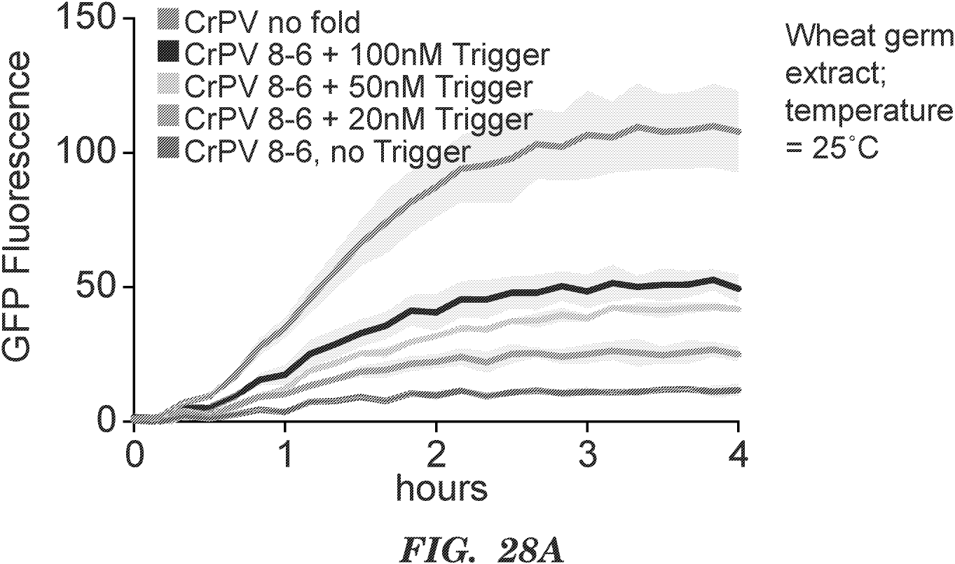

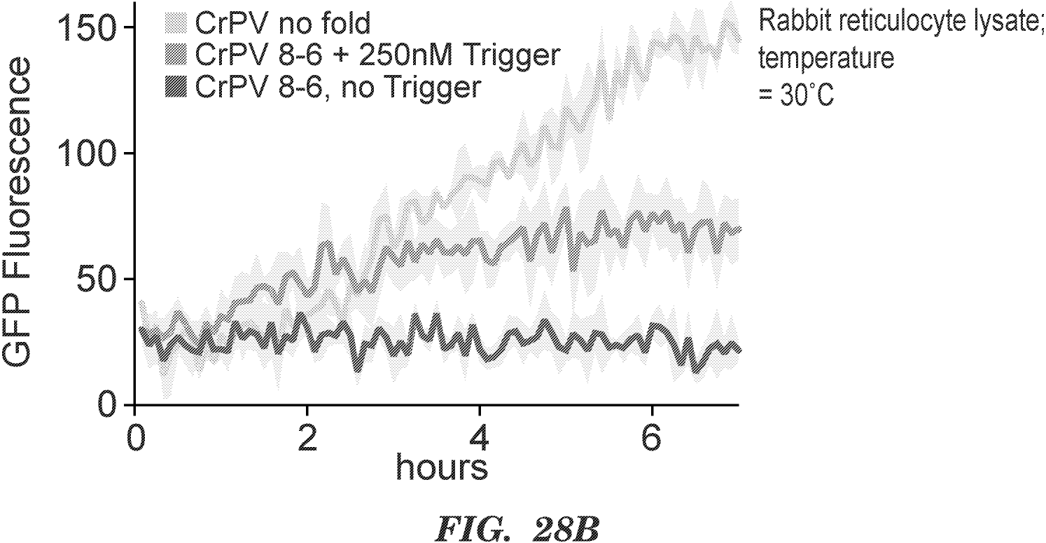

It was next sought to test the functionality of eToehold switches in different eukaryotic systems. To test the eToeholds in a single-cell eukaryote, strains of yeast (Saccharomyces cerevisiae) expressing GFP upon galactose induction, as well as an eToehold designed to produce iRFP670 in the presence of GFP mRNA were created. It was found that although IRES-mediated expression was low, eToeholds were inducible by galactose-controlled GFP expression, while control constructs (unmodified KBV and CrPV, FIGS. 27A-27B) were not. The eToeholds were next tested in eukaryotic cellular extracts (wheat germ and rabbit reticulocyte lysate) to assess their functionality in cell-free systems. Different target RNAs and their matching eToeholds were transcribed, and it was confirmed that eToehold-mediated translation was dependent on the presence of specific triggers (FIGS. 28A-28B). Furthermore, it was observed that this induction was dose-dependent, indicating that eToeholds can provide readouts of relative intracellular RNA levels.

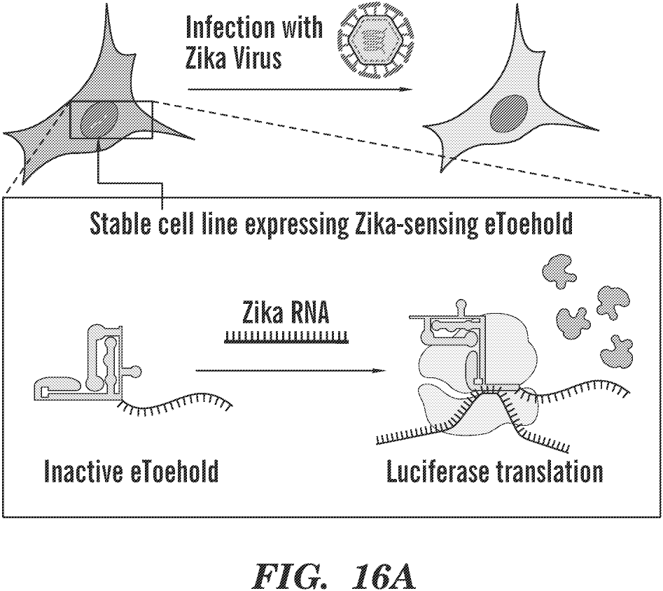

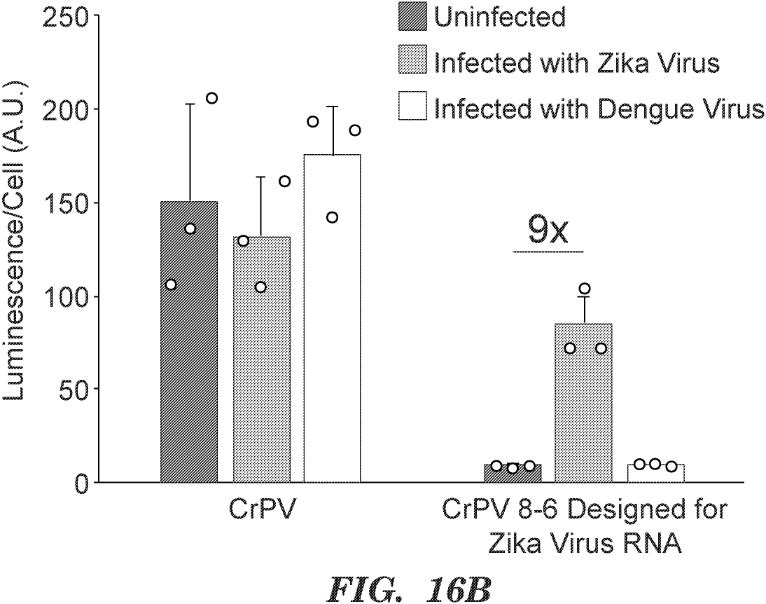

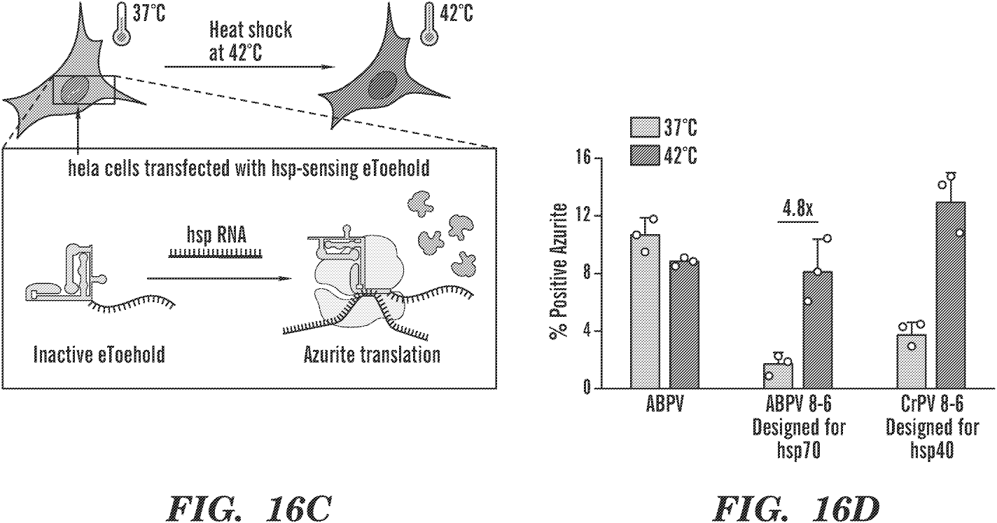

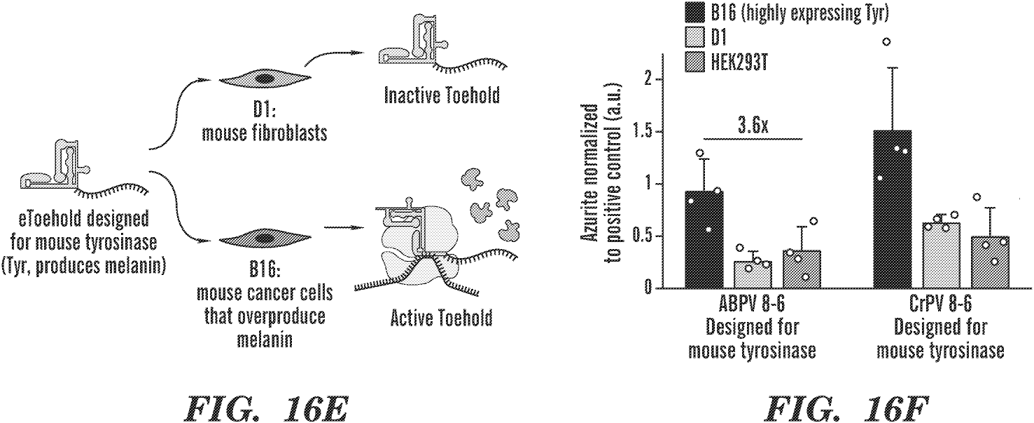

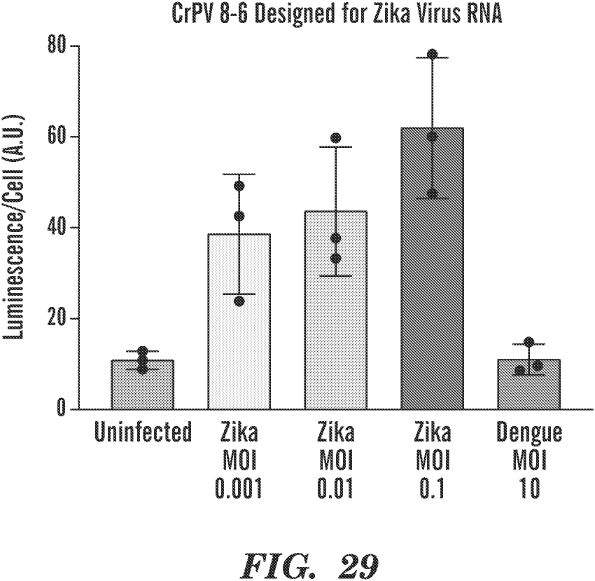

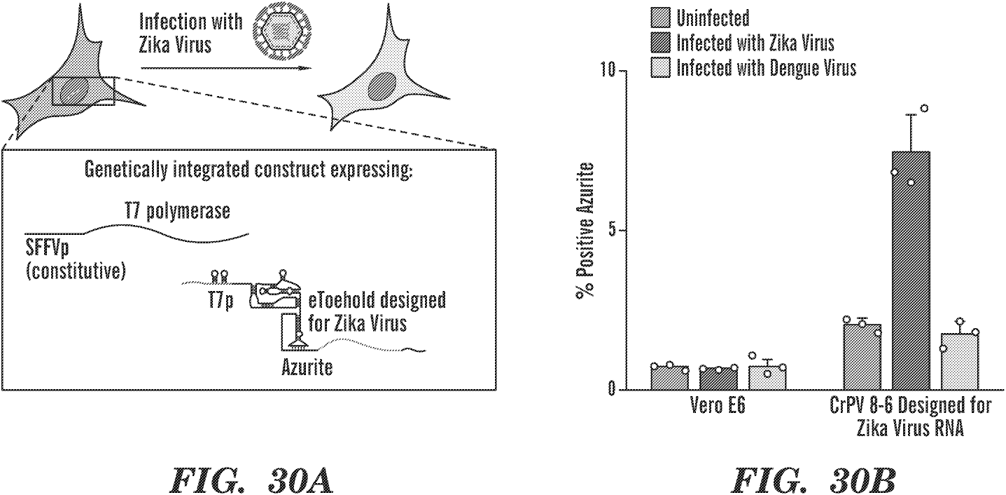

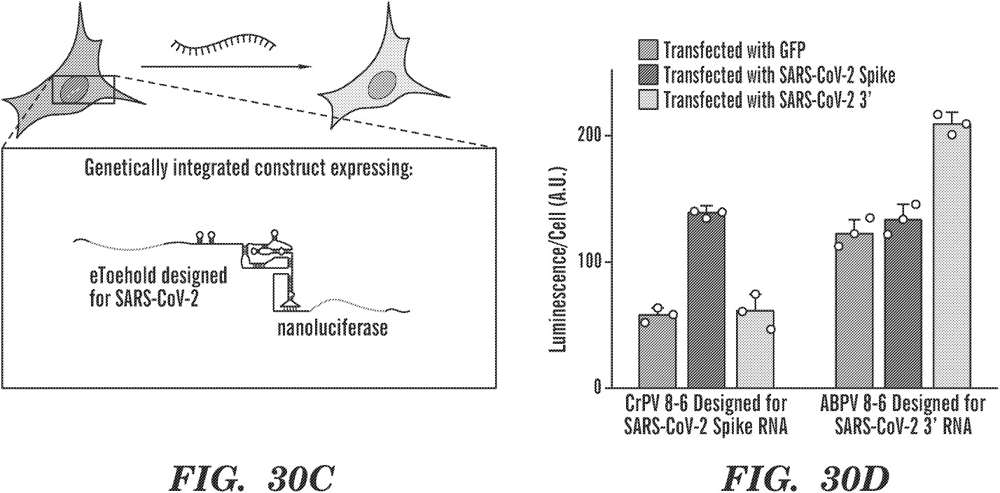

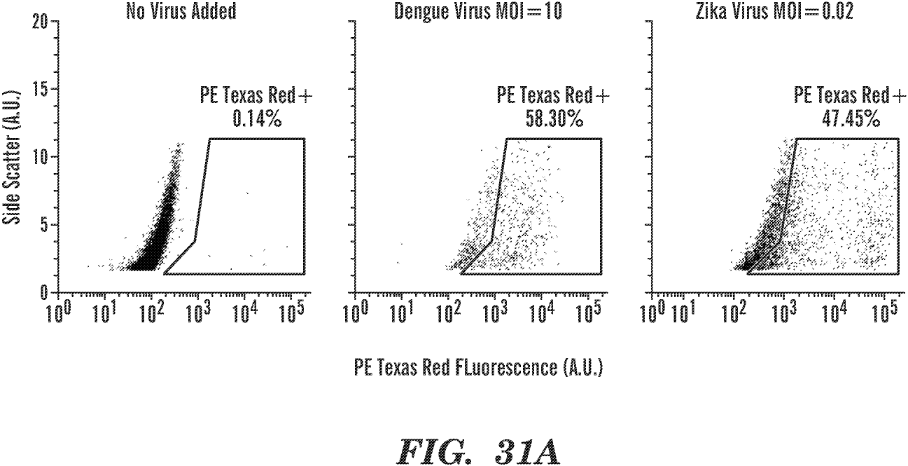

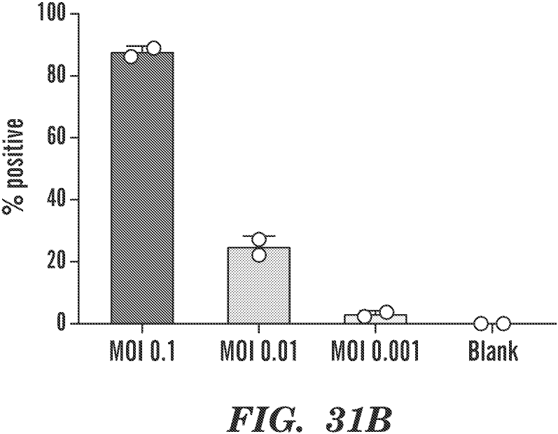

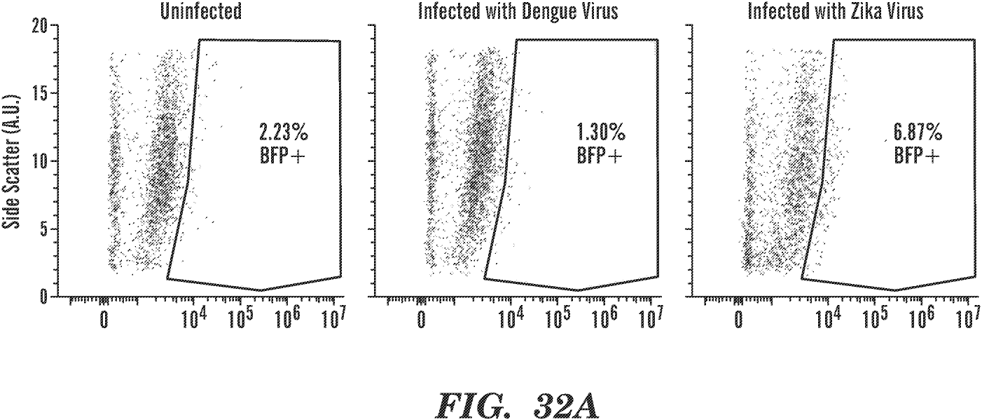

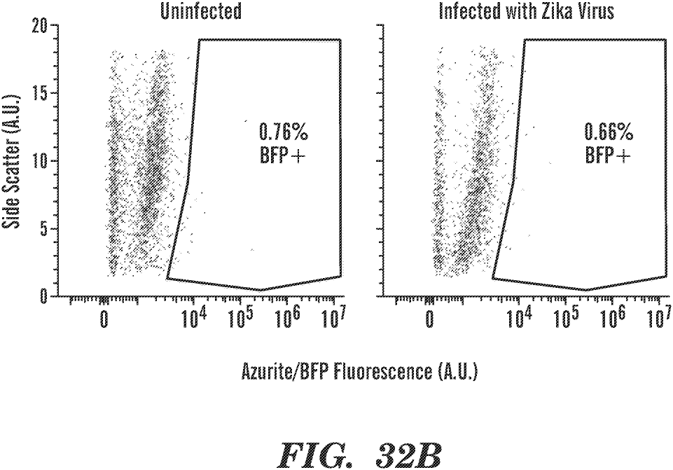

As eToeholds have demonstrated an ability to detect exogenously introduced transcripts in mammalian cells, it was hypothesized that they could serve as live-cell biosensors for viral infection. Lentiviral constructs containing eToeholds specific for Zika and SARS-CoV-2 sequences, respectively, that produced either nanoluciferase or an Azurite fluorescent protein as a readout were generated. These constructs were transduced into the Vero E6 cell line, which is a commonly used host cell for a number of viral diseases. It was found that infection produced an up to 9.2-fold increase in luminescent signal in cells engineered to express Zika-specific eToeholds (FIGS. 16A, 16B), and furthermore demonstrated dose-dependent responsiveness to Zika virus infection at greater sensitivities than existing live cell-biosensor approaches³¹ (FIG. 29). To test the specificity of these eToeholds, infected transduced cells were also with a related Flavivirus virus, Dengue virus³², and it was found that the eToehold response was specific to Zika infection. Similar results were observed using eToeholds encoding an Azurite fluorescent protein (FIGS. 30A, 30B, 31A-31B, and 32A-32B). To test the ability of eToeholds to function as a live-cell sensor for SARS-CoV-2, stable cell lines were engineered with eToeholds designed to sense SARS-CoV-2 transcripts. Upon transfection with constructs expressing fragments of SARS-CoV-2, it was found that these eToehold-engineered cells were able to distinguish between the SARS-CoV-2 trRNAs and other non-target RNAs (FIGS. 30C and 30D).

Finally, the potential of the eToehold system for sensing endogenous transcripts was explored, which would permit applications in discriminating between and targeting of specific cell states and cell types. To assess the ability of eToeholds to determine cell state, eToeholds were designed to produce an Azurite protein reporter in response to transcripts of heat shock proteins hsp70 and hsp40, which are upregulated upon exposure to higher temperatures, in HeLa cells. It was found that the eToehold constructs increased Azurite production up to 4.8-fold after growth at 42° C. for 24 hours, as compared to the routine 37° C. culture (FIGS. 16C, 16D). Next, to assess the ability of eToeholds to differentiate between different cell types, eToeholds were designed to sense mouse tyrosinase (Tyr) mRNA, which is abundant in melanin-forming cells. Again using Azurite as a reporter, a 3.6-fold increase in signal was observed using Tyr-sensing eToeholds transfected into B16-F10 murine melanoma cells, as compared to two control cell lines (HEK293T and D1 marrow stromal cell, FIGS. 16E, 16F). These results indicate that eToeholds can regulate gene and RNA expression based on levels of intracellular, endogenous transcripts, demonstrating their applicability for targeting therapies to specific cell types.

Described herein are eToeholds, a novel RNA-based eukaryotic sense-and-respond module, based on modified IRES elements that permit the translation of a desired protein in the presence of specific RNA trigger sequences. This approach of using sense-antisense interactions to alter secondary structure of IRES modules for translational control surpasses existing RNA-based sensor systems by expanding the length, and therefore specificity, of trigger sequences, and furthermore permits dose-responsiveness to triggers of varying quantity. The proven functionality of eToeholds in multiple domains of eukaryotic life, including fungal and mammalian systems, indicates its potential for broad utility in biotechnology.

Although the eToeholds are RNA-based, DNA transfection and transduction approaches were relied on to produce them intracellularly, as these strategies are widely used in biotechnology settings. In doing so, it was found that incorporating elements to reduce basal mRNA translation is critical for designing RNA-sensing riboswitches. Endogenous 5′ capping exerts a powerful effect on translation, and it was found that using exogenous polymerase systems (T7 or SP6) to transcribe eToeholds in vivo avoided 5′ capping and permitted substantial differences between on and off states. As 5′ capping is beneficial for improving mRNA stability and nuclear export, means to reduce basal translation levels in the presence of 5′ capping were also investigated. Incorporating stem loops and stop codons between the 5′ cap and eToehold module retained 5′ capping but still restricted basal translational levels. RNA-only strategies, whereby pre-transcribed eToehold constructs are transfected directly into cells, can permit greater control over the chemistry and configuration of eToehold molecules, precluding design considerations associated with endogenous transcription processes.

eToeholds can detect a variety of intracellular RNAs, including those introduced exogenously by transfection or infection, and endogenous transcripts, such as those indicative of cell state or cell type. The ability to initiate translation of a desired protein in response to the presence of cell-type- or cell-state-specific RNA transcripts, as demonstrated here with eToeholds, has significant potential in improving biotechnology therapies. Although many molecules hold therapeutic promise, their clinical utility is frequently hampered by severe off-target toxicities. The ability of an eToehold module to translate a protein or protein-based precursor in response to an mRNA signature may make it a highly useful tool for addressing this challenge, by restricting the activation of a desired therapy to specific target cells. It is contemplated herein that eToehold designs implementing logic gates permit computing cell states from multiple trRNAs, further enhancing the specificity and utility of this system.

### Materials and Methods

Assembly of DNA constructs. Promoter-gene-polyA tail sequences were cloned into pCAG-T7pol (Addgene #59926) or pXR1. pCAG-T7pol based plasmids were cut with EcoRI and NotI at the 5′ and 3′ ends, respectively, to insert genes for the replacement of T7 polymerase. pXR1 was cut with NotI and NcoI to replace IRES modules. pXR1 was cut with PacI and BglIIto replace promoter sequences. pXR1 was cut with NcoI and XhoI to replace the reporter gene. Lentiviral vectors were cloned from pLenti CMV Puro DEST (Addgene #17452). pLenti CMV Puro DEST was cut with BspDI and PshAI to change promoters (specifically to P_{SFFV}). pLenti CMV Puro DEST was cut with PshAI and XmaI to change reporter genes. pLenti CMV Puro DEST was cut with XmaI and SalI to add the eToehold-controlled gene construct. All eToehold constructs were created such that the downstream gene was in frame with the noncanonical start codon within the IRES element³³. Yeast plasmids were assembled as previously described³⁴.

Qiagen Miniprep or Qiagen Gel Extraction purification kits were used to extract and purify plasmids or fragments. All insertions were either ordered as a gBlock gene fragment from Integrated DNA Technologies or amplified from laboratory plasmids or human genomic DNA through PCR to include homology arms for Gibson isothermal assembly (using a 2x reaction mix purchased from New England Biosciences). All vectors were sequenced using Sanger sequencing from GENEWIZ before experimentation. Tandem repeats were avoided to prevent recombination events.

HEK293T cell transfection and construct testing. Plasmid concentrations were determined using a NanoDrop OneC™ Microvolume UV-Vis Spectrophotometer. For eToehold construct testing, transfections were performed using Lipofectamine 3000™ Transfection Reagent and standard protocols in a 96-well plate of 70% confluent HEK293T cells. For each well, 30 ng of T7/SP6 polymerase-expressing construct, 70 ng of eToehold-mKate construct, and 50 ng of trigger RNA-expressing construct were transfected. For samples that did not require T7/SP6 polymerase, 75 ng of eToehold constructs and 75 ng of trigger RNA-expressing construct were added. After 60 hours of incubation at 37° C., cells were detached using TripLE Express™ and resuspended in 2% FBS in PBS for flow cytometry in a Cytoflex LX™ flow cytometer.

Yeast transformation and experimentation. Yeast transformations were carried out as previously described³⁴. EZ-L1 was cut with PmeI and transformed into CEN.PK2-1C to generate strain EZy1. EZy1 was then transformed with EZ-L183, EZ-L184, EZ-L185, EZ-L186, and EZ-L187 to generate strains EZy13, EZy14, EZy15, EZy16, and EZy17, respectively. Liquid yeast cultures were grown in 24-well plates, at 30° C. and shaken at 200 rpm in SC-dropout medium supplemented with 2% glucose and 50 ng/mL biliverdin (from VWR International, LLC for iRFP imaging). Fluorescence and OD600 measurements were taken utilizing the Biotek Synergy H1™ Plate Reader; GFP fluorescence measurements used excitation and emission wavelengths of 485 nm and 535 nm, respectively, and iRFP fluorescence measurements used excitation and emission wavelengths of 640 nm and 675 nm, respectively. Growth and fluorescence normalization were performed as previously described³⁴.

Cell-free lysate testing. RNA was generated using constructs bearing T7 promoters preceding desired RNA (EZ-L212, EZ-L214, EZ-L366) and HiScribe™ T7 High Yield RNA Synthesis Kit by New England Biolabs, according to the manufacturer’s instructions (including DNaseI treatment). After purification using a Zymo Research RNA Clean and Concentrator Kit, RNA was added to wheat germ extract from Promega or reticulocyte lysate IVT Kit from ThermoFisher Scientific, according to the manufacturers’ instructions. Changes in fluorescence were measured over 6 hours using a Biotek Synergy H1™ Plate Reader.

Lentivirus generation and transduction. Lentiviruses were generated as previously described³⁵, using psPAX2 and pMD2.G as helper plasmids and cloning the transfer vector based on pLenti CMV Puro DEST (w118-1). psPAX2 was a gift from Didier Trono (Addgene plasmid # 12260 ; available on the world wide web at n2t.net/addgene:12260 ; RRID:Addgene_12260). pMD2.G is Addgene plasmid # 12259 ; available on the world wide web at n2t.net/addgene:12259 ; RRID:Addgene_12259. pLenti CMV Puro DEST (w118-1) is Addgene plasmid # 17452; available on the world wide web at n2t.net/addgene:17452; RRID:Addgene_17452. EZ-L521, EZ-L534 and EZ-L536 were cloned from pLenti CMV Puro DEST (w118-1) through Gibson assembly. After transduction of Vero E6 cells with the lentiviruses, transduced cells were sorted for GFP fluorescence using a Sony SH800™ cell sorter.

Zika virus infection testing. Vero E6 cells (maintained in DMEM 10% FBS), Dengue virus serotype 2 (DENV2 strain New Guinea C, Accession AAA42941), and Zika virus isolates (ZV, Pernambuco isolate 243, Accession MF352141) were used. Cell lines were plated and grown overnight to 90% confluent monolayers and infected with ZV (at MOI = 0.02 for stable cell line created using EZ-L536 or EZ-L521, and at MOI = 2 for stable cell line created using EZ-L534, in DMEM 2% FBS) or DENV2 (at MOI = 2, in DMEM 2% FBS)³², and fixed at 48 hpi using Cytofix™ from BD sciences and subsequently washed twice in DPBS 2% FBS. Replicates of cells were fixed and stained with anti-NS1 antibodies 7724.323 (diluted 1:1000 anti-Dengue NS1 mAb 323); 7944.644 (diluted 1:2000; anti-Zika NS1 mAb 644)^{36,37}. Other replicates of cells were run on a CytoFlex LX™ flow cytometer or utilized for a nanoluciferase assay using Nano-Glo™ from Promega, according to the manufacturer’s instructions and measured using a Biotek Synergy H1™ Plate Reader.

Heat shock-sensing eToehold testing. HeLa cells were transfected with EZ-L512 (ABPV positive control), EZ-L548 (an eToehold that senses hsp70), or EZ-L554 (an eToehold that sense hsp40). Thirty-six hours post-transfection, a portion of the samples was moved to 42° C. for 24 hours. Cells were detached using TripLE™ Express and resuspended in 2% FBS in PBS for flow cytometry in a Cytoflex LX™ flow cytometer.

Mouse Tyr eToehold testing. B16-F10 melanoma cells and D1 marrow stromal cells, as well as HEK293T cells (ATCC), were maintained at subconfluency in DMEM 10% FBS. Cells were transfected with plasmids encoding eToeholds or control sequences using Mirus TransIT-2020™ transfection reagent, per the manufacturer’s instructions (Mirus Bio). Forty-eight hours after transfection, cells were detached, stained for viability (Fisher Scientific), and analyzed using flow cytometry with a LSRFortessa™ with HTS (BD Biosciences). FACSDiva™ software (BD Biosciences) was used for analysis of samples. Only viable cells were included for analysis.

Intracellular cytokine staining. HEK293T, human skeletal muscle cells (ATCC), and human dermal fibroblast cells (ATCC) were cultured in DMEM 10% FBS, Primary Skeletal Muscle Growth Kit in Mesenchymal Stem Cell Basal Media (ATCC), and Fibroblast Growth Media 2 (Promocell), respectively. Cells were transfected or transduced with EZ-L793 to introduce eToehold, and either EZ-L1061 or EZ-L1062. Either 24 hours after transfection or after antibiotic selection after transduction, cells were fixed and permeabilized with Cytofix/Cytoperm™ (BD Biosciences); stained with either PE/Dazzle 594 anti-human IL-6 (501121), PE anti-human CCL5 (515503), or PE anti-human CCL2 (505903); and analyzed with a LSRFortessa™ with HTS (BD Biosciences). FACSDiva™ software (BD Biosciences) was used for analysis of samples.

qPCR. RNA was isolated after cells were pelleted and frozen by using a RNEasy™ Plus Mini kit (Qiagen), following manufacturer’s instructions, and stored at -80° C. or immediately used. RNA was quantified by NanoDrop™ spectrophotometer, and reverse transcription and amplication was performed using a Luna Universal One-Step RT-qPCR kit (New England BioLabs) on a CFX96™ RT-PCR machine (Bio-Rad). Either PrimePCR primers (Bio-Rad; qHsaCED0038674 for human glyceraldehyde-3-phosphate dehydrogenase, GAPDH) or custom primers (5′ TTGACGGAGTCACACCGAAT 3′ (SEQ ID NO: 37) and 5′ GTCACTCTGAACAGGAGGCT 3′ (SEQ ID NO: 38) for nanoluciferase) were used. Custom primers were validated by calculating primer efficiency from a standard curve of nanoluciferase template RNA. Relative gene expression was computed by the delta-delta Ct method, which compared Ct values to a control sample (orthogonal trigger) and reference gene (GAPDH).

Statistics. Statistical significance was determined using a standard t-test for calculating p values. T scores were calculated by the formula:

$$\frac{\left(M e a {n}_{C o n d i t i o n 2} - M e a {n}_{C o n d i t i o n 1}\right) \sqrt{N u m b e r o f s a m p l e s}}{S t a n d a r d D e v i a t i o {n}_{C o n d i t i o n 2}} .$$

P values were calculated using two degrees of freedom and a one-sided t-test calculator. For qPCR, statistics were performed delta-Ct values, which are the relative expression normalized to reference gene GAPDH, in PRISM using a 2-way ANOVA followed by Sidak multiple comparisons testing.

### REFERENCES

1. Green, A. A., Silver, P. A., Collins, J. J. & Yin, P. Toehold switches: De-novo-designed regulators of gene expression. Cell 159, 925-939 (2014).

2. Hanewich-Hollatz, M. H., Chen, Z., Hochrein, L. M., Huang, J. & Pierce, N. A. Conditional Guide RNAs: Programmable Conditional Regulation of CRISPR/Cas Function in Bacterial and Mammalian Cells via Dynamic RNA Nanotechnology. ACS Cent. Sci. 5, 1241-1249 (2019).

3. Siu, K. H. & Chen, W. Riboregulated toehold-gated gRNA for programmable CRISPR-Cas9 function. Nat. Chem. Biol. 15, 217-220 (2019).

4. Kim, J. et al. De novo-designed translation-repressing riboregulators for multi-input cellular logic. Nat. Chem. Biol. 15, 1173-1182 (2019).

5. Wang, S., Emery, N. J. & Liu, A. P. A Novel Synthetic Toehold Switch for MicroRNA Detection in Mammalian Cells. ACS Synth. Biol. 8, 1079-1088 (2019).

6. Litke, J. L. & Jaffrey, S. R. Highly efficient expression of circular RNA aptamers in cells using autocatalytic transcripts. Nat. Biotechnol. 37, 667-675 (2019).

7. Felletti, M., Stifel, J., Wurmthaler, L. A., Geiger, S. & Hartig, J. S. Twister ribozymes as highly versatile expression platforms for artificial riboswitches. Nat. Commun. 7, 2-9 (2016).

8. Zhong, G., Wang, H., Bailey, C. C., Gao, G. & Farzan, M. Rational design of aptazyme riboswitches for efficient control of gene expression in mammalian cells. Elife 5, (2016).

9. Zhong, G. et al. A reversible RNA on-switch that controls gene expression of AAV-delivered therapeutics in vivo. Nat. Biotechnol. doi:10.1038/s41587-019-0357-y

10. Ruehle, M. D. et al. A dynamic RNA loop in an IRES affects multiple steps of elongation factor-mediated translation initiation: (A) Schematic of the IGR IRES initiation factor-independent translation initiation mechanism. The IGR IRESs occupy the same binding sites as tRNAs i. Elife 4, 1-24 (2015).

11. Kanamori, Y. & Nakashima, N. A tertiary structure model of the internal ribosome entry site (IRES) for methionine-independent initiation of translation. Rna 7, 266-274 (2001).

12. Zhu, J. et al. Crystal structures of complexes containing domains from two viral internal ribosome entry site (IRES) RNAs bound to the 70S ribosome. Proc. Natl. Acad. Sci. U. S. A. 108, 1839-1844 (2011).

13. Yamamoto, H., Nakashima, N., Ikeda, Y. & Uchiumi, T. Binding mode of the first aminoacyl-tRNA in translation initiation mediated by Plautia stali intestine virus internal ribosome entry site. J. Biol. Chem. 282, 7770-7776 (2007).

14. Colussi, T. M. et al. Initiation of translation in bacteria by a structured eukaryotic IRES RNA. Nature 519, 110-113 (2015).

15. Ogawa, A., Masuoka, H. & Ota, T. Artificial OFF-Riboswitches That Downregulate Internal Ribosome Entry without Hybridization Switches in a Eukaryotic Cell-Free Translation System. ACS Synth. Biol. 6, 1656-1662 (2017).

16. Hodgman, C. E. & Jewett, M. C. Characterizing IGR IRES-mediated translation initiation for use in yeast cell-free protein synthesis. N. Biotechnol. 31, 499-505 (2014).

17. Nishiyama, T. et al. Structural elements in the internal ribosome entry site of Plautia stali intestine virus responsible for binding with ribosomes. Nucleic Acids Res. 31, 2434-2442 (2003).

18. Wesselhoeft, R. A. et al. RNA Circularization Diminishes Immunogenicity and Can Extend Translation Duration In Vivo. Mol. Cell 74, 508-520.e4 (2019).

19. Sandig, V., Lieber, A., Bahring, S. & Strauss, M. A phage T7 class-III promoter functions as a polymerase II promoter in mammalian cells. Gene (1993). doi:10.1016/0378-1119(93)90302-J

20. Bensaude, O. Inhibiting eukaryotic transcription. Transcription 2, 103-108 (2011).

21. Keys, D. A. et al. Multiprotein transcription factor UAF interacts with the upstream element of the yeast RNA polymerase I promoter and forms a stable preinitiation complex. Genes Dev. 10, 887-903 (1996).

22. Vu, L., Siddiqi, I., Lee, B. S., Josaitis, C. A. & Nomura, M. RNA polymerase switch in transcription of yeast rDNA: Role of transcription factor UAF (upstream activation factor) in silencing rDNA transcription by RNA polymerase II. Proc. Natl. Acad. Sci. U. S. A. 96, 4390-4395 (1999).

23. Bao, C. et al. mRNA stem-loops can pause the ribosome by hindering A-site tRNA binding. Elife 9, 1-67 (2020).

24. Hoffmann, E., Neumann, G., Hobom, G., Webster, R. G. & Kawaoka, Y. “Ambisense” approach for the generation of influenza A virus: vRNA and mRNA synthesis from one template. Virology 267, 310-317 (2000).

25. Czudai-Matwich, V., Schnare, M. & Pinkenburg, O. A simple and fast system for cloning influenza A virus gene segments into pHW2000- and pCAGGS-based vectors. Arch. Virol. 158, 2049-2058 (2013).

26. Yokoyama, T. et al. HCV IRES Captures an Actively Translating 80S Ribosome. Mol. Cell 74, 1205--1214.e8 (2019).

27. Ochs, K. et al. Interaction of Translation Initiation Factor eIF4B with the Poliovirus Internal Ribosome Entry Site. J. Virol. (2002). doi:10.1128/jvi.76.5.2113-2122.2002

28. Thompson, S. R. & Sarnow, P. Enterovirus 71 contains a type I IRES element that functions when eukaryotic initiation factor eIF4G is cleaved. Virology 315, 259-266 (2003).

29. Davila-Calderon, J. et al. IRES-targeting small molecule inhibits enterovirus 71 replication via allosteric stabilization of a ternary complex. Nat. Commun. 11, (2020).

30. Sadikoglou, E., Daoutsali, E., Petridou, E., Grigoriou, M. & Skavdis, G. Comparative analysis of internal ribosomal entry sites as molecular tools for bicistronic expression. J. Biotechnol. 181, 31-34 (2014).

31. McFadden, M. J. et al. A fluorescent cell-based system for imaging zika virus infection in real-time. Viruses 10, 13-18 (2018).

32. Medina, F. et al. Dengue virus: Isolation, propagation, quantification, and storage. Curr. Protoc. Microbiol. (2012).doi:10.1002/9780471729259.mc15d02s27

33. Murray, J. et al. Structural characterization of ribosome recruitment and translocation by type IV IRES. Elife (2016).doi:10.7554/eLife.13567

34. Zhao, E. M. et al. Optogenetic regulation of engineered cellular metabolism for microbial chemical production. Nature 555, 683-687 (2018).

35. Campeau, E. et al. A versatile viral system for expression and depletion of proteins in mammalian cells. PLoS One (2009).doi:10.1371/journal.pone.0006529

36. Bosch, I. et al. Serotype-specific detection of dengue viruses in a nonstructural protein 1-based enzyme-linked immunosorbent assay validated with a multi-national cohort. PLoS Negl. Trop. Dis. (2020).doi:10.1371/journal.pntd.0008203

37. Bosch, I. et al. Rapid antigen tests for dengue virus serotypes and zika virus in patient serum. Sci. Transl. Med. (2017).doi:10.1126/scitranslmed.aan1589

TABLE 1

Plasmids used in this study

| | | | |
|---|---|---|---|
| Plasmid Name | Content 1 | Content 2 | Use |
| pCAG-T7pol | P_{CAGGS}-T7polymerase-PolyA_{□}_{Globin} | - | T7 polymerase expression for testing |
| pCAG-SP6pol | P_{CAGGS}-SP6polymerase-PolyA_{□Globin} | - | through transfection SP6 polymerase expression for testing through transfection |
| pCAG-ySUMO-GFPinsert | P_{CAGGS}-ySUMO with GFP insert for EZ-L287-PolyA_{□Globin} | - | Testing of small RNA fragment eToehold activation |
| pLenti CMV Puro DEST | P_{CMV}-CmR | P_{PGK}-PuroR | Base vector for Lentivirus cloning |
| psPAX2 | P_{CMV}-HIV-1 gag-HIV-1 pol | - | Helper vector for Lentiviral production |
| PMD2.G | P_{CMV-}VSV-G | - | Helper vector for Lentiviral production |
| pXR1 | P_{T7}-IRES_{ECMV}-mKate-PolyA_{SV40} | - | Base vector for cloning |
| pXR5NP | P_{T7}-IRES_{PSIV(Third_Pseudoknot_Deleted)}-mKate-PolyA_{SV40} | - | Negative control for IRES testing through transfection |
| pXR8 | P_{T7}-IRES_{HCVd20}-mKate-PolyA_{SV40} | - | Testing of HCV (with deleted microRNA binding site) IRES through transfection |
| pXT-T7p-sfGFP | P_{T7}-sfGFP | - | Testing of T7 promoter leakiness through transfection |
| pXT-T7CGGp-sfGFP | P_{T7CGG}-sfGFP | - | Testing of T7CGG variant promoter leakiness through transfection |
| pXT-T3p-sfGFP | P_{T3}-sfGFP | - | Testing of T3 promoter leakiness through transfection |
| pXT-SP6p-sfGFP | P_{SP6}-sfGFP | - | Testing of SP6 promoter leakiness |
| EZ-L1 | YARCdelta5 integration of P_{GAL1}-GFP-T_{ADH1} | - | Through transfection Integration of trigger RNA into yeast genome |
| EZ-L13 | P_{T7}-IRES_{KBV}-mKate-PolyA_{SV40} | - | Testing of Kashmir Bee Virus (KBV) IRES through transfection |
| EZ-L14 | P_{T7}-IRES_{ABPV}-mKate-PolyA_{SV40} | - | Testing of Acute Bee Paralysis Virus (ABPV) IRES through transfection |
| EZ-L15 | P_{T7}-IRES_{SINV-1}-mKate-PolyA_{SV40} | - | Testing of Solenopsis invicta virus-1 (SINV-1) IRES through transfection |
| EZ-L16 | P_{T7}-IRES_{ALPV}-mKate-PolyA_{SV40} | - | Testing of Aphid Lethal Paralysis Virus (ALPV) IRES through transfection |
| EZ-L17 | P_{T7}-IRES_{CrPV}-mKate-PolyA_{SV40} | - | Testing of Cricket Paralysis Virus (CrPV) IRES through transfection |
| EZ-L22 | P_{CAGGS-}GFP | - | Constitutive GFP for transfection testing |
| EZ-L39 | P_{T7}-IRES_{PSIV}-mKate-PolyA_{SV40} | - | Testing of Plauta Stali Intestine Virus (PSIV) IRES through transfection |
| EZ-L64 | P_{T7}-IRES_{CrPV}_{_}_{Site} _{Combination}₁₋₈_{_for_}_{GFP}-mKate-PolyA_{SV40} | - | Testing of CrPV site combination 1-8 through transfection |
| EZ-L65 | P_{T7}-IRES_{CrPV}_{_}_{Site} _{Combination}₁₋₂_{_for_}_{GFP}-mKate-PolyA_{SV40} | - | Testing of CrPV site combination 1-2 through transfection |
| EZ-L66 | P_{T7}-IRES_{CrPV}_{_}_{Site} _{Combination}₁₋₄_{_for_}_{GFP}-mKate-PolyA_{SV40} | - | Testing of CrPV site combination 1-4 through transfection |
| EZ-L67 | P_{T7}-IRES_{CrPV}_{_}_{Site} _{Combination}_{1-5_}_{for_}_{GFP}-mKate-PolyA_{SV40} | - | Testing of CrPV site combination 1-5 through transfection |
| EZ-L68 | P_{T7}-IRES_{CrPV_}_{Site} _{Combination} _{1-7_ for}__{GFP}-mKate-PolyA_{SV40} | - | Testing of CrPV site combination 1-7 through transfection |
| EZ-L70 | P_{T7}-IRES_{CrPV_}_{Site} _{Combination} _{8-2_ for}__{GFP}-mKate-PolyA_{SV40} | | Testing of CrPV site combination 8-2 through transfection |
| EZ-L87 | P_{CMV}-Azurite | - | Constitutive Azurite for transfection testing |
| EZ-L88 | P_{CMV}-ySUMO | - | Constitutive ySUMO for transfection testing |
| EZ-L100 | P_{T7}-IRES_{CrPV}__{Site} _{Combination} _{1-6_}_{for_}_{GFP}-mKate-PolyA_{SV40} | - | Testing of CrPV site combination 1-6 through transfection |
| EZ-L129 | P_{T7}-IRES_{CrPV}_{_}_{Site} _{Combination} _{2-6_}_{for_}_{GFP}-mKate-PolyA_{SV40} | - | Testing of CrPV site combination 1-6 through transfection |
| EZ-L139 | P_{T7}-IRES_{CrPV}_{_}_{Site} _{Combination} _{8-7_}_{for_}_{GFP}-mKate-PolyA_{SV40} | - | Testing of CrPV site combination 8-7 through transfection |
| EZ-L140 | P_{T7}-IRES_{CrPV}_{_}_{Site} _{Combination} _{8-6_}_{for_}_{GFP}-mKate-PolyA_{SV40} | - | Testing of CrPV site combination 8-6 through transfection |
| EZ-L154 | P_{T7}-IRES_{CrPV}_{_Site} _{Combination} _{6-7_}_{for_}_{GFP}-mKate-PolyA_{SV40} | - | Testing of CrPV site combination 6-7 through transfection |
| EZ-L162 | P_{T7}-IRES_{CrPV}_{_}_{Site} _{Combination} _{5-6_}_{for_}_{GFP}-mKate-PolyA_{SV40} | - | Testing of CrPV site combination 5-6 through transfection |
| EZ-L164 | P_{T7}-IRES_{CrPV_}_{Site} _{Combination} _{4-6_}_{for_}_{GFP}-mKate-PolyA_{SV40} | - | Testing of CrPV site combination 4-6 through transfection |
| EZ-L174 | P_{T7}- IRES_{CrPV_}_{Site} _{Combination} ₆₋ ₇_{_Annealingtemperature=34_}_{for}__{GFP}-mKate | - | Optimization of annealing temperature for site combination 6-7 through transfection |
| EZ-L175 | P_{T7}- IRES_{CrPV_}_{Site} _{Combination} ₆₋_{7_}_{Annealingtemperature=42_}_{for_}_{GFP}-mKate | - | Optimization of annealing temperature for site combination 6-7 through transfection |
| EZ-L176 | P_{T7}- IRES_{CrPV_}_{Site} _{Combination} ₆₋ ₇_{_Annealingtemperature=46_}_{for_}_{GFP}-mKate | - | Optimization of annealing temperature for site combination 6-7 through transfection |
| EZ-L178 | P_{T7}- IRES_{CrPV_}_{Site} _{Combination} ₈₋ _{6_}_{Annealingtemperature=30_}_{for_}_{GFP}-mKate | - | Optimization of annealing temperature for site combination 8-6 through transfection |
| EZ-L179 | P_{T7}- IRES_{CrPV_}_{Site} _{Combination} ₈₋ _{6_}_{_Annealingtemperature=34_}_{for_}_{GFP}-mKate | - | Optimization of annealing temperature for site combination 8-6 through transfection |
| EZ-L180 | P_{T7}- IRES_{CrPV_}_{Site} _{Combination} ₈₋ _{6_}_{Annealingtemperature=42_}_{for}__{GFP}-mKate | - | Optimiztion of annealing temperature for site combination 8-6 through transfection |
| EZ-L181 | P_{T7}- IRES_{CrPV_}_{Site} _{Combination} ₈₋ _{6_}_{_Annealingtemperature=46_}_{for}__{GFP}-mKate | - | Optimization of annealing temperature for site combination 8-6 through transfection |
| EZ-L183 | P_{T7}- IRES_{KBV}-IRFP | P_{TDH3}-T7polymerase- T_{ADH1} | Integration vector for the his3 locus of EZy12 to make EZy13 |
| EZ-L184 | P_{T7}-IRES_{KBV_}_{Site} _{Combination} _{8-6_ for_}_{GFP}-iRFP | P_{TDH3}-T7polymerase- T_{ADH1} | Integration vector for the his3 locus of EZy12 to make EZy14 |
| EZ-L185 | P_{T7}- IRES_{CrPV}-iRFP | P_{TDH3}-T7polymerase- T_{ADH1} | Integration vector for the his3 locus of EZy12 to make EZy15 |
| EZ-L186 | P_{T7}- IRES_{CrPV}_{_Site} _{Combination} _{8-6_}_{for_}_{GFP}-iRFP | P_{TDH3}-T7polymerase- T_{ADH1} | Integration vector for the his3 locus of EZy12 to make EZy16 |
| EZ-L187 | P_{T7}- IRES_{ABPV_}_{Site} _{Combination} _{8-6_}_{for_}_{GFP}-iRFP | P_{TDH3}-T7polymerase- T_{ADH1} | Integration vector for the his3 locus of EZy12 to make EZy17 |
| EZ-L197 | P_{T7}-IRES_{CrPV}_{_}_{Site} _{Combination}_{5-7_}_{for_}_{GFP}-mKate-PolyA_{SV40} | - | Testing of CrPV site combination 5-7 through transfection |
| EZ-L198 | P_{T7}-IRES_{CrPV}_{_}_{Site} _{Combination 2-7_}_{for_}_{GFP}-mKate-PolyA_{SV40} | - | Testing of CrPV site combination 2-7 through transfection |
| EZ_L205 | P_{T7}-IRES_{CrPV_}_{Site} _{Combination}₈₋ _{6_}_{PseudoknotBreaking_}_{for}__{GFP}-mKate-PolyA_{SV40} | - | Testing of CrPV site combination 8-6 (with Pseudoknot Breaking sequence) through transfection |
| EZ_L206 | P_{T7}-IRES_{CrPV}_{_Site} _{Combination} ₆₋ _{7_}_{PseudoknotBreaking_}_{for}__{OFP}-mKate-PolyA_{SV40} | - | Testing of CrPV site combination 6-7 (with Pseudoknot Breaking sequence) through transfection |
| EZ-L208 | P_{SP6}-UAF2-IRES_{CrPV_}_{Site} _{Combination}₆₋_{7_}_{PseudoknotBreaking_}_{for}__{GFP}-mKate-PolyA_{SV40} | - | Testing of leakiness reduction for CrPV site combination 6-7 through transfection |
| EZ-L209 | P_{SP6}-UAF2-IRES_{CrPV_Site} _{Combination} ₈₋ _{6_}_{PseudoknotBreaking_}_{for}__{GFP}-mKate-PolyA_{SV40} | - | Testing of leakiness reduction for CrPV site combination 8-6 through transfection |
| EZ-L212 | PT7-IRES_{CrPV} -sfGFP-PolyA_{SV40} | - | Transcribed for in vitro testing in extract |
| EZ-L213 | PT7-IRES_{CrPV_}_{Site} _{Combination} ₈₋₆_{_AnnealingTemperature=30_}_{PseudoknotBreaking_}_{for_yeast} _{SUMO}-sfGFP-PolyA_{SV40} | - | Transcribed for in vitro testing in extract |
| EZ-L214 | PT7-IRES_{CrPV_}_{Site} _{Combination} ₈₋ _{6_}_{AnnealingTemperature=34}_{_PseudoknotBreaking_}_{for_yeast} _{SUMO}-sfGFP-PolyA_{SV40} | - | Transcribed for in vitro testing in extract |
| EZ-L215 | PT7-IRES_{CrPV_}_{Site} _{Combination} ₈₋_{6_}_{AnnealingTemperature=38}_{_PseudoknotBreaking_}_{for_yeast} _{SUMO}-sfGFP-PolyA_{SV40} | - | Transcribed for in vitro testing in extract |
| EZ_L220 | P_{SP6}-UAF2-IRES_{CrPV_}_{Site} _{Combination} ₈₋ _{6_}_{PseudoknotBreaking_}_{for_}_{GFP_}_{AnnealMutation1}-mKate-PolyA_{SV40} | - | Testing of CrPV site combination 8-6 sensitivity through transfection |
| EZ_L221 | Ps_{P6}-UAF2-IRES_{crPv_}_{Site} _{Combination} ₈₋_{6_}_{PseudoknotBreaking_}_{for_}_{GFP_}_{AnnealMutation2}-mKate-PolyA_{SV40} | - | Testing of CrPV site combination 8-6 sensitivity through transfection |
| EZ_L222 | Ps_{P6}-UAF2-IRES_{crPv_}_{Site} _{Combination}₈₋_{6_}_{PseudoknotBreaking_}_{for_}_{GFP_}_{MutationOutside2}-mKate-PolyA_{SV40} | - | Testing of CrPV site combination 8-6 sensitivity through transfection |
| EZ_L223 | Ps_{P6}-UAF2-IRES_{crPv_}_{Site} _{Combination}₈₋_{6_}_{PseudoknotBreaking_}_{for_}_{GFP}_{_}_{MutationOutside4}-mKate-PolyA_{SV40} | - | Testing of CrPV site combination 8-6 sensitivity through transfection |
| EZ_L224 | Ps_{P6}-UAF2-IRES_{crPv_}_{Site} _{Combination}₈₋_{6_}_{PseudoknotBreaking_}_{for_}_{GFP_}_{MutationOutside6}-mKate-PolyA_{SV40} | - | Testing of CrPV site combination 8-6 sensitivity through transfection |
| EZ-L231 | P_{SP6}-UAF2⁻IRES_{KBV_}_{Site} _{Combination} ₆₋ _{7_}_{PseudoknotBreaking_}_{for_yeast} _{SUMO}-mKate-PolyA_{SV40} | - | Testing of site combination 6-7 using KBV through transfection |
| EZ-L232 | P_{SP6}-UAF2-IRES_{ABPV_}_{Site} _{Combination}₆₋ _{7_}_{PseudoknotBreaking_}_{for_yeast} _{_}_{SUMO}-mKate-PolyA_{SV40} | - | Testing of site combination 6-7 using ABPV through transfection |
| EZ-L233 | Ps_{P6}-UAF2-IRES_{KBV_}_{Site} _{Combination} ₈₋ _{6_}_{PseudoknotBreaking_}_{for_yeast}_{_}_{SUMO}-mKate-PolyA_{SV40} | | Testing of site combination 8-6 using KBV through transfection |
| EZ-L234 | P_{SP6}-UAF2-IRES_{CrPV_}_{Site} _{Combination} ₈₋_{6_}_{PseudoknotBreaking_}_{for_yeast} _{_}_{SUMO}-mKate-PolyA_{SV40} | - | Testing of orthogonality for CrPV site combination 8-6 |
| EZ-L236 | P_{SP6}-UAF2-IRES_{CrPV_}_{Site} _{Combination} ₈₋ _{6_}_{PseudoknotBreaking_}_{for}__{Azurite}-mKate-PolyA_{SV40} | - | Testing of orthogonality for CrPV site combination 8-6 |
| EZ-L287 | P_{T7}-UAF2-IRES_{ABPV_}_{Site} _{Combination} ₈₋ _{6_}_{PseudoknotBreaking_}_{for}__{GFP}-mKate-PolyA_{SV40} | - | Testing of eToehold sensitivity to short RNA similarities |
| EZ-L290 | P_{SP6}-UAF2-IRES_{ABPV_}_{Site} _{Combination} ₈₋ _{6_}_{PseudoknotBreaking_}_{for_yeast}_{_}_{SUMO}-mKate-PolyA_{SV40} | - | Testing of site combination 8-6 using ABPV through transfection |
| EZ-L330 | P_{CMV}-GFP-mKate | - | Testing of PolII driven eToeholds |
| EZ-L331 | P_{CMV}-GFP-Stopcodons-mKate | - | Testing of PolII driven eToeholds |
| EZ-L335 | P_{CMV}-GFP-Stopcodons-Stemloops-mKate | - | Testing of PolII driven eToeholds |
| EZ-L337 | P_{SP6}-UAF2-IRES_{KBV_}_{Site} _{Combination}₈₋ _{6_}_{PseudoknotBreaking_}_{for_yeast} _{_}_{SUMO}-Toehold_{KOZAK}_{_}_{sensing_yeast}_{_}_{SUMO}-mKate-PolyA_{SV40} | - | Testing of KOZAK toeholds |
| EZ-L345 | P_{CMV}-GFP-Stopcodons-Stemloops-IRES_{KBV}-mKate | - | Testing of PolII driven eToeholds |
| EZ-L348 | P_{CMV}-GFP-Stopcodons-Stemloops-IRES_{KBV}_{_Site} _{Combination} ₈₋ _{6_}_{PseudoknotBreaking_}_{for_yeast}_{_SUMO} -mKate | - | Testing of PolII driven eToeholds |
| EZ-L366 | P_{T7}-yeastSUMO | - | Expression of yeast SUMO RNA as a trigger |
| EZ-L392 | P_{T7}-GFP | - | Expression of GFP RNA as a trigger |
| EZ-L400 | P_{T7}-Stopcodons-Stemloops-IRES_{ABPV_}_{Site} _{Combination} _{8-6_}_{PseudoknotBreaking_}_{for_yeast}_{_}_{SUMO-} mKate-PolyA_{SV40} | - | Testing of Stemloops |
| EZ-L521 | P_{SFFV}-sfGFP- Stopcodons-Stemloops-IRES_{ABPV}-nanoluciferase-WPRE | - | Lentiviral vector control |
| EZ-L534 | P_{CAGGS}-T7polymerase-sfGFP-WPRE | PT7- Stopcodons-Stemloops-IRES_{CrPV_}_{Site} _{Combination} ₈₋ _{6_}_{PseudoknotBreaking}_{_for_}_{Zika}- mKate-PolyA_{SV40} | Lentiviral vector for T7 driven Zika sensor |
| EZ-L536 | P_{SFFV}-sfGFP- Stopcodons-Stemloops-IRES_{CrPV_}_{Site} _{Combination} _{8-6_}_{PseudoknotBreaking_}_{for_}_{Zika}-nanoluciferase-WPRE | - | Lentiviral vector for SFFV driven Zika sensor |
| EZ-L541 | P_{SFFV}-sfGFP- Stopcodons-Stemloops-IRES_{CrPV_}_{Site} _{Combination_}₈₋ _{6_}_{PseudoknotBreaking_}_{for_}_{Sars}_{_Cov}_{_2_}_{Spike}-nanoluciferase-WPRE | - | Lentiviral vector for SFFV driven Zika sensor |
| EZ-L542 | P_{SFFV}-sfGFP- Stopcodons-Stemloops-IRES_{ABPV_}_{Site} _{Combination} ₈₋ _{6_}_{PseudoknotBreaking_}_{for}__{Sars}_{_Cov}_{_}₂_{_3prime}-nanoluciferase-WPRE | - | Lentiviral vector for SFFV driven Zika sensor |
| EZ-L548 | P_{CMV}-GFP-Stopcodons-Stemloops-IRES_{ABPV_}_{Site} _{Combination} ₈₋ _{6_}_{PseudoknotBreaking_}_{for_}ₕₛₚ₇₀-Azurite | - | Heat shock protein sensing eToehold |
| EZ-L554 | P_{CMV}-GFP-Stopcodons-Stemloops-IRES_{CrPV_}_{Site} _{Combination} ₈₋ _{6_}_{PseudoknotBreaking_}_{for_}ₕₛₚ₄₀-Azurite | - | Heat shock protein sensing eToehold |
| EZ-L575 | P_{CMV}-GFP-Stopcodons-Stemloops-IRES_{ABPV_}_{Site} _{Combination} ₈₋ _{6_}_{PseudoknotBreaking_}_{for}__{MouseTyr}-Azurite | - | Mouse Tyrosinase sensing eToehold |
| EZ-L577 | P_{CMV}-GFP-Stopcodons-Stemloops-IRES_{CrPV_}_{Site} _{Combination} ₈₋ _{6_}_{PseudoknotBreaking_}_{for}__{MouseTyr}-Azurite | - | Mouse Tyrosinase sensing eToehold |
| EZ-L661 | P_{CMV}-iRFP | - | Testing of human specific toeholds |
| EZ-L781 | P_{CMV}-Mouse Metalloprotease 9 | - | Testing of human specific toeholds |
| EZ-L781 | P_{CMV}-Mouse Tyrosinase | - | Testing of human specific toeholds |
| EZ-L789 | P_{SFFV}-sfGFP- Stopcodons-Stemloops-IRES_{CrPV}-nanoluciferase-WPRE | - | Testing of human specific toeholds |
| EZ-L824 | P_{SFFV}-sfGFP- Stopcodons-Stemloops-IRES_{HCV_}_{Site} _{Combination} ₂₋₁₂ _{for} _{iRFP}-nanoluciferase-WPRE | - | Testing of human specific toeholds |
| EZ-L842 | P_{SFFV}-sfGFP- Stopcodons-Stemloops-IRES_{EV71mutant} -nanoluciferase-WPRE | - | Testing of human specific toeholds |
| EZ-L975 | P_{SFFV}-sfGFP- Stopcodons-Stemloops-IRES_{PolioVirus} -nanoluciferase-WPRE | - | Testing of human specific toeholds |
| EZ-L980 | P_{SFFV}-sfGFP- Stopcodons-Stemloops-IRES_{PolioVirus}_{_}_{Site} _{Combination} ₀₋₅ _{for} _{Mouse} _{Tyrosinase} - nanoluciferase-WPRE | - | Testing of human specific toeholds |
| EZ-L1027 | P_{SFFV}-sfGFP- Stopcodons-Stemloops-IRES_{PolioVirus}__{Site} _{Combination} ₁₋₂ _{for} _{MMP9} - nanoluciferase-WPRE | - | Testing of human specific toeholds |
| EZ-L1060 | P_{SFFV}-nanoluciferase-WPRE | - | Testing of human specific toeholds |
| EZ-L1064 | P_{SFFV}-sfGFP- Stopcodons-Stemloops-IRES_{EV71mutant}_{_}_{Site} _{Combination} ₀₋₁ _{for MMP9} - nanoluciferase-WPRE | - | Testing of human specific toeholds |

TABLE 2

Relative fold change in IRES RNA level in cells containing a 45-bp sequence complementary to the IRES transcript, compared to an orthogonal sequence

| | Fold change | Significance |
|---|---|---|
| HEK293T | 0.59 | N.S. |
| Human dermal fibroblasts | 0.55 | p < 0.01 |
| Human skeletal muscle | 0.67 | N.S. |

In SEQ ID NO: 12, residues 153-160 and 218-259 are GFP RNA insertions. In SEQ ID NO: 13, residues 152-186 and 197-207 are GFP RNA insertions. In SEQ ID NO: 14, residues 153-162 and 220-259 are Azurite RNA insertions. In SEQ ID NO: 15, residues 153-160 and 218-262 are ySUMO RNA insertions. In SEQ ID NO: 16, residues 159-171 and 257-302 are GFP RNA insertions. In SEQ ID NO: 17, residues 15-168 and 254-297 are ySUMO RNA insertions. In SEQ ID NO: 18, residues 153-158 and 216-254 are Zika RNA insertions. In SEQ ID NO: 19, residues 153-162 and 220-266 are SARS-COV-2 Spike RNA insertions. In SEQ ID NO: 20, residues 159-168 and 254-301 are SARS-COV-2 Spike RNA insertions. In SEQ ID NO: 21, residues 153-164 and 250-289 are hsp70 RNA insertions. In SEQ ID NO: 22, residues 153-161 and 219-259 are hsp40 RNA insertions. In SEQ ID NO: 23, residues 159-166 and 252-292 are mouse tyrosinase RNA insertions. In SEQ ID NO: 24, residues 153-159 and 217-256 are mouse tyrosinase RNA insertions. In SEQ ID NO: 25, residues 83-117 and 317-328 are iRFP RNA insertions. In SEQ ID NO: 26, residues 117-151 and 204-218 are iRFP RNA insertions. In SEQ ID NO: 27, residues 78-108 and 142-156 are mouse metalloprotease 9 RNA insertions. In SEQ ID NO: 28, residues 1-40 and 674

In SEQ ID NOs: 30-36, exemplary modified IRES sequences are presented, where “X” residues indicate the sites for insertion of first and second nucleotide sequences.

1. A recombinant nucleic acid molecule, comprising:
a) a first segment encoding a recombinant Group 1 Dicistroviridae internal ribosome entry site (IRES) that has been modified to incorporate exogenous nucleotide sequences at a first site and a second site, and
b) a second segment encoding a protein, downstream from and operably linked to the first segment such that translation of the protein is repressed when the IRES is in an inactivated state;
wherein the first site comprises a first nucleotide sequence, and the second site comprises a second nucleotide sequence which is the reverse complement of at least a portion of the first nucleotide sequence.

2. A recombinant nucleic acid molecule, comprising from 5′ to 3′:
a) a first segment encoding a recombinant viral internal ribosome entry site (IRES) that has been modified at a first site to incorporate a first exogenous nucleotide sequence and modified at a second site to incorporate a second exogenous nucleotide sequence; and
b) a second segment encoding a protein, downstream from and operably linked to the first segment such that translation of the protein is repressed when the IRES is in an inactivated state;
wherein the second nucleotide sequence is the reverse complement of at least a portion of the first nucleotide sequence.

3. The recombinant nucleic acid molecule of claim 2, wherein the IRES that is modified is a Group 1 Discistroviridae IRES; a Hepacivirus IRES; or an Enterovirus IRES.

4. The recombinant nucleic acid molecule of claim 1, wherein the IRES that is modified is an IRES from a mammalian pathogenic virus or mammalian commensal virus.

5. The recombinant nucleic acid molecule of claim 4, wherein the IRES that is modified is an IRES from a human pathogenic virus or human commensal virus.

6. The recombinant nucleic acid molecule of claim 1, wherein the nucleic acid molecule is an mRNA.

7. The recombinant nucleic acid molecule of claim 1, wherein the second nucleotide sequence is the reverse complement of substantially all of the first nucleotide sequence.

8. The recombinant nucleic acid molecule of claim 1, wherein the Group 1 Dicistroviridae IRES is a cricket paralysis virus (CrPV), a Kashmir bee virus (KBV), an acute bee paralysis virus (ABPV), a Plauta Stali Intestine Virus (PSIV) IRES; an aphid lethal paralysis virus (ALPV) IRES; a black queen cell virus (BQCV) IRES; a Drosophila C virus (DCV) IRES; a Himetobi P virus (HiPV) IRES; a Homalodisca coagulata virus-1 (HoCV-1) IRES; a Rhopalosiphum padi virus (RhPV) IRES; and a Triatoma virus (TrV) IRES .

9. The recombinant nucleic acid molecule of claim 3, wherein the Hepacivirus IRES is a hepatitis c virus (HCV) IRES or the Enterovirus IRES is a poliovirus (PV) IRES or enterovirus 71 (EV71) IRES.

10. (canceled)

11. The recombinant nucleic acid molecule of claim 1, wherein the first and second sites are each independently selected from any of Site 1, Site 2, Site 3, Site 4, Site 5, Site 6, Site 7, and Site 8.

12-19. (canceled)

20. The recombinant nucleic acid molecule of claim 1, wherein the first nucleotide sequence is the reverse complement of a sequence found in a target eukaryotic organism, target prokaryotic organism, or target virus.

21. (canceled)

22. The recombinant nucleic acid molecule of claim 20, wherein the target virus is Zika virus or a coronavirus.

23-24. (canceled)

25. The recombinant nucleic acid molecule of claim 1, wherein the first and second nucleotide sequences are capable of hybridizing when expressed in a eukaryotic cell under in vivo or in vitro conditions, causing the IRES to fold into an inactivated state, wherein the eukaryotic cell is not a plant cell.

26. The recombinant nucleic acid molecule of claim 1, wherein the IRES is configured to fold into an activated state in the presence of a trigger RNA molecule comprising a third nucleotide sequence, wherein the third nucleotide sequence is the reverse compliment of the first nucleotide sequence of the recombinant nucleic acid molecule of claim 1.

27. The recombinant nucleic acid molecule of claim 26, wherein the first nucleotide sequence is capable of hybridizing to the third nucleotide sequence when expressed in a eukaryotic cell under in vivo conditions, causing the IRES to fold into the activated state, wherein the eukaryotic cell is not a plant cell.

28. An expression construct comprising a sequence encoding or comprising the recombinant nucleic acid molecule of claim 1 , and further comprising, 5′ and/or 3′ of the sequence encoding or comprising the recombinant nucleic acid molecule of any of claims 1-27, one or more of:
a) an IRES pseudoknot sequence;
b) an IRES pseudoknot sequence found in the wild-type sequence of a virus in which the IRES naturally occurs;
c) a promoter and/or upstream activating factor binding sequence;
d) a stop codon;
e) a stem-loop;
f) a 5′ cap;
g) a reporter gene; and
h) a poly-A tail.

29-31. (canceled)

32. A recombinant mRNA molecule, comprising:
a) a first segment encoding a first protein,
b) a second segment, downstream of the first segment, encoding a recombinant Group 1 Dicistroviridae internal ribosome entry site (IRES) that has been modified to incorporate exogenous nucleotide sequences at a first site and a second site, and
c) a third segment encoding a second protein, downstream from and operably linked to the second segment such that translation of the second protein is repressed when the IRES is in an inactivated state;
wherein transcription of the recombinant mRNA molecule is dependent on a polymerase, and wherein the first site comprises a first nucleotide sequence, and the second site comprises a second nucleotide sequence which is the reverse complement of at least a portion of the first nucleotide sequence.

33-36. (canceled)

37. A system for the control of gene expression, comprising:
a) the recombinant nucleic acid molecule of claim 1 ; and
b) a trigger RNA molecule comprising a third nucleotide sequence, wherein the third nucleotide sequence is the reverse compliment of the first nucleotide sequence of the recombinant nucleic acid molecule.

38. (canceled)

39. A method of activating and/or modulating expression of a protein, comprising:
a) providing a eukaryotic cell engineered to express the recombinant nucleic acid molecule of claim 1; and
b) introducing a trigger RNA molecule comprising a third nucleotide sequence into the eukaryotic cell, wherein the third nucleotide sequence is the reverse compliment of the first nucleotide sequence of the recombinant nucleic acid molecule;
wherein the first nucleotide sequence hybridizes to the third nucleotide sequence under in vivo conditions, causing the IRES to fold into an activated state, further wherein the eukaryotic cell is not a plant cell.

40. (canceled)

41. A method for detecting viral infection of a eukaryotic cell, comprising:
a) providing a eukaryotic cell engineered to express the recombinant nucleic acid molecule of claim 1, wherein the first nucleotide sequence of the recombinant nucleic acid molecule is configured to be the reverse compliment of at least a portion of a mRNA sequence unique to a virus; and
b) determining whether the eukaryotic cell is infected with the virus by detecting and/or measuring the presence of the protein encoded by the second segment of the recombinant nucleic acid molecule, wherein the eukaryotic cell is not a plant cell.

42-45. (canceled)
